(19) United States
(12) Patent Application Publication
(10) Pub. No.: US 2021/0134398 A1
(43) Pub. Date: May 6, 2021

(54) Combinatorial Chemistry Computational System and Enhanced Selection Method

(71) Applicant: Southern Methodist University, Dallas, TX (US)

(72) Inventors: John G. Wise, Dallas, TX (US); Pia D. Vogel, Dallas, TX (US)

(21) Appl. No.: 17/091,436

(22) Filed: Nov. 6, 2020

Related U.S. Application Data
(60) Provisional application No. 62/931,333, filed on Nov. 6, 2019.

(51) Int. Cl. G16C 20/60; G16C 20/10

(52) U.S. Cl. CPC G16C 20/60; G16C 20/10

(57) ABSTRACT

A method for identifying a potentially useful molecular combination includes applying a selection procedure to a compound to identify a first set of candidate molecules, the procedure including providing a chemical synthesis scheme, a virtual scaffold molecule of the compound, and a virtual reactant fragment to react with the scaffold molecule according to the scheme; preparing the reactant fragment and the scaffold molecule for analyzing combinations of them; designating a remaining scaffold subset and a remaining fragment subset if a product molecule can be formed from them; rotating the fragment subset about an axis connecting the scaffold subset and the fragment subset incrementally through 360 degrees; and identifying potentially useful combinations of the reactant fragment and the scaffold molecule; identifying a set of combinatorial fragments from the first set of candidates; and applying the selection procedure to the set of combinatorial fragments to identify a second set of candidate molecules.

### CROSS-REFERENCE TO RELATED APPLICATIONS

This application claims priority to U.S. Provisional Application Ser. No. 62/931,333, filed Nov. 6, 2019, the entire contents of which are incorporated herein by reference.

### STATEMENT OF FEDERALLY FUNDED RESEARCH

This invention was made with government support under GM094771 awarded by the National Institute of Health. The government has certain rights in the invention.

### TECHNICAL FIELD OF THE INVENTION

The present invention relates in general to the field of drug development. In particular, the present invention relates to modeling molecular syntheses in silico to identify likely promising combinations, and to eliminate likely unsuccessful combinations before synthesizing and experimenting with combinations.

### BACKGROUND OF THE INVENTION

Without limiting the scope of the invention, its background is described in connection with drug development. In particular the invention is described in connection with a use of one or more computer programs to model molecular syntheses.

In the last few decades, the quality and efficiency of scientific and technological tools that are important for progress in biopharmaceutical discovery and research have improved significantly. For example, DNA sequencing has become over a billion times faster since the first genome sequences were determined in the 1970s. The improvements in drug discovery tools should enable identification and evaluation of therapeutic candidates that have high reliability, efficacy, reproducibility, and safety. However, therapeutic candidates today are more likely to fail in clinical trials than those identified in the 1970s, while the cost to develop and gain marketing approval for a new therapeutic agent has increased to approximately $2.6 billion.

Organic chemistry is a rate-limiting factor in drug discovery. Investments in organic chemistry from drug discovery applications have decreased over time, with more focus toward applied research such as translational medicine and biomarker development. There are a number of chemical synthesis challenges in that most drug candidates contain amines, N-heterocycles, and unprotected polar groups that add to the complexity in synthesis.

In drug development program, especially in lead development and optimization, large sets of molecules that are related to a known molecule of interest are often synthesized with the intent of identifying derivative molecules that have better drug-like characteristics than those observed in the assays of the originally identified molecule. These organic chemistry syntheses are expensive and time-consuming, and they can be exceedingly laborious, requiring the skills and time of a team of highly qualified chemists. While some drug-like characteristics can be estimated using computational approaches, others require assessment by biochemical, biophysical, pharmacological, cell-biological, or animal experimentation. Often, hundreds or thousands of compounds are synthesized, with only a few variants showing desired improvement in pharmacological characteristics.

The prior art features Cramer et al. 1998 (likely the earliest available virtual chemistry program description); Krier et al. 2005 (uses a compound scaffold with linkers containing the functional group for reaction); Srinivasan et al. 2006 (uses pre-specified “click” chemistry to which the present invention is not restricted); Melnikov et al. 2007 (focuses on quantitative structure-activity relationships of the compounds); and Durrant and McCammon 2012 (uses pre-specified “click” chemistry to which the present invention is not restricted).

Another prior art method for combinatorial in silico drug-lead optimization exists, which the present invention uses. This prior art method is described below, and the present invention is distinguished from it.

It is desirable to improve on existing methods to enhance and accelerate organic synthesis chemistry through the use of computational systems and methods to identify likely useful molecular combinations.

### SUMMARY OF THE INVENTION

In some embodiments of the disclosure, a system for identifying one or more potentially useful molecular combinations is disclosed as including applying a selection procedure to a compound of interest to identify a first set of one or more candidate molecules, the selection procedure including: providing a chemical synthesis scheme for a compound of interest, a virtual scaffold molecule of the compound of interest, and a virtual reactant fragment to react with the virtual scaffold molecule according to the chemical synthesis scheme; preparing the virtual reactant fragment and the virtual scaffold molecule for analyzing combinations of the virtual reactant fragment and the virtual scaffold molecule; designating a remaining scaffold subset and a remaining fragment subset if a product molecule can be formed from the virtual scaffold molecule and the virtual reactant fragment; rotating the remaining fragment subset about an axis connecting the remaining scaffold subset and the remaining fragment subset through 360 degrees in increments of less than or equal to 5 degrees; and identifying potentially useful combinations of the virtual reactant fragment and the virtual scaffold molecule, by: recording as a potential product increment each increment at which a steric collision is not detected; and recording a separation distance between the remaining fragment subset and the remaining scaffold subset at each increment and identifying a set of product increments for which the separation distances are less than or equal to a predetermined criterion distance to identify the one or more potentially useful molecular combinations; identifying a set of combinatorial fragments from the first set of one or more candidates; and applying the selection procedure to the set of combinatorial fragments to identify a second set of one or more candidate molecules that are the one or more potentially useful molecular combinations. In one aspect, the preparing the virtual reactant fragment and the virtual scaffold molecule includes providing a three-dimensional coordinate system for the virtual reactive fragment. In another aspect, the preparing the virtual reactant fragment and the virtual scaffold molecule includes identifying a fragment alignment atom and a fragment root atom in the virtual reactant fragment; and the preparing the virtual reactant fragment and the virtual scaffold molecule includes: identifying a scaffold alignment atom and a scaffold root atom in the virtual scaffold molecule; and providing a three-dimensional coordinate system for the virtual scaffold molecule and aligning the scaffold root atom with an origin and the scaffold alignment atom with an x-axis. In another aspect, the preparing the virtual reactant fragment and the virtual scaffold molecule includes: aligning the fragment alignment atom with the scaffold root atom; and aligning the fragment root atom with the scaffold alignment atom. In another aspect, the axis connecting the remaining scaffold subset and the remaining fragment subset is defined by the scaffold root atom and the virtual root atom. In another aspect, the identifying potentially useful combinations further includes creating a product file for a configuration of the remaining fragment subset and the remaining scaffold subset at each increment of the set of product increments.

In some embodiments of the disclosure, a non-transitory computer-readable medium encoded with a computer program for execution by a processor for identifying one or more potentially useful molecular combinations is disclosed, with the computer program including instructions for applying a selection procedure to a compound of interest to identify a first set of one or more candidate molecules, the selection procedure including: receiving a chemical synthesis scheme for a compound of interest, a virtual scaffold molecule of the compound of interest, and a virtual reactant fragment to react with the virtual scaffold molecule according to the chemical synthesis scheme; receiving input to prepare the virtual reactant fragment and the virtual scaffold molecule for analyzing combinations of the virtual reactant fragment and the virtual scaffold molecule; designating a remaining scaffold subset and a remaining fragment subset if a product molecule can be formed from the virtual scaffold molecule and the virtual reactant fragment; rotating the remaining fragment subset about an axis connecting the remaining scaffold subset and the remaining fragment subset through 360 degrees in increments of less than or equal to 5 degrees; identifying potentially useful combinations of the virtual reactant fragment and the virtual scaffold molecule by: recording as a potential product increment each increment at which a steric collision is not detected; and recording a separation distance between the remaining fragment subset and the remaining scaffold subset at each increment and identifying a set of product increments for which the separation distances are less than or equal to a predetermined criterion distance, to identify the first set of one or more candidate molecules; identifying a set of combinatorial fragments from the first set of one or more candidates; and applying the selection procedure to the set of combinatorial fragments to identify a second set of one or more candidate molecules that are the one or more potentially useful molecular combinations. In one aspect, the preparing the virtual reactant fragment and the virtual scaffold molecule includes providing a three-dimensional coordinate system for the virtual reactive fragment. In another aspect, the preparing the virtual reactant fragment and the virtual scaffold molecule includes identifying a fragment alignment atom and a fragment root atom in the virtual reactant fragment; and the preparing the virtual reactant fragment and the virtual scaffold molecule includes: identifying a scaffold alignment atom and a scaffold root atom in the virtual scaffold molecule; and providing a three-dimensional coordinate system for the virtual scaffold molecule and aligning the scaffold root atom with an origin and the scaffold alignment atom with an x-axis. In another aspect, the preparing the virtual reactant fragment and the virtual scaffold molecule includes: aligning the fragment alignment atom with the scaffold root atom; and aligning the fragment root atom with the scaffold alignment atom. In another aspect, the axis connecting the remaining scaffold subset and the remaining fragment subset is defined by the scaffold root atom and the virtual root atom. In another aspect, the identifying potentially useful combinations further includes creating a product file for a configuration of the remaining fragment subset and the remaining scaffold subset at each increment of the set of product increments.

In some embodiments of the disclosure, an apparatus is disclosed as including a processor; a memory communicably coupled to the processor; an output device communicably coupled to the processor; and a non-transitory computer-readable medium encoded with a computer program for execution by the processor that causes the processor to: apply a selection procedure to a compound of interest to identify a first set of one or more candidate molecules, the selection procedure including: receiving a chemical synthesis scheme for a compound of interest, a virtual scaffold molecule of the compound of interest, and a virtual reactant fragment to react with the virtual scaffold molecule according to the chemical synthesis scheme; receiving input to prepare the virtual reactant fragment and the virtual scaffold molecule for analyzing combinations of the virtual reactant fragment and the virtual scaffold molecule; designating a remaining scaffold subset and a remaining fragment subset if a product molecule can be formed from the virtual scaffold molecule and the virtual reactant fragment; rotating the remaining fragment subset about an axis connecting the remaining scaffold subset and the remaining fragment subset through 360 degrees in increments of less than or equal to 5 degrees; and identifying potentially useful combinations of the virtual reactant fragment and the virtual scaffold molecule, by: recording as a potential product increment each increment at which a steric collision is not detected; and recording a separation distance between the remaining fragment subset and the remaining scaffold subset at each increment and identifying a set of product increments for which the separation distances are less than or equal to a predetermined criterion distance, to identify the first set of one or more candidate molecules; identify a set of combinatorial fragments from the first set of one or more candidates; and apply the selection procedure to the set of combinatorial fragments to identify a second set of one or more candidate molecules that are the one or more potentially useful molecular combinations. In one aspect, the preparing the virtual reactant fragment and the virtual scaffold molecule includes providing a three-dimensional coordinate system for the virtual reactive fragment. In another aspect, the preparing the virtual reactant fragment and the virtual scaffold molecule includes identifying a fragment alignment atom and a fragment root atom in the virtual reactant fragment; and the preparing the virtual reactant fragment and the virtual scaffold molecule includes: identifying a scaffold alignment atom and a scaffold root atom in the virtual scaffold molecule; and providing a three-dimensional coordinate system for the virtual scaffold molecule and aligning the scaffold root atom with an origin and the scaffold alignment atom with an x-axis. In another aspect, the preparing the virtual reactant fragment and the virtual scaffold molecule includes: aligning the fragment alignment atom with the scaffold root atom; and aligning the fragment root atom with the scaffold alignment atom. In another aspect, the axis connecting the remaining scaffold subset and the remaining fragment subset is defined by the scaffold root atom and the virtual root atom. In another aspect, the identifying potentially useful combinations further includes creating a product file for a configuration of the remaining fragment subset and the remaining scaffold subset at each increment of the set of product increments.

### BRIEF DESCRIPTION OF THE DRAWINGS

For a more complete understanding of the features and advantages of the present invention, reference is now made to the detailed description of the invention along with the accompanying figures, in which:

FIG. 1A shows a flowchart for a prior art method for identifying one or more potentially pharmacologically useful molecular combinations.

FIG. 1B shows another flowchart for the prior art method for identifying one or more potentially pharmacologically useful molecular combinations.

FIG. 2 shows a flowchart for a method embodiment of the present invention.

FIGS. 3A and 3B depict chemical synthesis schemes for an exemplary compound of interest, SMU 29.

FIGS. 4A and 4B depict chemical synthesis schemes for an exemplary compound of interest, SMU 45.

FIGS. 5A, 5B, and 5C show aspects of preparing a virtual reactant fragment and a virtual scaffold molecule for analyzing combinations.

FIG. 6A shows compound SMU-29, and FIGS. 6B, 6C, and 6D show views of SMU-29 and P-glycoprotein (“P-gp”) docked.

FIGS. 7A and 7B show a central retrosynthetic disconnection of a carbon-sulfur bond in SMU-29 and a generalization for virtual synthesis, respectively.

FIG. 8 shows 647 virtual SMU-29-variants aligned on the heavy atoms of the common 1-phenyl-3-(2,4,5-trimethylphenyl)-1H-pyrazol-5-yl group.

FIG. 9A shows SMU-29 and FIG. 9B shows an overlay of the 12 variants shown in Table 1 in the putative allosteric site on P-gp.

FIG. 10 shows a synthesis scheme for five compounds selected for chemical synthesis and subsequent testing for potentially improved efficacy in cancer cell culture.

FIG. 11A shows the structures of Group 1 variants 29-216 (216), 29-227 (227), 29-231 (231), 29-541 (541) and 29-551 (551) in the highest estimated affinity docking pose in the putative allosteric site of P-gp. FIG. 11B shows the chemical structures of the 29 variants underneath the respective docking images.

FIG. 12 shows the cell viability of SMU-29 and the Group 1 variants on sensitizing the chemotherapy-resistant prostate cancer cell line, DU145TXR, to paclitaxel, at concentrations of 3 μM, 5 μM, 7 μM, and 10 μM.

FIG. 13A shows the relative fluorescence of cellular calcein measured over time and FIG. 13B shows similar calcein accumulation assays performed after a 6-hour pre-incubation with the SMU-29-variants and parental compound SMU-29.

FIG. 14A shows five synthesized structural derivatives of 29 (“Group 2 variants”) that varied in size, shape, polar surface area, as well as overall hydrophobicity as judged by calculated values of molecular weight, topological polar surface area and log P, and FIG. 14B shows the chemical structures of the variants underneath the respective docking images.

FIG. 15 shows the cell viability of the Group 2 structural 29-variants on sensitizing the chemotherapy-resistant prostate cancer cell line, DU145TXR, to paclitaxel, at concentrations of 3 M, 5 μM, 7 μM, and 10 μM.

FIG. 16A shows considerably increased calcein accumulation in DU145TXR cells when compared to 29 in the presence of 5 μM of compounds 238 and 255, without pre-incubation, and FIG. 16B shows an increased calcein accumulation with pre-incubation for variant 29-278.

FIG. 17 shows the results of assays that measured the intracellular accumulation of the experimental compounds using LC-MS/MS methods after incubation with the P-gp over-expressing cell line, DU145TXR, in the absence and presence of the strong P-gp inhibitor, tariquidar⁴⁷(TQR).

FIG. 18 shows a pronounced steric clash of P-gp amino acid side chains with the bound inhibitor when P-gp adopts a conformation similar to that of the cryo-EM structure.

FIG. 19 shows Diazoacetonitrile.

FIG. 20 shows 3-oxo-3-(2,4,5-trimethylphenyl)propanenitrile.

FIG. 21 shows 1-phenyl-3-(2,4,5-trimethylphenyl)-1H-pyrazol-5-amine.

FIG. 22 shows 2-chloro-N-[1-phenyl-3-(2,4,5-trimethylphenyl)-1H-pyrazol-5-yl]acetamide.

FIG. 23 shows 2-(acetylsulfanyl)-N-[1-phenyl-3-(2,4,5-trimethylphenyl)-1H-pyrazol-5-yl]acetamide.

FIG. 24 shows 2-mercapto-N-(1-phenyl-3-(2,4,5-trimethylphenyl)-1H-pyrazol-5-yl)acetamide.

FIG. 25 shows 2-chloro-N-(naphthalen-2-yl) acetamide.

FIG. 26 shows 2-chloro-1-(10,11-dihydro-5H-dibenzo[b]azepin-5-yl)ethan-1-one.

FIG. 27 shows 2-chloro-N-(3,4,5-trimethoxyphenyl)acetamide.

FIG. 28 shows N-(benzo[d]thiazol-6-yl)-2-chloroacetamide.

FIG. 29 shows N-((3s,5s,7s)-adamantan-1-yl)-2-chloroacetamide.

FIG. 30 shows N-benzhydryl-2-chloroacetamide.

FIG. 31 shows 2-chloro-N-(4-fluorobenzyl)acetamide.

FIG. 32 shows 2-chloro-1-(9H-fluoren-2-yl)ethan-1-one.

FIG. 33 shows 2-{[2-(9H-flouoren-2-yl)-2-oxoethyl]}-N-[1-phenyl-3-(2,4,5-trimethylphenyl0-1H-pyrazol-5-yl]acetamide.

FIG. 34 shows 2-[(2-{2-azatricyclo[9.4.0.0]pentadeca-1(11),3(8),4,6,12,14-hexaen-2-yl}-2-oxoethyl)sulfanyl]-N-[1-phenyl-3-(2,4,5-trimethylphenyl)-1H-pyrazol-5-yl]acetamide.

FIG. 35 shows 2-({[(naphthalen-2-yl)carbamoyl]methyl}sulfanyl)-N-[1-phenyl-3-(2,4,5-trimethylphenyl)-1H-pyrazol-5-yl]acetamide.

FIG. 36 shows N-[1-phenyl-3-(2,4,5-trimethylphenyl)-1H-pyrazol-5-yl]-2-({[(3,4,5-trimethoxyphenyl)carbamoyl]methyl}sulfanyl)acetamide.

FIG. 37 shows 2-({[(1,3-benzothiazol-6-yl)carbamoyl]methyl}sulfanyl)-N-[1-phenyl-3-(2,4,5 trimethylphenyl)-1H-pyrazol-5-yl]acetamide.

FIG. 38 shows 2-({[(adamantan-1-yl)carbamoyl]methyl}sulfanyl)-N-[1-phenyl-3-(2,4,5-trimethylphenyl)-1H-pyrazol-5-yl]acetamide.

FIG. 39 shows N-(diphenylmethyl)-2-[({[1-phenyl-3-(2,4,5-trimethylphenyl)-1H-pyrazol-5-yl]carbamoyl}methyl)sulfanyl]acetamide.

FIG. 40 shows N-[(4-fluorophenyl)methyl]-2-[{[1-phenyl-3-(2,4,5-trimethylphenyl)-1H-pyrazol-5-yl]carbamoyl}methyl)sulfanyl]acetamide.

FIG. 41 shows 5H-[1,2,4]triazino[5,6-b]indole-3-thiol.

FIG. 42 shows 2-((5H-[1,2,4]triazino[5,6-b]indol-3-yl)thio)-N-(1-phenyl-3-(2,4,5-trimethylphenyl)-1H-pyrazol-5-yl)acetamide.

FIG. 43 shows 5-bromonicotinoyl chloride.

FIG. 44 shows 5-bromo-N-(3-mercaptophenyl)nicotinamide.

FIG. 45 shows 5-bromo-N-(3-((2-oxo-2-((1-phenyl-3-(2,4,5-trimethylphenyl)-1H-pyrazol-5-yl)amino)ethyl)thio)phenyl)nicotinamide.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

An objective of the present invention is an increase in the efficiency of drug development programs by performing iterative molecular syntheses using efficient computational approaches, instead of synthesizing large sets of compounds related to a molecule of interest using organic chemistry synthesis methods. The present invention allows the synthesis of large numbers of variants in silico to inform choices of which of the vast numbers of possible compounds to synthesize for experimentation, saving expense and time in the drug development process. After in silico production, the virtual compound variants are computationally assessed for any predicted improvements in pharmacological characteristics, including any characteristic that can be calculated, for example, physicochemical data, such as total polar surface area, log P values, molecular weight, etc. and more complex indicators of improved drug-like characteristics, for example, predicted toxicities, mutagenicity, likelihood of inducing potential drug-drug interactions (cytochrome P450 isozyme substrate character, etc.), increased binding affinities to targeted proteins, decreased bonding affinities to undesired protein targets, and more.

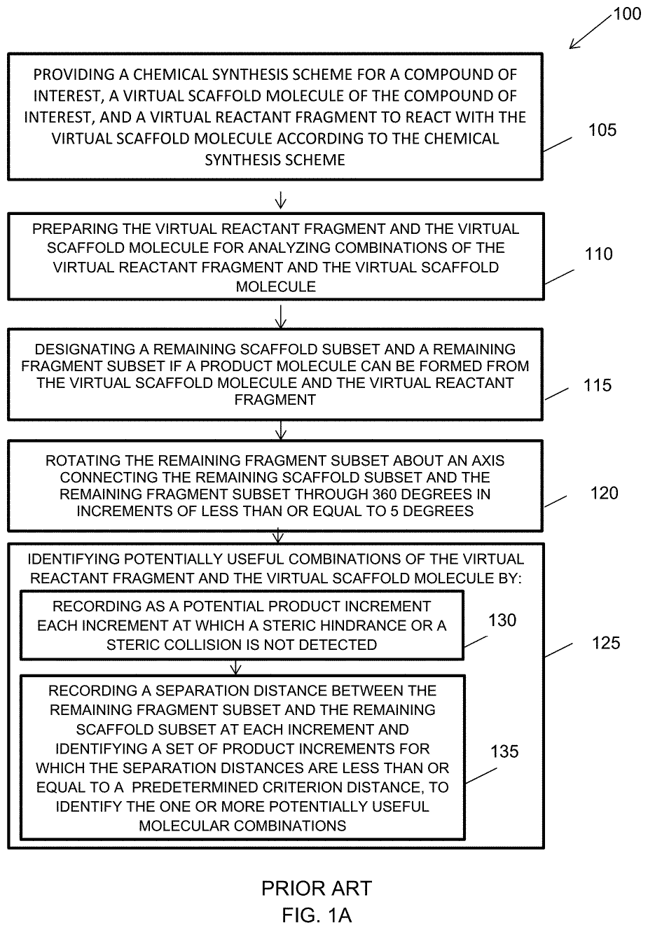

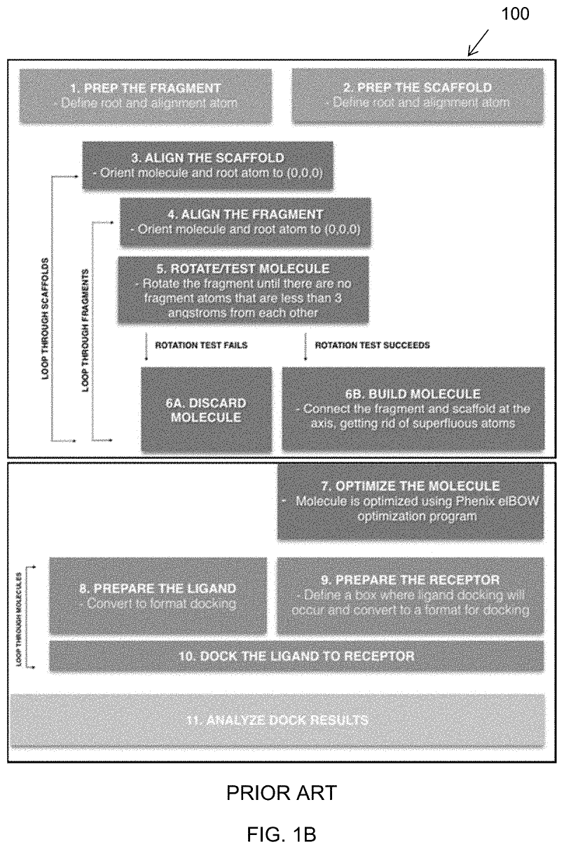

A prior art method for combinatorial in silico drug-lead optimization exists. FIG. 1 shows a flowchart for this prior art method. Prior art method 100 begins with block 105, providing a chemical synthesis scheme for a compound of interest, a virtual scaffold molecule of the compound of interest, and a virtual reactant fragment to react with the virtual scaffold molecule according to the chemical synthesis scheme. Block 110 includes preparing the virtual reactant fragment and the virtual scaffold molecule for analyzing combinations of the virtual reactant fragment and the virtual scaffold molecule. The preparing step of block 110 includes providing a three-dimensional coordinate system for the virtual reactive fragment; identifying a fragment alignment atom and a fragment root atom in the virtual reactant fragment; identifying a scaffold alignment atom and a scaffold root atom in the virtual scaffold molecule; providing a three-dimensional coordinate system for the virtual scaffold molecule and aligning the scaffold root atom with an origin and the scaffold alignment atom with an x-axis; and aligning the fragment alignment atom with the scaffold root atom; and aligning the fragment root atom with the scaffold alignment atom. Method 100 further includes block 115, which includes designating a remaining scaffold subset and a remaining fragment subset if a product molecule can be formed from the virtual scaffold molecule and the virtual reactant fragment. In block 120, the remaining fragment subset is rotated about an axis defined by the fragment root atom and the scaffold root atom through 360 degree in increments of less than or equal to 5 degrees. Block 125 includes identifying potentially useful combinations of the virtual reactant fragment and the virtual scaffold molecule, by the recording of block 130 and the recording of block 135. Block 130 includes recording as a potential product each increment at which a steric hindrance or a steric collision is not detected. A steric hindrance is a condition in which an atom of the remaining fragment subset is at a distance of ˜2.00 Å or closer to an atom of the remaining scaffold subset, excluding bond atoms. A steric collision is defined here as an approach of van der Waal's surfaces of any part of a molecule to another molecule or a part of the same molecule to a distance within which the electronic surfaces of the interacting parts resist any further approach. In block 130, recording a separation distance between the remaining fragment subset and the remaining scaffold subset at each increment and identifying a set of product increments for which the separation distances are less than or equal to a predetermined criterion distance is performed. Block 135 includes recording a separation distance between the remaining fragment subset and the remaining scaffold subset at each increment and identifying a set of product increments for which the separation distances are less than or equal to a predetermined criterion distance to identify the one or more potentially useful molecular combinations.

In the course of a typical virtual-computational or nonvirtual-wet-laboratory-based drug discovery program, initial candidate “hit” molecules are identified as molecules that may interact with and possibly inhibit or otherwise affect a given drug target (usually a cellular protein or a protein from a pathogen). The virtually identified molecules or those identified through conventional wet-lab procedures such as high throughput screens are then normally tested in wet-lab experimentation to verify their functional properties in a relevant assay that when successful, provides support for their interaction with, and effects on, the target. It is these initial hit molecules that are molecularly varied for potential identification of variant molecules that have more optimal molecular, biochemical and pharmacological properties using the prior art method 100.

The prior art method 100 (also, herein, “chemical variation procedure 100”) is used as a virtual chemical synthesis procedure in embodiments of the present invention a first time to produce thousands to millions of virtual variant candidate molecules in short periods of time, e.g., one to three days. From analyses performed by those skilled in organic chemical synthesis and/or medicinal chemistry, synthetic methods to synthesize the previously identified candidate molecule(s) are devised or elucidated from the literature. From the so-elucidated chemical synthetic pathways of the candidate molecules, a set of varied precursor molecules (combinatorial fragments) can be identified that would likely be capable of reaction to generate variants of the originally identified candidate “hit” molecule. By applying blocks 105-135 of prior art method 100, these fragments can be combined to identify a large set of thousands or millions of variant candidate molecules that are derivatives of the originally identified candidates that may possess one or more potentially useful molecular combinations.

The second set of one or more variant candidate molecules with predicted improvements are then synthesized by organic chemistry synthetic methods and tested using actual biochemical, biophysical, pharmacological, cell biological, and/or animal experimentation, including the chemical synthesis schemes identified originally identified for a compound of interest.

Another flow diagram for the prior art method 100 is shown in FIG. 1B. In block 1, inherent in the first step is the acquisition of the 3-dimensional coordinates for the reactant fragments, i.e. commercially available or synthetically approachable compounds with the proper functional—reactive groups. These precursors can be identified from databases of commercially available compounds (http://zinc.docking.org/, https://www.emolecules.com/, https://pubchem.ncbi.nlm.nih.gov/ for examples) or provided by those with sufficient chemical expertise. Smiles, SDF formatted 3D coordinate files or otherwise formatted 3D coordinate files can be converted to the required PDB type files by a number of available programs including openbabel (O'Boyle et al., 2011), cactvs (Ihlenfeldt et al., 1994; Ihlenfeldt et al., 2002), or other programs. These collections of precursor molecules can also be curated before use in subsequent chemical synthesis steps of the hit variants for desired physicochemical properties (e.g., molecular weight, polarity/nonpolarity, number of hydrogen bond acceptors and/or donors, molecular weights of the fragments, etc.) by the use of other cheminformatic programs using either experimentally derived properties or predicted or calculated properties. The cactvs tools suite from Ihlenfeldt et al., 2002 (http://www2.chemie.uni-eriangen.de/software/cactvs/tools.html) is very useful in this regard. Block 1 continues with the “marking” of the collected fragment molecules. In block 2, the ChemGen process continues with the “marking” of scaffold and precursor molecules. In one example, the scaffold could be the chloroacetyl chloride molecule and the first precursors could be a collection of amines assembled as described above. In brief, the reactive groups of the scaffold molecule and the precursors are identified via so-called marking programs that take into account what reactive atoms are eliminated during reaction (the “alignment atoms”), which atoms remain in the molecule that is produced where the new bond is formed (the “root atoms”). For proper identification of the root and alignment atoms, the nature of the element and the number of substituents that are expected to be attached to these atoms is provided by the program. While marking of simple combinations of a single scaffold with few individual reacting precursors can be performed manually, processing larger sets of scaffold or precursor molecules is facilitated by automated marking procedures. Marking is achieved by iteratively searching for atoms that fulfill the requirements for each root and alignment atom in both scaffolds and precursors that react with them. After root and alignment atoms are identified, final marking of the exact atoms occurs via writing to the beta factor value column of the PDB file of the outputted “marked” precursor molecule. A combination of scripts in bash and tcl (Ousterhout and Jones, 2010) are used to control this process with the tcl scripts processed in the Visual Molecular Dynamics tkconsole (Humphrey et al., 1996). It should be noted that for each additional chemistry used (amino group reaction with acyl chlorides, thiol reaction with alkylchlorides, etc.) unique marking scripts need to be individually and uniquely programmed. The program may be expanded to additional chemistries other than those shown here. In block 3, marking of the precursor molecules is complete, the ChemGen build process is started. Again a combination of bash scripts controlling tcl scripts in the Visual Molecular Dynamics program are used to perform these actions. In block 3, for simplicity, the root atom of the scaffold is moved to the origin of the X Y Z coordinate system and the alignment atom is oriented on the X axis. In block 4, the marked alignment atom of the incoming precursor molecule (“the fragment”) is then aligned on the scaffold root atom, and the root atom of the incoming precursor is aligned on the scaffold alignment atom. Neither alignment atoms will be present in the final product. One improvement of the present invention over the prior art are routines that insure that the bond that will be formed between remaining root atoms are of the proper bond length. Subsets of each scaffold atom and each fragment atom that remain in the product molecule are identified and, in block 5, fragment atom subsets are rotated about the root-to-root axis through 360° in small angular increments. At each increment, steric collisions of atoms are assessed and recorded. If rotation through 360° is made and no solution without steric collisions is detected, the potential product is not carried forward in the process, since these potential molecules that fail the rotation tests would likely be difficult if not impossible to synthesize (block 6A of prior art FIG. 1B). In block 6B, if increments of rotation are found that result in no steric collisions, those with the most widely separated subset groups are written to the final PDB coordinate file for the product. In block 7, optionally, the products of these syntheses can be geometrically optimized by the use of external programs like phenix.elbow (Moriarty et al., 2009), antechamber (Wang et al., 2001), GAMESS(Schmidt et al., 1993), or others. Blocks 8-10 of prior art FIG. 1B involve the conversion of the resultant variant product from block 6 (or block 7, if optimizations were performed) to a unified heavy atom model (in this case, a pdbqt format) as required by the Autodock or Autodock vina programs (Morris, Huey et al. 2009, Trott and Olson 2010), which are routinely used here to approximate interaction binding energies and to determine chemically reasonable molecular orientations of the variant molecule with the “receptor molecule”, most commonly, its target protein. It should be noted that other ligand interaction programs can be employed in addition to or in place of the Autodock programs (see for example the programs evaluated in Wang, Sun et al. 2016).

The subsequent testing of these new variants of the originally identified hit molecule using computational molecular docking programs to identify improved ligand interactions (blocks 8-11 of prior art FIG. 1B). Other cheminformatic programs can be employed to identify variants with predicted improvements in pharmaceutical properties. From among the combinatorial fragments used to produce this large set of variant candidates, a small set of novel variant candidates can be identified that have pharmacological characteristics that are predicted to be improved.

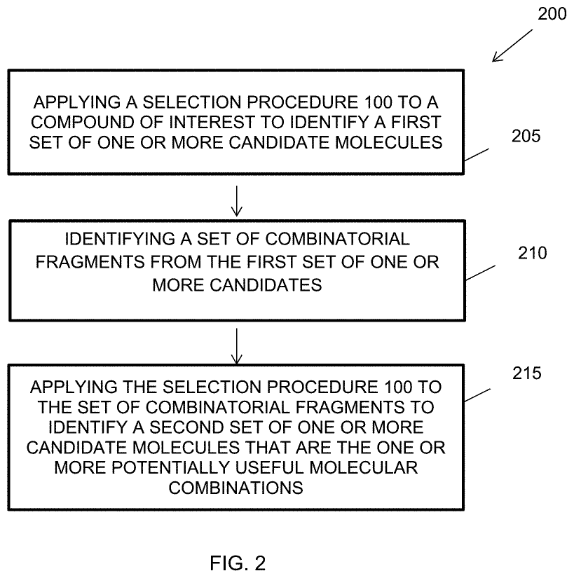

FIG. 2 shows a flowchart of the method 200 for identifying one or more potentially useful molecular combinations. In block 205, method 200 applies selection procedure 100 to a compound of interest to identify a first set of one or more candidate molecules. Block 210 of method 200 includes identifying a set of combinatorial fragments from the first set of one or more candidates. This identifying step includes the use of prior art intelligent pre-selection methods for combinatorial fragments and deep-learning and advanced computational guided decisions in predicting variant molecule characteristics such as affinity to target, toxicity, off-target interaction affinities, and likeness scores, applied to the first set of one or more candidates to identify a set of combinatorial fragments. Block 215 includes applying the selection procedure 100 to the set of combinatorial fragments to identify a second set of one or more candidate molecules that are the one or more potentially useful molecular combinations.

Prior art for one type of intelligent pre-selection of compounds in FIG. 2, block 205, that interact with a desired target protein structure and that avoid undesired protein structures is described in Brewer et al. 2014. These practitioners used a novel subtractive in silico docking method to specifically target small drug-like molecules to the nucleotide binding domains of an ABC transporter that is thought to cause multidrug resistances to chemotherapeutic agents and chemotherapy failures. Blocking this transporter with a small molecule inhibitor would be expected to be clinically useful. The method involved the determination of computationally predicted protein ligand interactions to the desired target structures on the protein using Autodock programs. The starting set of drug-like molecules was obtained from the ZINC database and contained approximately 5 million compounds. Computational analysis determined that 182,142 molecules had a desired estimated affinity to the targeted structures of the ABC transporter (its nucleotide binding domains or “NBDs”) of less than or equal to 200 nM. In the second step of this prior art, a set of “subtractive” docking calculations were performed wherein the 182,142 molecules identified in the first step were tested computationally for interactions with undesirable protein structures. (Such structures do not need to be the same protein—for example, cytochrome P450 drug metabolizing enzymes or essential kinases in the cell would be undesirable.) In this particular case, the undesirable structures were located on the same ABC transporter and included the drug transport structures of the protein (drug binding domains, or “DBDs”). Binding of putative inhibitors of the ABC transporter to the DBDs was deemed undesirable, because previous clinical testing of inhibitors of this ABC transporter had all failed and one of the characteristics of these failed molecules were that the inhibitors were almost all transported out of the targeted cancer cell by the ABC transporter itself. Identifying molecules that do not interact with the transport structure of the transporter, and thereby are not themselves transport substrates of the transporter, was deemed by the practitioners as very desirable. After applying the second docking calculations to the first set of molecules that bound tightly to the nucleotide binding domains of the ABC transporter, any molecule that did not differ in estimated affinity to the DBDs compared to the NBDs by at least a factor of 200 were rejected and not further considered. This left a set of approximately 250 molecules that were predicted to preferentially bind to the NBDs of the ABC transporter relative to the drug transporting structures. Brewer et al. 2014 acquired 35 of these molecules and tested them for inhibition of ATP hydrolysis and inhibition of ATP-analog binding to biochemically isolated ABC transporter. Four molecules of the 35 tested (˜11%) showed the desired characteristics of inhibiting the power utilization steps of the transporter (ATP hydrolysis) and three inhibited ATP-analog binding to the protein. In additional tests performed by Follit et al. 2015 three of these four molecules were shown to reverse the multidrug resistant phenotype of a highly multidrug resistant human prostate cancer cell line. In 2018, Nanayakkara et al. showed that these three molecules reversed multidrug resistance in a human ovarian cancer cell line. Nanayakkara et al. 2018 also importantly directly showed that none of these three molecules were transport substrates of the ABC transporter which strongly validated the “subtractive” docking methods applied originally by Brewer et al. 2014 (i.e. inhibitors of the targeted protein were identified that reversed multidrug resistances in cancer cell line, but none of these molecules was transported itself by the ABC transporter protein). The success rate for identification of molecules with the desired biological activities (inhibiting the ABC transporter, reversing multidrug resistances in cancer cells, and not being good transport substrates of the targeted ABC transporter) was 3 in 35 for this study (9/).

The type of intelligent pre-selection of compounds of interest as described in the prior art of Brewer et al. 2014, Follit et al. 2015 and Nanayakkara et al. 2018 as well as other selection schemes can be used with the prior art described in FIGS. 1A and B to efficiently optimize initially identified compounds in their pharmacological properties (FIG. 2). The subtractive docking methods used by Brewer et al. 2014 can be applied to initially identify compounds that inhibit or otherwise preferentially interact with a given target protein structure and optionally that avoid an undesirable structure or structures (block 205, FIG. 2). These initially identified compounds (commonly referred to as “hit” compounds) rarely have the characteristics required for application in clinical settings and need to undergo the “lead optimization” studies discussed above. Application of the ChemGen prior art (FIGS. 1A and B) can be utilized to create in silico many different structurally related variants of these initial hit molecules (block 210, FIG. 2). The prior art described in block 215 of FIG. 2 would represent a second application of the intelligent selection methods to the generated variants from the ChemGen prior art to identify variant compounds with improved characteristics (for example increased affinity to the targeted protein structures).

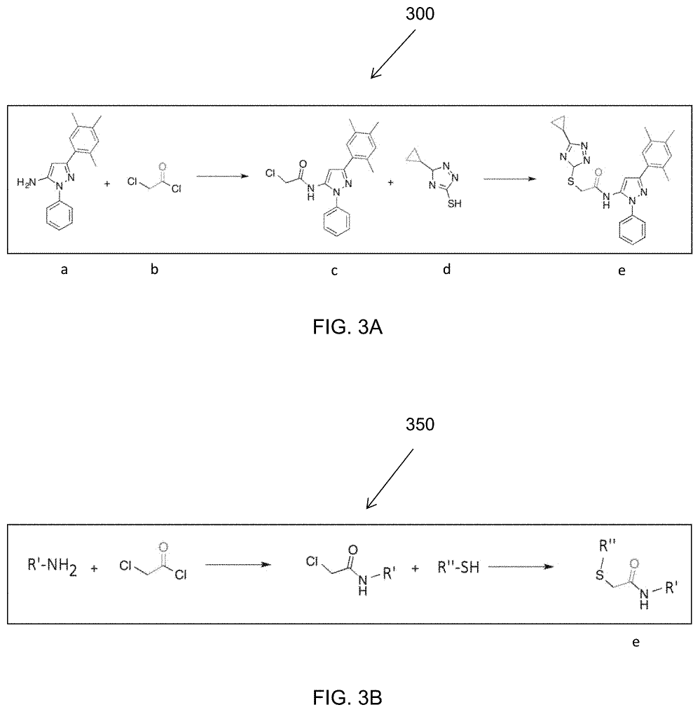

The present invention, illustrated in a method embodiment 200 in FIG. 2, is distinguishable from prior art method 100 of FIGS. 1A and 1B, and the ChemGen prior art described herein because the combination of these familiar elements produces an improvement that is much more than the predictable use of the prior art elements according to their established functions. In a reduction to practice, the combination of these elements led to the identification of variant molecules of the inhibitor of the ABC transporter compound 29 from Brewer et al. 2014 (FIG. 2 block 205 and as described above), which the ChemGen prior art is applied as described above to the results of block 205 (FIG. 2, block 210, as chemically varied in FIGS. 3A and B), and then intelligently selected as in Brewer et al. 2014 a second time (FIG. 2, block 215, and as described above), which were then chemically synthesized and biochemically and cell biologically tested, possessed all of the desired characteristics (inhibiting the ABC transporter, reversing multidrug resistances in cancer cells, and not being good transport substrates of the targeted ABC transporter) with improved pharmacological characteristics at a success rate of 100% (five out of five synthesized molecules passed all tests and all five had a higher affinity to the targeted protein than did the original hit molecule, compound 29). This is in contrast to the success rates observed by Brewer et al. 2014 as tested also by Follit et al. 2015 and Nanayakkara et al. 2018 of ˜9%, an improvement in overall success rates when applied to identifying optimized lead compounds with desired characteristics of over 11-fold. When compared to conventionally modified molecular structures (i.e. visual inspection for optimization by a skilled and experienced organic chemist—a rational design approach), followed by chemical synthesis of these compounds, a success rate for identification of variants of compound 29 with improved characteristics of maximally 20% was observed. In the latter study, five compounds were varied, synthesized and tested, four of which were converted into transport substrates of the ABC transporter (an undesirable characteristic), while only one showed improved affinity to the ABC transporter while retaining the other advantageous characteristics. The surprising results of the present invention, that the combination of prior arts methods of selection, variation by ChemGen, and reselection (FIG. 2) versus conventional rational design considerations led to a 5-fold better identification of lead optimization variants with improved properties is striking and unexpected. See the example detailed herein.

In the selection procedure 100 used in embodiments of the present invention, preparing the virtual reactant fragment and the virtual scaffold molecule for analyzing combinations of the virtual reactant fragment and the virtual scaffold molecule includes providing a three-dimensional coordinate system for the virtual reactive fragment. This may be done, for example, by identifying a three-dimensional coordinate system from a database of commercially available compounds such as zinc.docking.com or www.emolecules.com. SMILES-formatted or SDF-formatted 3D coordinate files for a virtual reactive fragment can be converted to PDB-type files by a program such as the openbabel program (O'Boyle et al. 2011) or the cactvs program (Ihlenfeldt et al. 2002; Ihlenfeldt et al. 1994).

In the selection procedure 100 used in embodiments of the present invention, preparing the virtual reactant fragment and the virtual scaffold molecule for analyzing combinations of the virtual reactant fragment and the virtual scaffold molecule also includes marking, via one or more marking programs, the virtual reactant fragment and the virtual scaffold molecule: identifying an alignment atom and a root atom in each of the virtual reactant fragment and the virtual scaffold molecule. In each case, a root atom is an atom that remains after the reaction and an alignment atom is an atom that does not remain after the reaction. A combination of bash (main_new10.bsh) and tcl (various generic_*.tcl) scripts running a program such as Visual Molecular Dynamics (Humphrey et al. 1996) may be used to mark the virtual reactant fragment and the virtual scaffold molecule. Using these tools, the alignment and the root atoms are marked in the beta column of respective 3D coordinate files. Each chemistry step which is inherent in marking is specifically programmed. This programming allows the selection procedure and embodiments of the present invention to go beyond the pre-specified “click” chemistries that limit some other prior art methods, but that do not limit the present invention.

In the selection procedure 100 used in embodiments of the present invention, preparing the virtual reactant fragment and the virtual scaffold molecule for analyzing combinations of the virtual reactant fragment and the virtual scaffold molecule further includes moving the root atom of the virtual scaffold molecule to the origin of the x,y,z coordinate system assigned to that molecule and orienting the alignment atom of the virtual scaffold molecule to the x-axis of that coordinate system. This preparation also includes aligning the alignment atom of the virtual reactant fragment with the root atom of the virtual scaffold molecule and aligning the root atom of the virtual reactant fragment with the alignment atom of the virtual scaffold molecule. A combination of bash and tcl scripts running a program such as Visual Molecular Dynamics may be used to prepare the virtual reactant fragment and the virtual scaffold molecule as described herein.

A virtual scaffold molecule of a compound of interest is a computer model of a molecule such as SMU 29 or SMU 45. A virtual reactant fragment is a computer model of a fragment that can be reacted with a compound of interest. The prior art method 100 and the prior art method 150 make use of modeling a reaction of the virtual reactant fragment with the virtual scaffold molecule according to the chemical synthesis scheme.

In the selection procedure 100 used in embodiments of the present invention, at each increment, steric hindrances of atoms may be assessed and recorded. If rotation through 360 degrees is completed and no solution without a steric hindrance is detected, a product of the remaining scaffold subset and the remaining fragment subset and is identified as failed. If increments of rotation are found that result in no steric hindrances, a separation distance between the remaining fragment subset and the remaining scaffold subset at each such increment is recorded and a set of product increments for which the separation distances satisfy one or more predetermined criteria is identified. For example, the increments with the most widely separated subset groups may be determined as preferred and identified as a preferred set of product increments.

Alternatively, in the selection procedure 100 used in embodiments of the present invention, at each increment, steric collisions of atoms may be assessed and recorded. If rotation through 360 degrees is completed and no solution without a steric collision is detected, a product of the remaining scaffold subset and the remaining fragment subset and is identified as failed. If increments of rotation are found that result in no steric collisions, a separation distance between the remaining fragment subset and the remaining scaffold subset at each such increment is recorded and a set of product increments for which the separation distances satisfy one or more predetermined criteria is identified. For example, the increments with the most widely separated subset groups may be determined as preferred and identified as a preferred set of product increments.

A virtual scaffold molecule of a compound of interest is a computer model of a molecule such as SMU 29 or SMU 45. A virtual reactant fragment is a computer model of a fragment that can be reacted with a compound of interest. The selection procedure used in embodiments of the present invention makes use of modeling a reaction of the virtual reactant fragment with the virtual scaffold molecule according to the chemical synthesis scheme.

In the selection procedure 100 used in embodiments of the present invention, analyzing combinations of the virtual reactant fragment and the virtual scaffold molecule includes identifying a remaining scaffold subset and a remaining fragment subset if a product molecule can be formed from the virtual scaffold molecule and the virtual reactant fragment. Then, the virtual scaffold molecule and the virtual reactant fragment are rotated about each other in increments around an axis defined by the root atoms of each virtual structure.

Further, the selection procedure 100 used in embodiments of the present invention uses the predicted binding energies and predicted fold improvements (defined as the ratio of the estimated K_{d} of the compound of interest to the predicted K_{d} of candidates for synthesis and testing) as factors in assessing the suitability of candidates for synthesis and testing.

FIGS. 3A and 3B show a chemical synthesis scheme 300 for SMU 29 from Brewer et al. (2014). By employing many different amino and thiol precursor compounds, many derivatives of SMU 29 can be produced. FIG. 2A shows chloroacetyl chloride (FIGS. 3A-B) reacting with an amine (FIG. 3A) to produce a 2-chloroacetamide derivative 315 (FIGS. 3A-C). The 2-chloroacetamide derivative (FIGS. 3A-C) is then reacted with a thiol compound (FIGS. 3A-D) produce the compound of interest (FIGS. 3A-E), SMU 29, via the scheme of FIG. 3B. FIG. 3B illustrates a generalized synthesis scheme 350 for the compound of interest (FIGS. 3A-E), SMU 29. Various amines and thiol compounds can be reacted to create variants of the SMU 29 structure.

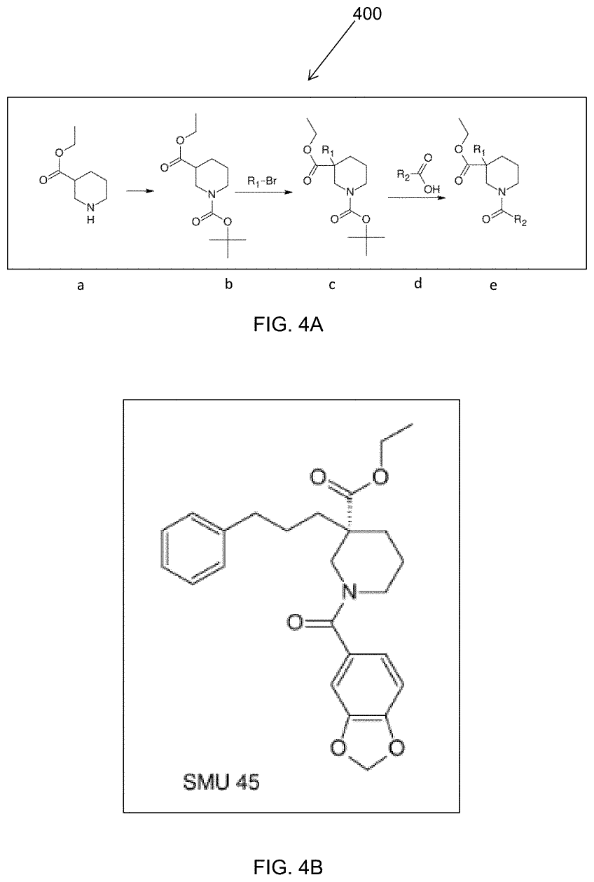

FIG. 4A shows a chemical synthesis scheme 400 for the compound of interest (FIGS. 4A-E), SMU 45, from Brewer et al. 2014. By employing many different bromo- and carboxylic acid derivatives, many novel SMU 45 derivatives can be produced. To produce the compound of interest (FIGS. 4A-E), SMU 45, the ring nitrogen of ethyl piperidine-3-carboxylate (FIG. 4A) is first protected and then reacted with a bromo-compound (FIGS. 4A-B), producing the ethyl 3-(3-R₁-derivitized)piperidine-3-carboxylate (FIGS. 4A-C). This compound is then reacted with carboxylic acid derivatives or acyl chloride derivatives (FIGS. 4A-D) to form the ethyl 1-(R₂-derivitized)piperidine-3-carboxylate. FIG. 3B shows the compound of interest (FIG. 4A-e), SMU 45.

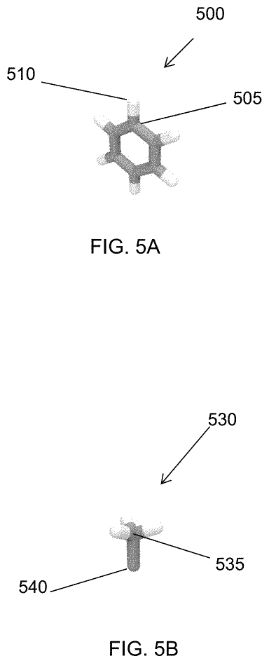

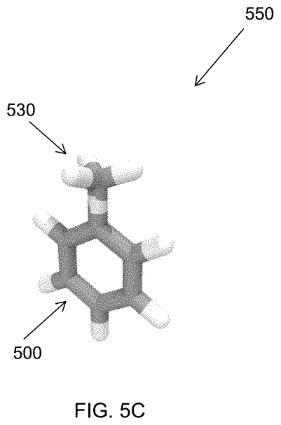

FIGS. 5A, 5B, and 5C illustrate steps in preparing a virtual reactant fragment and a virtual scaffold molecule for analyzing combinations. For ease of illustration, a relatively simple molecule, benzene, is illustrated as the virtual scaffold module and a methyl group is illustrated as the virtual reactant fragment. FIG. 5A shows a virtual scaffold molecule, a benzene molecule 500, with root atom 505 and alignment atom 510. FIG. 5B shows a virtual reactant fragment, a methyl group 530 with root atom 535 and alignment atom 540. FIG. 5C depicts the combination 550 of the benzene molecule 500 and the methyl group 530.

Where a chemical synthesis scheme has one or more intermediate steps, as, for example the scheme for SMU 29 has, the method of the present invention can be repeated as necessary. Three-dimensional coordinates for a virtual reactant fragment for an intermediate step may be obtained as described herein. This intermediate virtual reactant fragment and the last product from the last analysis, as the intermediate virtual scaffold molecule of the intermediate compound of interest, can be prepared and analyzed as described herein. Potentially useful combinations of the intermediate virtual reactant fragment and the intermediate virtual scaffold molecule can be identified as described herein.

The skilled artisan will recognize that methods and systems of the present invention allows the synthesis of large numbers of variants in silico to inform choices of which of the vast numbers of possible compounds to synthesize for experimentation, saving expense and time in the drug development process.

Example of Use of an Embodiment of the Present Invention.

Resistances of cancers to chemically unrelated anti-cancer drugs are frequently caused by the expression of members of the ABC transporter superfamily, including ABCB1 (P-glycoprotein or P-gp)¹⁻³ and/or ABCG2 (the breast cancer resistance protein or BCRP)^{3, 4}. The phenomenon of multidrug resistance (MDR) remains a major obstacle in the treatment of both adult and pediatric cancers⁵⁻⁷. Despite close to 40 years of intense research, no inhibitor of these proteins has yet been approved for clinical use⁸⁻¹³. The reasons for failure in clinical trials are multifaceted: some could be attributed to flaws in trial design, others reported tumor penetration problems, and still others failed because of drug-drug interactions and associated toxicities. A significant number of failures of the clinical trials may be attributed to the fact that many of the assessed MDR-inhibitors were also good transport substrates of the pumps. This latter characteristic likely required elevated systemic inhibitor concentrations for efficacy that may have resulted in significant off-target toxicities. The fact that a Phase III trial using the immunosuppressant, cyclosporine A, led to improved patient outcomes in poor-risk acute myeloid lymphoma patients¹⁴ suggests, however, that despite the limited success in finding clinically successful inhibitors of MDR pumps, these proteins are important targets for drug discovery and development.

One of the biggest obstacles to developing effective inhibitors of membrane proteins like P-gp and BCRP has been the dearth of detailed structural and mechanistic knowledge, the lack of which made targeting these pumps ineffective. Over the last several years, however, significant advances in knowledge of the structure¹⁸⁻²⁰ and mechanism²¹⁻²⁵ of P-gp, BCRP and other related pumps have emerged that will enable the design of potent inhibitors of the pumps that may prove to be more successful in clinical applications.

Using the evolutionary relationship of different ABC transporters and the structural knowledge of both prokaryotic and eukaryotic ABC transporters, dynamic models of human P-gp 2 were created and a putative catalytic cycle²² was simulated that correlated well with published biochemical and biophysical studies as well as with the recently elucidated outward facing structure of the human P-gp¹⁶. These conformationally dynamic models of human P-gp in ultrahigh throughput in silico screenings were previously used to identify and characterize inhibitors of P-glycoprotein²⁶. One desirable characteristic of the inhibitors identified in these screens was that these potential P-gp inhibitors should not be transport substrates of the pump²⁶. For this reason, drug-like molecules that were computationally predicted to interact well with the nucleotide binding domains were computationally counter-screened for interactions with the drug binding parts of the protein. Compounds were discarded from further evaluation, if significant binding to the drug binding sites was predicted by the in silico docking calculations²⁶. Using this approach, three compounds were initially identified and characterized that reversed the multidrug resistance phenotype of various cancer cell lines in both conventional and microtumor-spheroid cell cultures²⁶⁻²⁸. The efficacy of the compounds was assessed using P-gp overexpressing, multidrug resistant prostate and ovarian cancer cells in culture. All three compounds were observed to reverse multidrug resistance by increasing lethality of various chemotherapeutics. The increased lethality was correlated with increased cellular retention of the chemotherapeutics when inhibitor was present²⁸. Importantly, studies also showed that these three P-gp inhibitors were not significantly transported by P-gp²⁸, supporting the premise that these inhibitors would not bind effectively to the drug binding domains of the pump²⁶.

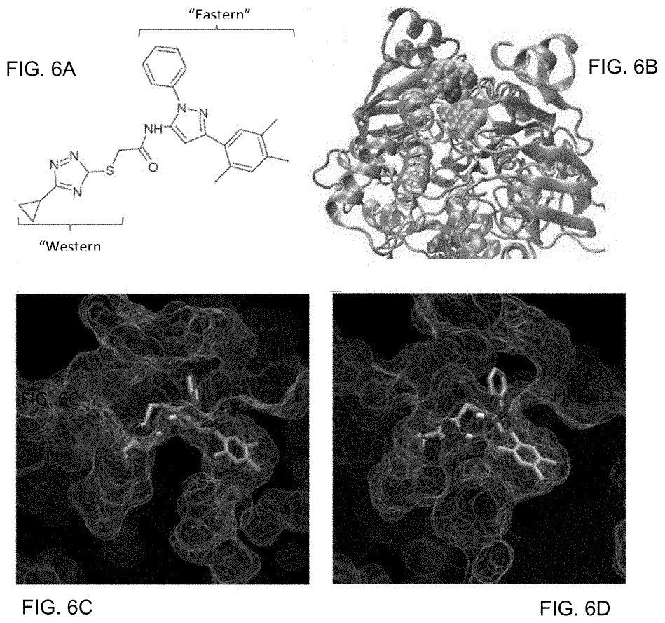

One of these compounds that reversed MDR phenotypes in cancer cells was predicted to be an allosteric inhibitor of P-glycoprotein (“SMU-29”, “compound 29”, or “29” herein, 2-[(5-cyclopropyl-4H-1,2,4-triazol-3-yl)sulfanyl]-N-[2-phenyl-5-(2,4,5-trimethylphenyl)-pyrazol-3-yl, FIG. 6A.)²⁶. The presence of compound 29 caused increased penetration of a P-gp pump substrate into microtumors of a highly P-gp overexpressing prostate cancer²⁸ and co-administration of 29 with a chemotherapeutic resulted in increased cell death via apoptotic mechanisms and resulted in tumor-spheroid size reduction²⁸. The binding pose of 29 docked at the highest affinity interaction site on P-gp as observed in the original study²⁶ is presented in FIG. 6B. This site is located in the N-terminal half of the protein near the interface of the two nucleotide binding domains and is significantly outside of the ATP binding sites. The computational prediction that 29 acted as a potential allosteric inhibitor of P-gp ATP hydrolysis was supported by the observation that 29 did not affect the binding of an ESR active analog of ATP (SL-ATP, 2′,3′-(2,2,5,5,-tetramethyl-3-pyrroline-1-oxyl-3-carboxylic acid ester ATP) to P-gp²³, while three other compounds assessed in that same study inhibited SL-ATP binding to P-gp. In contrast to compound 29, these latter three compounds had been predicted by the computational studies to partially overlap with the ATP binding sites of the transporter²⁶. They were therefore anticipated to be competitive inhibitors of ATP binding. All four compounds inhibited both basal and transport-substrate stimulated ATP hydrolysis activities of purified P-gp²⁶, suggesting direct interaction with the energy harvesting steps of substrate pumping by P-gp.

Inspection of the computational docking of 29 at the putative allosteric site on P-gp (FIGS. 6C and 6D) indicated that considerable space was available for additional interactions between the protein and some parts of compound 29, especially around what is called herein the “Western” end of the inhibitor (see FIGS. 6A, 6C, and 6D). In contrast, the “Eastern” end of 29 docked to P-gp was partly exposed to the external surface of P-gp (the phenyl group at the top of FIG. 6B) while the trimethylphenyl group seemed to nearly optimally fit into a cavity in P-gp (FIGS. 6B, 6C, and 6D).

A number of computational approaches exist that seek to analyze the potential chemical space of a hit compound by creating virtual libraries of variants of a given hit molecule²⁹⁻³². These approaches mostly vary in the chemistry that is applied to perform the synthesis reactions. Presented herein are initial efforts at creating variants of compound 29 with optimized binding affinity to P-glycoprotein using a novel computer-aided and structure-based approach that was applied to the “Western” end of compound 29. Results of the virtual synthesis of a moderate number of variants of 29 and the virtual screening of these variants with structural models of P-gp are reported. A small portion of the nearly infinite chemical space around hit compound 29 was synthesized and assessed for reversal of the MDR phenotype in a multidrug resistant prostate cancer cell line that over-expresses P-gp (DU145TXR³³). Using the same cell line, the inhibition of P-gp-catalyzed pumping of a P-gp substrate by these 29 variants was also assessed. In addition, biochemical analysis of the mode of P-gp inhibition was performed for all 29-variants using ATP hydrolysis and ATP binding assays as in²⁶. After initial evaluation of the computationally predicted inhibitor variants in these assays as well as of the physicochemical properties of these variants, we developed a new, structure-based rational design to synthesize and analyze a small number of 29-derivatives with different structural and physicochemical characteristics. These compounds were not initially computationally evaluated using the subtractive binding routines as described in²⁶, but were chosen mostly for the shape and size of the Western half of the molecule as well as for their physicochemical characteristics like polar surface area and solubility. All of the novel 29-variants were experimentally assessed for their potential of being transported by P-gp. The work led to the discovery of several variants of P-gp inhibitor hit compound 29 with improved efficacy in reversing MDR in P-glycoprotein over-expressing cancer cells by inhibiting P-gp catalyzed substrate pumping.

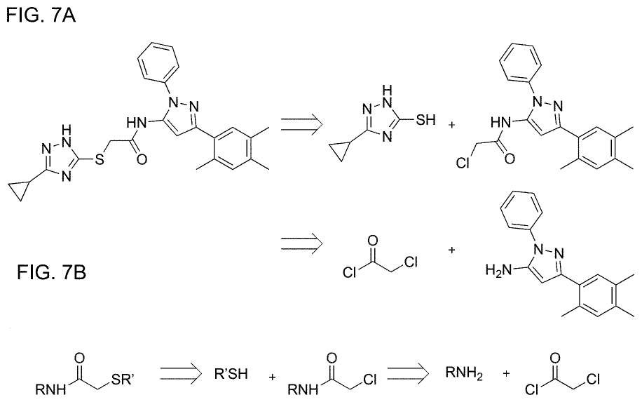

Results. Virtual synthesis of novel variants of the “Western” half of SMU-29 using the ChemGen computational suite. Evaluation of the fit of compound 29 into a putative allosteric binding site on P-gp as visualized from the results of docking studies the inventors recognized that if variants of inhibitor 29 were made larger and more hydrophobic they would likely fill the relatively large hydrophobic pocket in the protein where the cyclopropyl group of 29 interacts (FIGS. 6C and 6D). Substitutions at the “western” (cyclopropyl) end of the molecule were therefore deemed most promising. The inventors further recognized that some of these variants might potentially bind with increased affinity to the protein because of the added protein—ligand interactions. In order to test these solutions, a central retrosynthetic disconnection of a carbon-sulfur bond in compound 29 (FIG. 7A) was generalized for virtual syntheses (FIG. 7B). The ChemGen program was used to virtually synthesize a number of variants of compound 29 that had different substituents at the “Western” thiol-derived part of the molecule. Several thousand sulfur-containing compounds were collected from the ZINC database³⁴ by a simple search for carbon-sulfur single bonds. After pruning this set of compounds for those that could be converted to thiols using the ChemGen precursor marking procedures as described herein, 647 thiol-containing molecules remained in the set. These thiol precursors were then used in the final reaction step of the retrosynthesis as shown in FIG. 7B. The choice of the thiol precursors was left unbiased. It was assumed that if a derivative proved interesting in subsequent in silico screening experiments, precursor synthesis would be feasible since the parent compounds were commercially available. The scaffold compound used to react with these thiols was 2-chloro-N-[1-phenyl-3-(2,4,5-trimethylphenyl)-1H-pyrazol-5-yl]acetamide, which includes the “eastern” part of compound 29 (FIG. 6A). Virtual syntheses of the resulting 647 variants of compound 29 were performed using ChemGen applied to the single scaffold and thiol precursors as described herein. Final geometrical optimization of the virtually synthesized molecules was performed using the phenix.elbow³⁵ program.

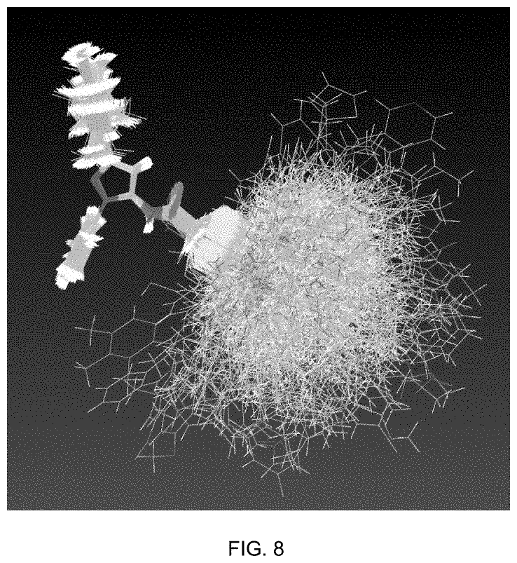

Some of the potential chemical space of these 647 ChemGen-produced “Western” variants of 29 (“Group 1” compounds) is visualized in FIG. 8. FIG. 8 shows the 647 virtual 29-variants aligned on the heavy atoms of the common “Eastern” 1-phenyl-3-(2,4,5-trimethylphenyl)-1H-pyrazol-5-yl group. The geometrically optimized virtual molecules were loaded in the Visual Molecular Dynamics program and superposed using the RMSD Trajectory Tool. It should be noted that the superposition of these atoms is not perfect because the geometric optimizations used to produce the molecules induced slight variations in the rotatable bonds that are present in this part of the molecule. FIG. 8 shows that the chemical space sampled by these molecules is relatively large even though only 647 molecules were produced. It should also be noted that the space visualized in this representation underestimates the actual chemical space of these molecules since rotations of single bonds in the varied substituents (not shown) would fill even more of the potential volume.

Docking to P-glycoprotein and chemical synthesis of compound 29 variants. The 647 Group 1 molecules created by ChemGen were used in docking studies to the same structural model of P-gp that was employed previously and led to the identification of compound 29 as a potential inhibitor²⁶ (see steps 8 through 10 of FIG. 1B, the “in silico docking routine”). Docking was performed as in reference²⁶ but used a target box that encompassed the putative allosteric site on P-gp (FIG. 6B) instead of the larger target box described in reference²⁶. The original unmodified compound 29 was included in these calculations so that the estimated binding affinities calculated by the docking software for each of the variants could be compared to the affinities estimated for parental compound 29. It was assumed that this type of relative comparison would allow judgments to be made on which molecules might demonstrate increased binding affinities in subsequent validation experiments using the actual compounds. Sixty-seven of these Group 1 variants of compound 29 were identified in these calculations that were predicted to interact with the putative allosteric site on P-gp more strongly than the parental 29. Importantly, since it was desirable to identify variants that are not pump substrates for P-gp, these 67 variants of 29 were then docked in a second step and counter-screened for low predicted interaction affinities with the drug binding domains of P-gp (also as described in reference²⁶). Eleven of the promising hit variants had molecular weights less than 600 Da and ratios of the estimated K_{d} of compound 29/estimated K_{d} of 29-variant that were greater than 10 (Table 1), suggesting that these variants might have higher affinity to P-gp than the original 29.

TABLE 1
ChemGen/docking routine identified variants of P-gp inhibitor 29.

| | | | | | | Topological | |
|---|---|---|---|---|---|---|---|
| | | | | ratio | | polar | |
| | | estimated | estimated | K_{d} 29/ | Molecular | surface | |
| | Synthesized | ΔG_{binding} | K_{d} | K_{d} | weight | area | Consensus |
| Variant name | variant | (kcal/mol) | (nM) | variant | (Da) | (Å²) | logP |
| opt_0009_19 | | −12.4 | 0.8 | 50 | 599 | 173.8 | 3.4 |
| opt_0005_53 | 29-541 | −12.0 | 1.6 | 25 | 522 | 132.5 | 3.9 |
| opt_0009_29 | 29-216 | −11.8 | 2.3 | 17 | 558 | 89.3 | 6.6 |
| opt_0010_33 | | −11.8 | 2.3 | 17 | 576 | 151.5 | 3.8 |
| opt_0005_47 | | −11.8 | 2.3 | 17 | 591 | 138.6 | 4.3 |
| opt_0000_30 | | −11.7 | 2.7 | 15 | 541 | 157 | 3.4 |
| opt_0002_34 | | −11.7 | 2.7 | 15 | 579 | 124 | 4.7 |
| opt_0005_27 | | −11.7 | 2.7 | 15 | 584 | 131.4 | 4.4 |
| opt_0010_49 | 29-551 | −11.6 | 3.2 | 13 | 627 | 114.2 | 5.9 |
| opt_0009_33 | 29-231 | −11.5 | 3.8 | 11 | 535 | 101.3 | 5.7 |
| opt_0004_12 | 29-227 | −11.5 | 3.8 | 11 | 587 | 92.5 | 6.4 |
| opt_0006_13 | | −11.5 | 3.8 | 11 | 596 | 130.1 | 4.6 |
| ZINC08767731 | 29 | −10.1 | 40 | 1 | 461 | 113.8 | 4.0 |

Estimated K_{d} values for the ligand interactions with P-gp were calculated from the lowest estimated binding ΔG values from the AutoDock calculations. The ratio of K_{d} values is shown as a relative value for increased affinity exhibited by the respective variants over the parent P-gp inhibitor compound 29. Molecular weights, topological polar surface areas, and consensus log P values were calculated at the SwissADME website (http://www.swissadme.c/) as described herein.

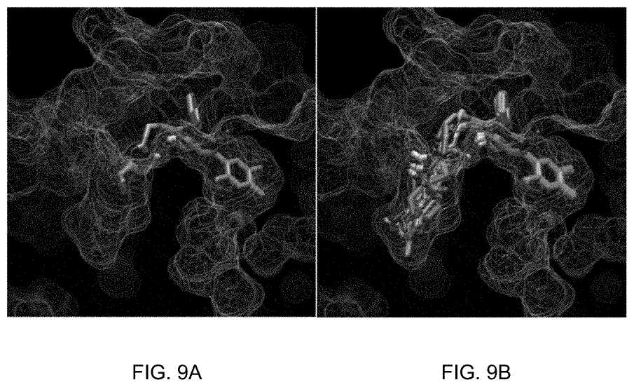

One additional compound (29-551) which contains abromo-substituent and has a molecular weight of 627 Da was added for consideration. FIG. 9A shows compound 29 and FIG. 9B shows an overlay of the 12 variants shown in Table 1 in the putative allosteric site on P-gp. It can be seen from FIGS. 9A and 9B that the ChemGen synthesis and subsequent docking routines used here generated and identified several variant compounds that were predicted to better fill the void in the putative allosteric site on P-gp than the parental compound 29 did. Docking calculations also suggested that these variants might show improvements in binding affinities to P-gp. A small number (five) of these Group 1 ChemGen-produced compounds (Table 1, 29-216, -227, -231, -541 and -551) were selected for chemical synthesis and subsequent testing for potentially improved efficacy in cancer cell culture. At this stage, no consideration was given to “drug-likeness” of the chosen compounds except for trying to keep the relative molecular weight as low as possible. Instead, the choice for synthesis of these five particular compounds was made on the basis of ease of synthesis and commercial availability of the precursor molecules.

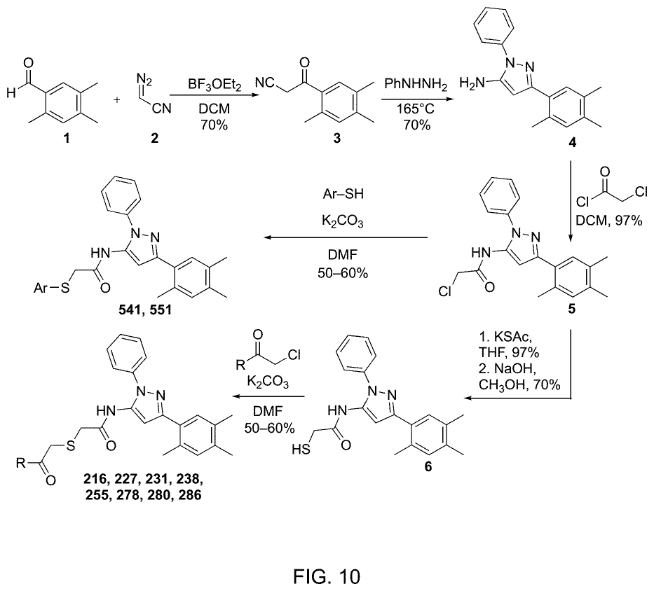

Syntheses were performed using the scheme shown in FIG. 10. The virtual retrosynthetic scheme shown in FIGS. 7A and 7B was slightly modified so that the conserved pyrazole “eastern” half would be formed from a thiol precursor. Lewis acid catalyzed reaction of aldehyde 1 with diazoacetonitrile 2 provided the α-cyanoketone 3³⁶. The core pyrazole motif was formed through condensation of 3 with phenylhydrazine, providing aminopyrazole 4 in good yield³⁷. After nucleophilic acyl substitution with chloroacetyl chloride, the thiol was formed in two steps by substitution with potassium thioacetate followed by thioester hydrolysis³⁸. With this thiol in hand, S_{N}2 reaction with various alkyl chlorides provided a modular approach to diverse derivatives of compound 29. While most of the synthesized derivatives of compound 29 consisted of an amide on the “western” half, derivative 29-216 (216) required a ketone. The target α-chloro ketone was prepared by Friedel-Crafts acylation of fluorene with chloroacteyl chloride³⁹. The resultant chloride was then subjected to a similar sequence as described above to form the thiol after nucleophilic substitution with thioacetate. The aromatic sulfide compounds 541 and 551 were prepared by substituting the alkyl chloride 5 with the respective aromatic thiols. Details of the syntheses and product analyses are provided herein.

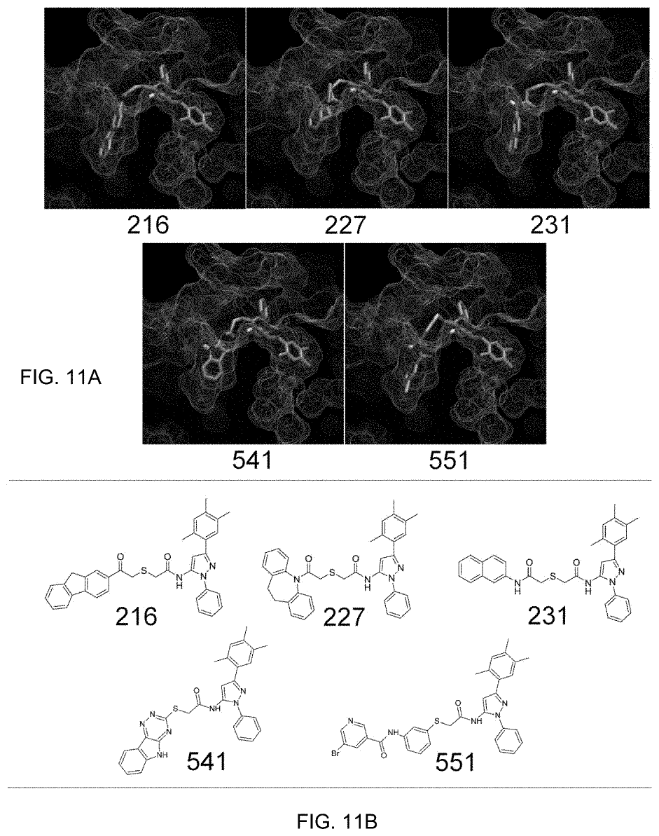

FIG. 11A shows the structures of Group 1 variants 29-216 (216), 29-227 (227), 29-231 (231), 29-541 (541) and 29-551 (551) in the highest estimated affinity docking pose (shown as licorice and colored by atom type) in the putative allosteric site of P-gp. FIG. 11B shows the chemical structures of the 29 variants underneath the respective docking images. The nearly identical binding of the “eastern” portions of compound 29, and the ChemGen/docking routine generated 29-variants is also clearly visible in FIG. 9A. FIG. 9B displays the superposition of the docking poses of all 12 of the 29 variants shown in Table 1.

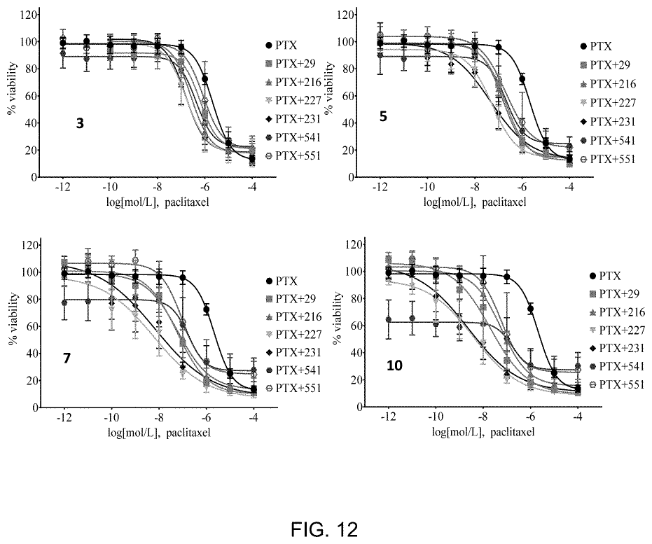

The Group 1 ChemGen/docking routine produced variants of P-gp inhibitor 29 resensitize a multidrug resistant, P-gp overexpressing prostate cancer cell line to paclitaxel. The mitochondrial reduction potential of cells is often used as an indicator for cell viability using MTT assays⁴. Using these assays it was observed that the five Group 1 derivatives of 29 predicted through the ChemGen/docking routine and synthesized here, 216, 227, 231, 541 and 551, re-sensitized the P-gp overexpressing prostate cancer cell line, DU145TXR³³, to the chemotherapeutic, paclitaxel (FIG. 12, showing results of assays at concentrations of 3 μM, 5 μM, 7 μM, and 10 μM). Analyses of the data (Table 2) revealed that in the presence of 3 μM inhibitor variant 216 the observed IC₅₀ value of paclitaxel was decreased by about 2.4 fold when compared to the presence of the parental compound 29 and about 8-fold when compared to paclitaxel alone.

TABLE 2
Increased toxicity of paclitaxel to DU145TXR in the presence of P-gp inhibitors identified by the ChemGen/docking routine.

| | Resensitization to paclitaxel with the indicated treatment and fold increased sensitivity in the presence of inhibitors | | | | | | | | |
|---|---|---|---|---|---|---|---|---|---|
| | PTX | PTX + 29 | | | PTX + 216 | | | | |
| | alone | | | Fold | | | Fold | PTX + 227 | |
| | IC₅₀ | IC₅₀ | Fold | vs | IC₅₀ | Fold | vs | IC₅₀ | Fold |
| Inhibitor | PTX | PTX | vs | PTX + | PTX | vs | PTX + | PTX | vs |
| concentration | (nM) | (nM) | PTX | 29 | (nM) | PTX | 29 | (nM) | PTX |
| 3 μM | 2120 | 629 | 3 | 1 | 266 | 8 | 2.4 | 164 | 13 |
| 5 μM | 2120 | 194 | 11 | 1 | 154 | 14 | 1.3 | 52 | 41 |
| 7 μM | 2120 | 59 | 36 | 1 | 62 | 34 | 1 | 6 | 353 |
| 10 μM | 2120 | 21 | 101 | 1 | 57 | 37 | 0.4 | 4 | 530 |

| | Resensitization to paclitaxel with the indicated treatment and fold increased sensitivity in the presence of inhibitors | | | | | | | | | |
|---|---|---|---|---|---|---|---|---|---|---|
| | PTX + 227 | PTX + 231 | | | PTX + 541 | | | PTX + 551 | | |
| | Fold | | | Fold | | | Fold | | | Fold |
| | vs | IC₅₀ | Fold | vs | IC₅₀ | Fold | vs | IC₅₀ | Fold | vs |
| Inhibitor | PTX + | PTX | vs | PTX + | PTX | vs | PTX + | PTX | vs | PTX + |
| concentration | 29 | (nM) | PTX | 29 | (nM) | PTX | 29 | (nM) | PTX | 29 |
| 3 μM | 3.8 | 153 | 14 | 4.1 | 426 | 5 | 1.5 | 56 | 38 | 11 |
| 5 μM | 3.7 | 46 | 46 | 4.2 | 21 | 101 | 9.2 | 20 | 106 | 9.7 |
| 7 μM | 10 | 7 | 303 | 8.4 | 17 | 125 | 3.4 | 9 | 236 | 6.6 |
| 10 μM | 5 | 2 | 1060 | 11 | 17 | 125 | 3.4 | 6 | 353 | 3.5 |

Cytotoxicity of the chemotherapeutic, paclitaxel (PTX) to P-gp overexpressing prostate cancer cells, DU145TXR, was determined in the absence and presence of the ChemGen designed 29-variants, 216, 227, 231, 541 and 551. For each experimental compound IC₅₀ values of PTX alone or in the presence of inhibitors, fold improvement of PTX sensitivity in the presence of inhibitor, and fold improvement of PTX sensitivity by variants compared to parental compound 29 are given.

At increasing concentrations, the efficacy of variant 216 in increasing paclitaxel 10 toxicity decreased when compared to the parental compound 29. At the highest concentration tested (10 μM), the 216 variant was observed to be somewhat less effective than parental compound 29 (0.4 fold compared to the 1 fold of paclitaxel+29). Unlike 216, variants 227, 231, 541 and 551 were more effective than 29 at all concentrations tested. At 3 μM, the presence of variants 227, 231, 541 and 551 resulted in 4 to 11-fold decreased paclitaxel IC₅₀ when compared to parental compound 29 and up to 38-fold overall sensitization to paclitaxel when compared to paclitaxel alone (compound 551). At higher concentrations (5 to 10 μM), addition of these variants resulted in increased paclitaxel toxicity and decreased paclitaxel IC₅₀ of up to 500-1000-fold at 10 μM, as compared to ˜100-fold sensitization caused by the parental compound 29 at 10 μM. This data indicated that variants 227, 231, 541 and 551 were better re-sensitizers of the multidrug resistant cells to paclitaxel than the original compound 29 at all concentrations tested, while variant 216 appeared to be marginally better than 29 at lower concentrations. These data demonstrate that the ChemGen generated and docking analyses selected Group 1 variants of compound 29 had increased affinity for P-gp resulting in improved efficacy for reversing chemotherapy resistance in a P-gp overexpressing cancer cell line than did the parental compound.

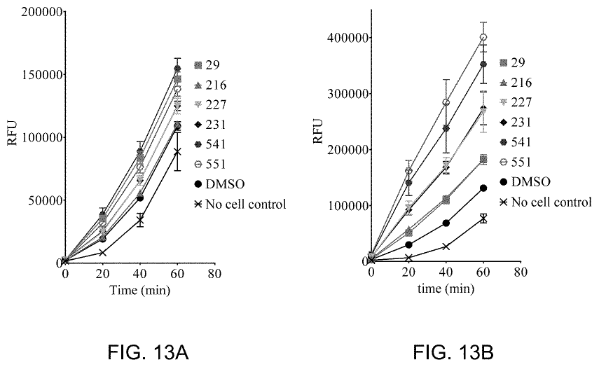

Accumulation and cellular retention of calcein AM in P-gp overexpressing prostate cancer cells upon incubation with Group 1 SMU-29 variants. Calcein AM accumulation assays have been used by us previously to evaluate P-gp-substrate accumulation in real time in the presence or absence of P-gp inhibitors²⁸. For these assays, P-gp overexpressing DU145TXR cells were incubated with the respective inhibitors in the presence of the P-gp substrate, calcein AM. Inhibition of P-gp leads to cellular accumulation of calcein AM and to cleavage of its acetoxymethyl ester groups, resulting in the generation of the highly fluorescent compound, calcein. The anionic calcein is not a substrate of P-gp and remains in the cells. In these assays, the relative fluorescence of cellular calcein was measured over time and the results of these assays are shown in FIG. 13A. The data indicated that when these cells were treated with any of the five Group 1 29 variants, the observed cellular accumulation of fluorescent calcein was lower than upon treatment with the parental compound 29. Only compound 551 resulted in marginally higher calcein accumulation than parental compound 29.

To test whether the lower accumulation of calcein in the presence of the Group 1 29 variants was the result of retention of the compounds in the cellular membrane due to their mostly increased log P values relative to 29 (Table 1), similar calcein accumulation assays were performed after a 6-hour pre-incubation with the 29-variants and parental compound 29, FIG. 13B. The inventors determined if preferential partitioning of variants in the hydrophobic part of the cell membrane may keep them more distant from the putative allosteric site on P-gp which is located adjacent to the membrane in the cytoplasm, therefore potentially slowing the inhibitory effect of the compounds. The data of FIGS. 13 and 13B show that calcein accumulation in the presence of the variants was improved by the 6-hour preincubation for all Group 1 compounds compared to the “no preincubation” experiments, supporting the idea that partitioning into the membrane may have been a contributing factor. Compound 541 which has a slightly lower log P than the parental 29 performed relatively equally to 29 without pre-incubation but exceeded 29 significantly upon the 6-hour preincubation. Compound 551 was observed to be equivalent to 29 in efficacy in these assays after the 6-hour pre-incubation.

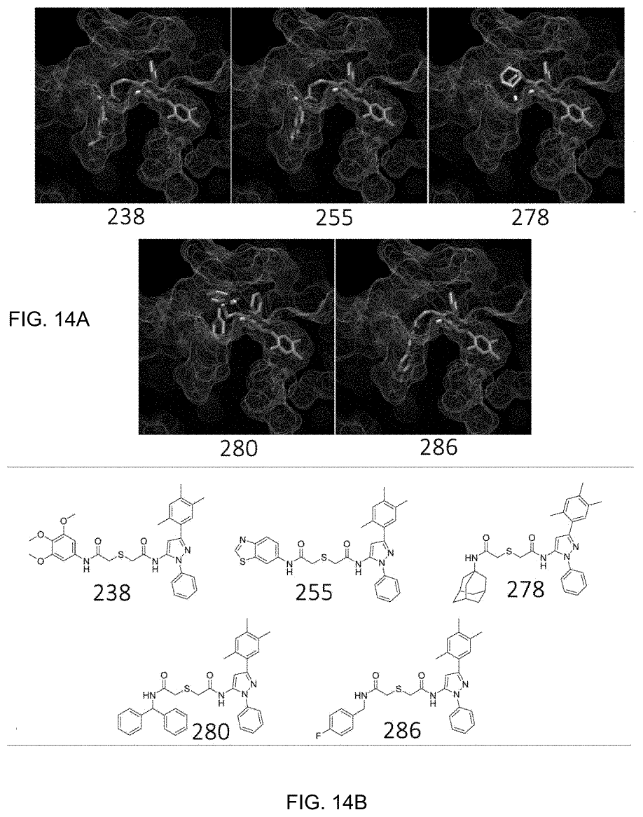

Assessing the roles of polarity and size of 29 variants in improving efficacy of compound 29 variants. To assess the contributions of overall hydrophobicity and size of the “western halves” of the 29-variants on efficacy in inhibiting P-gp and reversing multidrug resistance in cancer cells, five structural derivatives of 29 (“Group 2 variants”, FIGS. 14A and 14B) were synthesized that varied in size, shape, polar surface area, as well as overall hydrophobicity as judged by calculated values of molecular weight, topological polar surface area and log P (Table 3).

TABLE 3
29-variants differing in overall shape, size and
polarity that did not undergo docking routine.

| | | | | | Topological | |
|---|---|---|---|---|---|---|
| | | | ratio | | polar | |
| | estimated | estimated | K_{d} 29/ | Molecular | surface | |
| Synthesized | ΔG_{binding} | K_{d} | K_{d} | weight | area | Consensus |
| variant name | (kcal/mol) | (nM) | variant | (Da) | (Å²) | logP |
| 29-238 | −9.6 | 92 | 0.4 | 575 | 129.0 | 4.8 |
| 29-255 | −11.0 | 9 | 4.4 | 542 | 142.5 | 5.3 |
| 29-278 | −9.9 | 55 | 0.7 | 543 | 101.3 | 5.7 |
| 29-280 | −9.6 | 92 | 0.4 | 575 | 101.3 | 6.0 |
| 29-286 | −9.9 | 55 | 0.7 | 517 | 101.3 | 5.2 |
| ZINC08767731, | −10.1 | 40 | 1 | 461 | 108.6 | 3.8 |
| “29” | | | | | | |

Estimated K_{d} values for the ligand interactions with P-gp were calculated from the lowest estimated binding ΔG values from the AutoDock calculations. The ratio of K_{d} values is given as a relative value for potentially changed affinities exhibited by the respective variants over the parent P-gp inhibitor compound 29. Molecular weights, topological polar surface areas, and consensus log P values were calculated at the SwissADME website (http://www.swissadme.ch/) as described herein.

The variations of structure in these molecules were made in the “western” half of the molecule (FIGS. 6C-D) similar to Group 1 variants. Group 2 variants were chosen without consideration of docking results for predicted high affinity to the putative allosteric site on P-gp nor were they “counter-selected” against binding affinity to the drug binding domains of P-gp using computational docking studies. Instead, the Group 2 variants were rationally designed upon visual inspection of the putative binding site to provide a larger volume to fill the void visible beneath 29 in the putative allosteric site (FIGS. 6C-D), while at the same time decreasing the high hydrophobicity observed in the Group 1 derivatives (compare log P and TPSA values for Group1 compounds, Table 1, with those for Group 2 derivative, Table 3). Relative ease of synthesis and availability of “western” fragment precursors were also taken into account for the choice of variant synthesized. The chemical synthesis and analytic data of these Group 2 variants are given in “Supporting Information”, “Synthetic procedures” herein. Commercially available reactants and the modular synthetic approach described in FIG. 10 were used.

Table 3 shows that all five of these Group 2 variants are somewhat larger than 29, but the calculated log P values for these Group 2 compounds (Table 3) are closer to that of 29 than the log P values of the Group 1 ChemGen generated variants with the exception of 541 (Table 1). The consensus log P values^{43, 44} of 238 and 255 were calculated to be 4.8 and 5.3, while those of 278, 280 and 286 were calculated to be 5.7, 6.0 and 5.2, respectively. The topological polar surface areas (TPSA) of 238 and 255 were calculated to be higher than those of 278, 280 and 286. These TPSA values were also higher than those of the Group 1 variants, 216, 227 and 231 (compare Tables 1 and 3). Of the five structural variants in Table 3, two had increased calculated topological polar surface areas and three had reduced calculated topological polar surface areas when compared with 29.

Subsequent docking of the Group 2 variants to the putative allosteric inhibitor binding site of P-gp showed that the “western” portions of compounds 238, 255 and 286 could penetrate deeper into the hydrophobic void than does compound 29, but that compounds 278 and 280 did not seem to penetrate into the hydrophobic void as well as does compound 29 (compare FIG. 14A with FIGS. 6C and 6D).

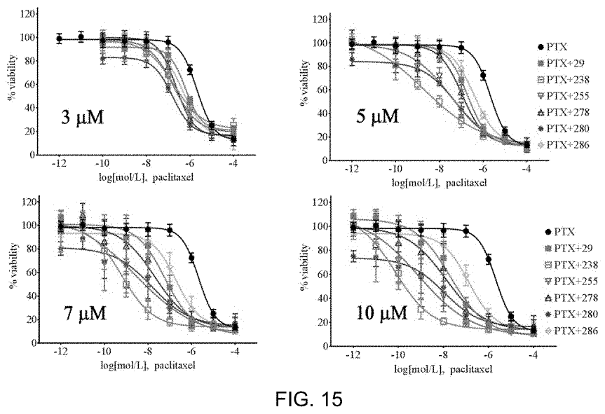

Effects of Group 2 variants on the paclitaxel sensitivity of P-glycoprotein overexpressing prostate cancer cells, DU145TXR. MTT assays were again used to assess the efficacy of the new structural 29-variants on sensitizing the chemotherapy-resistant prostate cancer cell line, DU145TXR, to paclitaxel, at concentrations of 3 μM, 5 μM, 7 μM, and 10 μM as shown in FIG. 15 and Table 4.

TABLE 4

| | | | | | Topological | |
|---|---|---|---|---|---|---|
| | | | ratio | | polar | |
| | estimated | estimated | K_{d} 29/ | Molecular | surface | |
| Synthesized | ΔG_{binding} | K_{d} | K_{d} | weight | area | Consensus |
| variant name | (kcal/mol) | (nM) | variant | (Da) | (Å²) | logP |
| 29-238 | −9.6 | 92 | 0.4 | 575 | 129.0 | 4.8 |
| 29-255 | −11.0 | 9 | 4.4 | 542 | 142.5 | 5.3 |
| 29-278 | −9.9 | 55 | 0.7 | 543 | 101.3 | 5.7 |
| 29-280 | −9.6 | 92 | 0.4 | 575 | 101.3 | 6.0 |
| 29-286 | −9.9 | 55 | 0.7 | 517 | 101.3 | 5.2 |
| ZINC08767731, | −10.1 | 40 | 1 | 461 | 108.6 | 3.8 |
| “29” | | | | | | |

The data suggest that the presence of all of the variants except for 286 increased the paclitaxel toxicities to these P-gp overexpressing cells. Compounds 238 and 255 increased paclitaxel toxicities to the greatest extents: At 5 μM concentration, compound 238 decreased the paclitaxel IC₅₀ by about a thousand-fold, similar to compound 255 at 7 μM. Comparison with parental compound 29 showed that 238 improved sensitization of DU145TXR to paclitaxel by 3.4-fold at 3 μM, and by around 100-fold at 5 and 7 μM. At 10 μM, compound 238 resulted in a 300-fold decreased paclitaxel IC₅₀ of DU145TXR compared to parental compound 29 at the same concentration. Compound 255 also showed substantial improvement in sensitization of DU145TXR to paclitaxel that was comparable to 238 at 3 μM, but the effect was not as pronounced at higher concentrations. The other three compounds, 278, 280 and 286, were similar or less effective than the Group 1 variants, 216, 227 and 231. In addition, compound 280 seemed to exhibit some toxicity to the multidrug resistant cancer cells as judged by the lowered cell viability at very low concentrations of paclitaxel in the presence of 280 (FIG. 15).

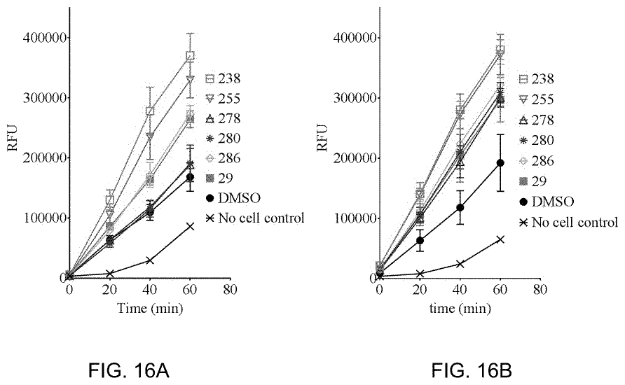

Accumulation and cellular retention of calcein AM in DU145TXR in the presence of 29-variants from Group 2. FIG. 16A shows that without pre-incubation, the presence of 5 μM of compounds 238 and 255 resulted in considerably increased calcein accumulation in DU145TXR cells when compared to 29. The effect of compound 286 was comparable to 29, while 278 and 280 caused less calcein accumulation than the parental compound. After a 6-hour pre-incubation (see FIG. 16B), the effectiveness of inhibiting P-gp catalyzed transport of calcein AM remained strongest for the more polar 29 variants 238 and 255, while that of the more hydrophobic variants 278, 280 and 286 was comparable to 29. Even though variant 286 has a consensus log P value similar to the log P calculated for 238 and 255, it seems to group better with regard to efficacy in blocking calcein AM export with compounds that have comparable or lower calculated polar surface areas, i.e. 278 and 280, see Table 3, and 216, 227, and 231 from Group 1, Table 1. Group 1 compounds 216, 227 and 231, in addition, have increased consensus log P values which might explain their somewhat reduced efficacy in blocking P-gp catalyzed calcein AM export (Table 1 and FIG. 13).

Evaluation of mode of inhibition of P-glycoprotein by Group 1 and Group 2 variants of compound 29. To assess the mode of inhibition of P-gp by the novel variants of P-gp inhibitor 29, ATP hydrolysis by P-gp was evaluated in the presence or absence of the variants. Both “basal” ATP hydrolysis (assayed in the absence of added transport substrate) and “stimulated” ATP hydrolysis (assayed in the presence of the P-gp transport substrate, verapamil) were assessed as described in reference 2⁶⁶. Murine P-gp (MDR3) expressed in Pichia pastoris that had all naturally occurring cysteines replaced with alanine, was used^{45, 46}. It is widely assumed that the ATP hydrolytic rate of P-gp is stimulated in the presence of transport substrates when compared to ATP hydrolysis in the absence of transport substrates. Assays comparing these rates can therefore be useful not only in identifying inhibitors of P-gp catalyzed ATP hydrolysis, but also to potentially infer whether a compound might be a transport substrate if basal ATPase rates are stimulated by the addition of a compound.

Effects of compound 29 variants on verapamil-stimulated ATP hydrolysis by P-gp. The effects of compound 29 variants on P-gp ATP hydrolysis rates assayed in the presence of verapamil (a good substrate for transport by P-gp) are presented in Table 5 A (“Stimulated ATPase”).

TABLE 5
Mode of Inhibition of Cysteineless Mouse MDR3 P-glycoprotein
by Group 1 and Group 2 compound 29 variants.

| | | | | | | | Cellular |
|---|---|---|---|---|---|---|---|
| | | | | | | | accumulation:ratio |
| | Stimulated | | | | | | of plus |
| | ATPase | | Effect on | Basal ATPase | | Effect on | Tariquidar |
| | (% of DMSO \| | | stimulated | (% of DMSO \| | | basal | over no |
| Compound | significance) | | ATPase | significance) | | ATPase | Tariquidar |
| DMSO | 100 ± 8 | — | — | 100 ± 7 | — | — | — |
| SMU29 | 49 ± 2 | ** | inhibitor | 95 ± 11 | NS | none | 1.0 |

Group 1 - ChemGen and docking selected

| | | | | | | | |
|---|---|---|---|---|---|---|---|
| SMU29-216 | 108 ± 2 | NS | none | 105 ± 7 | NS | none | 1.1 |
| SMU29-227 | 50 ± 2 | ** | inhibitor | 88 ± 4 | NS | none | 1.0 |
| SMU26-231 | 141 ± 18 | * | stimulator | 70 ± 0 | * | inhibitor | 0.9 |
| SMU29-541 | 68 ± 7 | * | inhibitor | 101 ± 12 | NS | none | 1.0 |
| SMU29-551 | 62 ± 5 | ** | inhibitor | 150 ± 6 | ** | stimulator | 1.1 |

Group 2 - Rationally designed/no docking selection

| | | | | | | | |
|---|---|---|---|---|---|---|---|
| SMU29-238 | 194 ± 22 | ** | stimulator | 1143 ± 46 | ** | stimulator | 1.9 |
| SMU29-255 | 123 ± 15 | NS | none | 355 ± 7 | *** | stimulator | 1.2 |
| SMU29-278 | 41 ± 7 | ** | inhibitor | 78 ± 4 | * | inhibitor | 1.0 |
| SMU29-280 | 116 ± 4 | * | stimulator | 148 ± 4 | * | stimulator | 1.2 |
| SMU29-286 | 98 ± 2 | NS | none | 143 ± 32 | NS | none | 1.3 |

| | Cellular | | | SL-ANP | |
|---|---|---|---|---|---|
| | accumulation:ratio | | Maximum | binding to | |
| | of plus | | ATP binding | P-gp | |
| | Tariquidar | Transport | (mol SL-ANP | Apparant | Effect on |
| | over no | substrate | bound/mol | Kd | SL-ANP |
| Compound | Tariquidar | for P-gp | P-gp) | (μM) | binding |
| DMSO | | — | 1.8 ± 0.1 | 36.5 ± 3.6 | — |
| SMU29 | NS | no | [symbol] | 71.0 ± 12.6 | no |
| Group 1 - ChemGen and docking selected | | | | | |
| SMU29-216 | NS | no | 1.7 ± 0.1 | 23.1 ± 3.9 | no |
| SMU29-227 | NS | no | 1.8 ± 0.1 | 36.9 ± 4.1 | no |
| SMU26-231 | NS | no | 1.8 ± 0.1 | 22.2 ± 3.8 | marginally |
| SMU29-541 | NS | no | 1.8 ± 0.1 | 24.1 ± 4.0 | no |
| SMU29-551 | * | no | 1.7 ± 0.1 | 22.8 ± 3.6 | no |
| Group 2 - Rationally designed/no docking selection | | | | | |
| SMU29-238 | *** | yes | 1.2 ± 0.1 | 20.1 ± 4.0 | yes |
| SMU29-255 | * | yes | 1.9 ± 0.1 | 25.6 ± 4.7 | no |
| SMU29-278 | NS | no | 1.3 ± 0.1 | 22.9 ± 4.5 | yes |
| SMU29-280 | NS | yes | 1.6 ± 0.1 | 21.6 ± 4.6 | marginally |
| SMU29-286 | * | yes | 1.9 ± 0.1 | 25.7 ± 4.9 | no |

[symbol]

indicates data missing or illegible when filed

ATP hydrolysis assays using purified P-glycoprotein were performed without added transport substrate (“basal ATPase”) or in the presence of verapamil (“Stimulated ATPase”). Results are presented compared to DMSO control standard deviation (three independent experiments with duplicate samples). Basal activity of P-gp was 20 to 30 nmol/min mg, verapamil-stimulated rates were 200 to 400 nmol/min mg P-gp. Stimulation of basal ATPase by 29-variants was used as an indicator that a compound may be a P-gp transport substrate. Effects on stimulated P-gp ATPase activity indicated whether a compound directly interfered with ATP usage by the protein (***, p<0.001; **, p<0.01; *, p<0.1; NS, not significant). Quantitative cellular accumulation of 29-variants was performed using LC-MS/MS and is presented as a ratio of the cellular amounts of 29-variants in the presence of P-gp inhibitor, tariquidar, divided by amounts accumulated in its absence. A ratio >1 indicates that the compound likely is a transport substrate of P-gp (***, very significant; *, significant; NS, not significant). Binding of an ATP analog, SL-ATP, to P-gp was used to determine whether ATP binding to P-gp was affected by the 29-variants. Values +/−standard deviations are shown for at least three different P-gp preparations and three independent SL-ATP titration experiments. The values for SL-ATP binding in the presence of 29 were taken directly from Brewer et al. (2014).

The respective percent ATPase activity is shown, normalized to the ATPase in the presence of DMSO carrier/no added experimental compound. Interestingly, the Group 1 compounds differed in their effects on “stimulated” ATPase: 216 did not affect stimulated ATP hydrolysis activities, while compounds 227, 541 and 551 inhibited activity similar to parental compound 29. Compound 231 slightly stimulated ATP hydrolysis rates in the presence of verapamil. For Group 2 compounds, 238 stimulated the “stimulated” ATPase rates by about two-fold, while variant 280 showed only a slight stimulation of hydrolysis rates and compounds 255 and 286 had no significant effect. Only compound 278 of the Group 2 variants inhibited verapamil-stimulated ATP hydrolysis by P-gp similar to the parental compound 29.

Effects on “basal” ATP hydrolysis rates of compound 29 variants. Group 1 compounds 216, 227 and 541 did not significantly affect basal ATP hydrolysis by P-gp, while compound 231 inhibited the basal ATPase rates of P-gp. Only 551 of the Group 1 molecules stimulated basal ATPase activities of P-gp. Of the Group 2 compounds, 238, 255 stimulated basal ATPase by ˜10 and ˜3 fold respectively. Compounds 280 and 286 stimulated basal ATPase only marginally or with no statistical significance. Only compound 278 inhibited basal ATPase of P-gp. The relatively strong activation of basal ATPase by compounds 238 and 255 was suggestive that these two compounds and potentially to a lesser extent, compound 280, may be transport substrates of the pump. Compound 278 was not indicated to be a good transport substrate for P-gp since it inhibited basal ATPase by P-gp.

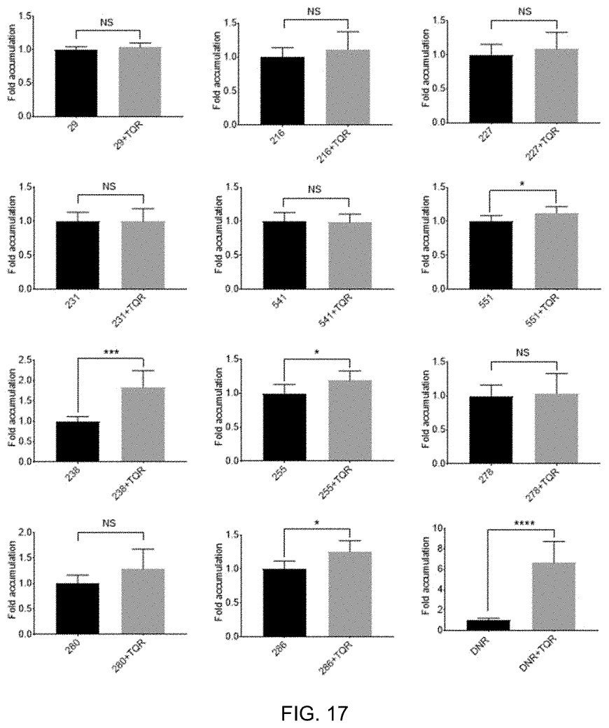

Intracellular accumulation of compound 29 variants. Cell accumulation assays for each of the 29 variants were performed as in reference²⁸ to more directly assess whether the compounds were indeed transport substrates for P-gp. These assays measured the intracellular accumulation of the experimental compounds using LC-MS/MS methods after incubation with the P-gp over-expressing cell line, DU145TXR, in the absence and presence of the strong P-gp inhibitor, tariquidar⁴⁷ (TQR). Low levels of cellular accumulation of a compound in the absence of tariquidar accompanied by much higher levels of accumulation in the presence of tariquidar suggests that the compound in question may be a transport substrate of P-gp. In other words, if a compound is an effective transport substrate for P-gp, active P-glycoprotein in these cells would limit intracellular accumulation, while inhibited P-gp would result in higher intracellular concentrations. Daunorubicin (DNR) is an example of a good transport substrate for P-gp and showed very strong cellular accumulation in these assays when P-gp was inhibited by tariquidar, but much less accumulation in the cells when P-gp was not inhibited (see FIG. 17, DNR). If a compound is not a substrate of P-gp, no significant difference in intracellular accumulation of the compound with or without tariquidar is expected. FIG. 17 “29”, shows that compound 29 is not a transport substrate for P-gp²⁸ and that no significant difference in cellular accumulation of 29 was observed with or without addition of tariquidar (“TQR”). FIG. 17 also presents the fold accumulation of each of the experimental 29-variants in these assays normalized to the amount of compound found in the absence of TQR. This data is numerically presented in Table 5 as the ratio of observed accumulation in the presence of tariquidar divided by the accumulation observed in the absence of tariquidar for each of the experimental compounds. Ratios that are significantly greater than 1.0 indicate that a compound is very likely a transport substrate of P-gp.

None of the Group 1 molecules tested resulted in intracellular accumulations that were considerably different in the absence versus presence of TQR, similar to the parental compound 29 (FIG. 17 and Table 6), indicating that the variants were not transport substrates of the pump in human cells in culture. This data somewhat correlates with the observation that 216, 227 and 541 did not activate basal ATPase activities by P-gp. Compound 231 also showed no significant cellular accumulation in the presence of TQR but marginally activated basal ATPase activity of P-gp. Taken together, these results suggest that none of the Group 1 compounds are good transport substrates for P-gp. However, compound 551 stimulated basal ATPase activity while accumulation assays strongly suggested that the variant was not a pump substrate, suggesting that the correlation between stimulation of basal ATPase activity and transport substrate may not be as clear-cut as originally thought.

Of the Group 2 compounds, variant 238 showed a very large and significant increase in intracellular accumulation in the presence of TQR (FIG. 17 and Table 6). Compounds 255 and 286 showed more modest, but statistically significant increases in intracellular accumulation when P-gp was inhibited in the presence of TQR. Compounds 278 and 280 did not show significantly different intracellular accumulations in the absence or presence of TQR. Based on the activation of basal ATPase activities by 238, 255 and 286 and supported by their cellular accumulation data, these three Group 2 variants of 29 are very likely to be transport substrates of P-gp. Compound 280, based on its activation of basal ATP hydrolysis, may also be a transport substrate of P-gp, but is not likely to be a good substrate. Group 2 compound 278 is very unlikely to be a transport substrate of P-gp, since it neither activates basal ATPase nor did it show significantly increased cellular accumulation in the presence of tariquidar.

To assess whether the observed discrepancies of compounds stimulating basal P-gp ATPase activity but not being transport substrates of the human pump in the cell culture assessments were due to the fact that these biochemical assays used a cysteineless variant of the mouse MDR3 P-glycoprotein, the experiments were repeated using normal human MDR1 P-gp. In order to stabilize the human protein for the activity assays, the protein was assembled into membrane nanodiscs as described herein. The results of the experiments are shown in Table 6.

TABLE 6
Effects of Group 1 and Group 2 compound 29 variants on
ATP Hydrolysis by Normal Human MDR1 P-glycoprotein.

| | Stimulated ATPase | | Effect on | Basal ATPase | | Effect on |
|---|---|---|---|---|---|---|
| | (% of DMSO \| | | stimulated | (% of DMSO \| | | basal |
| Compound | significance) | | ATPase | significance) | | ATPase |
| DMSO | 100 ± 6 | — | — | 100 ± 5 | — | — |
| SMU29 | 62 ± 3 | ** | inhibitor | 88 ± 9 | NS | none |

Group 1 - ChemGen and docking selected

| | | | | | | |
|---|---|---|---|---|---|---|
| SMU29-216 | 64 ± 6 | ** | inhibitor | 69 ± 8 | ** | inhibitor |
| SMU29-227 | 62 ± 5 | ** | inhibitor | 83 ± 7 | NS | none |
| SMU29-231 | 40 ± 3 | **** | inhibitor | 70 ± 4 | *** | inhibitor |
| SMU29-541 | 66 ± 6 | ** | inhibitor | 87 ± 9 | NS | none |
| SMU29-551 | 27 ± 1 | *** | inhibitor | 72 ± 9 | * | inhibitor |

Group 2 - Rationally designed/no docking selection

| | | | | | | |
|---|---|---|---|---|---|---|
| SMU29-238 | 61 ± 7 | ** | inhibitor | 89 ± 7 | NS | none |
| SMU29-255 | 59 ± 6 | ** | inhibitor | 84 ± 9 | NS | none |
| SMU29-278 | 63 ± 6 | ** | inhibitor | 74 ± 6 | ** | inhibitor |
| SMU29-280 | 54 ± 7 | *** | inhibitor | 76 ± 8 | * | inhibitor |
| SMU29-286 | 59 ± 4 | *** | inhibitor | 95 ± 3 | NS | none |

ATP hydrolysis assays using purified P-glycoprotein were performed without added transport substrate (“basal ATPase”) or in the presence of verapamil (“Stimulated ATPase”). Results are presented compared to DMSO control ±standard deviation (three independent experiments with duplicate samples). The specific basal activity of normal human MDR1 P-gp was between 123 and 193 nmol min⁻¹g⁻¹, and transport substrate (verapamil) stimulated activity was between 193-263 nmol min⁻¹mg⁻¹. Effects on stimulated P-gp ATPase activity indicated whether a compound directly interfered with ATP usage by the protein (***, p<0.001; **, p<0.01; *, p<0.1; NS, not significant).

Interestingly, neither compound 29 nor any of is variants had a stimulatory effect on basal ATPase activity of the normal human protein reconstituted into membrane nanodiscs, while all of them significantly inhibited transport substrate (verapamil) stimulated activity. The results clearly indicate that the source (human vs. mouse) and potentially also the membrane environment of P-glycoprotein strongly affects the overall behavior of potential biochemical inhibitors.

Effects of 29 variants on binding of an ATP-analog to purified P-glycoprotein. ATP binding in the presence of the 29 variants was assessed in titration assays using a spin-labeled analog of ATP, 2′,3′-SL-ATP (2′,3′-(2,2,5,5,-tetramethyl-3-pyrroline-1-oxyl-3-carboxylic acid ester) ATP; (2′,3′ indicates a rapid equilibrium of the ester bond between the C2′ and C3′ of the ribose moiety))⁴⁸⁻⁵⁰, and electron spin resonance spectroscopy as described in²⁶. Due to the lower stability of the human P-glycoprotein in the extended times needed for these experiments, the cysteineless mouse protein was used here. The goal was to assess whether binding of the 29 variants to P-gp affected nucleotide binding to the protein. Results of these assays are presented in Table 5A. Except for compounds 238 and 278, neither of which initially underwent the selective docking routines used for Group 1 compounds, none of the novel inhibitors affected maximal binding of the ATP analog or the apparent K_{d}, showing that the inhibitors were indeed targeted to the putative allosteric binding site on P-gp. Compounds 238 and 278 reduced SL-ATP binding to about 1 mol SL-ANP (adenine nucleotide with an undefined number of phosphoryl groups) bound per mol of enzyme, suggesting that these inhibitors may also interact with the nucleotide binding sites or may indirectly induce changes in nucleotide binding to P-gp that affect ATP binding.

In addition to evaluating the effects of the decreased hydrophobicity of the Group 2 variants on the reversal of MDR in cell-based assays, it was also of interest to evaluate whether or not the docking routines employed for the Group 1—ChemGen derived variants of 29 were better able to predict compounds that were not transport substrates of P-gp when compared to the Group 2 compounds. All five of the Group 1 molecules that were chosen though the subtractive docking routine and that were predicted to not interact well with the drug binding domains were observed to not be transport substrates. When Group 2 molecules were docked to a structural model of P-gp that was essentially identical to the one initially used to identify parental compound 29 as a P-gp inhibitor²⁶ as well as to choose Group 1 variants, four of the five molecules were predicted to interact well with the drug binding domains and one was not predicted to bind well (data not shown). Three of these five predictions were confirmed by the LC MS/MS experiments described above. All in all, the experimental data for Groups 1 and 2 suggested that the subtractive docking method was predicting the correct outcome (transport substrate vs. no transport substrate) in 4 out of 5 cases or at 80%.

While optimization efforts of hit compound 29 did not include aspects of compound toxicity, it seems of interest to note that only one of the 29-variants (compound 541) showed some toxicity in cell viability assays in the P-gp overexpressing DU45TXR¹³ cancer cells when administered in the absence of chemotherapeutic. No significant toxicity of the compounds was observed in non-cancerous human lung fibroblast cells, HFL-1⁵¹ (data not shown). In addition, toxicity of the chemotherapeutic, paclitaxel, was not increased in the presence of 29 or 29-variants in cells that do not overexpress P-gp, i.e. HFL-1 and the not chemotherapy resistant, not P-gp overexpressing prostate cancer line, DU145⁵² (data not shown). The overall results indicate that increased lethality of paclitaxel to the P-gp overexpressing cells was due to the inhibition of the pump and increased accumulation of paclitaxel to therapeutic levels within the cells. It should be noted that compounds 541 and 551 were not assessed in these latter experiments.

Using computational approaches to create novel variants of “hit” molecules from drug discovery programs. In drug development, often large sets of molecules that are related to a molecule of interest are synthesized to identify derivative molecules with better drug-like characteristics than those of the originally identified molecule. The required organic chemistry syntheses are expensive, costly in time, can be very laborious, and require the skills and time of highly qualified chemists. Often hundreds or thousands of compounds are synthesized with only a few variants showing desired improvements in pharmacological characteristics. Even in these early steps of a medicinal chemistry project, these efforts add to the already significant costs of drug development⁵³. While some drug-like characteristics can be estimated from computational approaches, most require biochemical, biophysical, pharmacological, cell biological and/or animal experimentation to assess potential improvement over the parental compound, which again increases the time and expenditures required for each potential lead compound.

A number of virtual chemical synthesis computer programs have been previously described. Some use fragments annotated with reaction rules²⁹ or compound scaffolds with chemically reactive linkers³⁰, and still others use popular click chemistries that can easily translate into the laboratory^{31, 32} just to mention only a few. To make more informed choices about which of the vast numbers of possible compound variants to synthesize for subsequent testing, a set of computational routines (collectively called ChemGen) have been written and developed to synthesize in silico what can be very large numbers of variant compounds. The methods differ from predecessor methods in that retrosynthetic approaches to the discovered hit molecule synthetic routes are mimicked in the computations. This results in advantageous translation to actual chemical syntheses of identified variants of interest, is not constrained to one or a few chemical reaction types, but can theoretically encompass any chemical reaction. A disadvantage is that each reaction type must be programmed ahead of its implementation, but the ChemGen platform may be adapted to new chemistries relatively easily.

Next, the inventors determined whether an increase in the efficiency of drug development can be achieved by performing iterative virtual molecular syntheses using efficient computational approaches instead of physically synthesizing a large set of compounds related to a molecule of interest. After production of the virtual compound variant library, the new molecules were computationally assessed for predicted improvements in any pharmacological characteristics that can be calculated, including relatively simple physiochemical data (topological polar surface area or TPSA, log P values, molecular weight, etc.) as well as more complex indicators of improved drug-like characteristics such as predicted toxicities, mutagenicity, likelihood of inducing potential drug-drug interactions (cytochrome P450 isozyme substrate character, etc.). Other important factors that can be calculated and that are valuable for decision making are predicted increased binding affinities to targeted proteins as well as potentially decreased binding affinities to undesired protein targets of the drug lead compounds. In recent years, machine learning machine methods have been employed for predicting toxicities, various ADME characteristics and even protein-ligand binding affinities of molecules of potential interest⁵⁴⁻⁵⁹.

By creating hit variants computationally and then assaying them—again computationally—for improved characteristics, variants that do not possess the desired improved characteristics can be eliminated from consideration before any actual organic synthetic chemistry is performed. This path can lead to expedited and much more cost-effective syntheses of a relatively small number of potentially improved hit-variants. The latter part of this approach, namely computational counter-selection against compounds with characteristics that are undesirable, was used by us previously to identify molecules that inhibited P-gp catalysis, but that were not transport substrates of P-gp²⁶⁻²⁸. Counter selections such as these, used to eliminate from consideration compounds with undesirable target interactions, can be extended to any property of a molecule that is calculable. When coupled with virtual synthesis of hit variants, increasing the efficiency and cost-effectiveness of synthesis programs is practically assured.

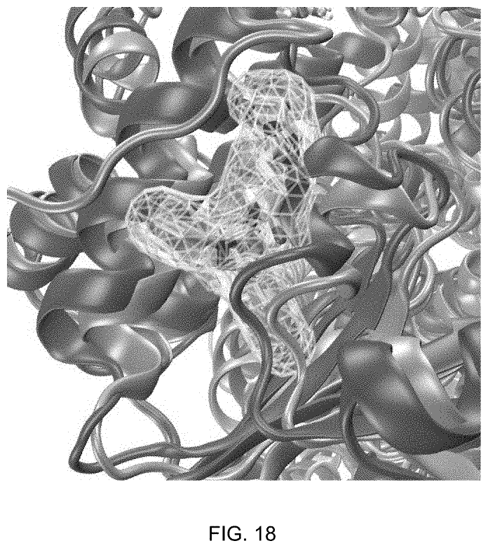

Ligand docking methods as described in²⁶ have previously led us to identify the P-glycoprotein inhibitor, compound 29, that served as the initial hit for further drug development. Compound 29 and several other hits discovered were evaluated in biochemical and biophysical studies for their mechanism of inhibition of P-gp action²⁶, as well as for their potential to reverse multidrug resistance in different cancer cell lines in culture^{27, 28} Compound 29 was chosen here as an initial compound for further development mostly for the fact that binding of compound 29 was predicted to be at an allosteric site, away from the nucleotide binding sites of P-gp²⁶. Biophysical assessment using electron spin resonance spectroscopy and a spin-labeled ATP analog suggested that ATP binding was not affected in the presence of the inhibitor, while ATP hydrolysis assays showed inhibition of ATPase activity²⁶. A putative mechanism for P-gp inhibition by 29 can be envisioned when comparing the position to which 29 docked with high affinity²⁶ in FIG. 1B to the recently published cryo-EM structure of the protein¹⁶ (FIG. 18). FIG. 18 shows a pronounced steric clash of P-gp amino acid side chains with the bound inhibitor when P-gp adopts a conformation similar to that of the published cryo-EM structure. This clash may indicate that P-gp cannot undergo conformational changes that may be needed for catalytic activity when a small molecule is occupying this putative allosteric binding site. Creating variants of compound 29 with increased affinity to this particular binding site was therefore viewed as a promising strategy towards the further development of a specific P-gp inhibitor that would not function as a transport substrate of the protein.

A closer evaluation of the high affinity allosteric docking site of compound 29 to P-gp revealed a relatively large hydrophobic pocket where the cyclopropyl moiety of the “western” half of the molecule interacted with the protein (FIGS. 1A, C, and D). In the effort to assess putative P-gp inhibitors with increased affinity to the protein, the goal was to “virtually synthesize” a number of 29 variants with larger moieties at the “western” half of the protein. The synthesis scheme shown in FIG. 2B and the ChemGen protocols described herein were used to accomplish this goal. The resulting 647 derivatives of hit compound 29 were evaluated for binding to the allosteric site and counter-screened for low affinity interactions to the drug binding domains of the protein using docking methods that were similar to the screens described in²⁶ and that led to the discovery of the parental compound 29. This subtractive screening protocol has been shown to be effective in predicting inhibitors of P-gp that are not transport substrates²⁸. The “Group 1—ChemGen synthesized and docking selected” 29 variants were ranked by binding affinity to the allosteric site on P-gp. Compounds with molecular weights that exceeded 627 Da were excluded from the further evaluations. The remaining 12 variants of 29 that were predicted to bind with relatively high affinity the proposed allosteric site were then evaluated for some physicochemical characteristics (Table 1). All of the variants showed higher molecular weights as was assumed due to the larger fragments being added to the “western” part of the molecule. TPSA and consensus log P values differed between the variants. Visual evaluation of the docking poses of the variants showed clear overlap of the “eastern” parts of the molecules, highlighting the consistency of docking to this site on P-gp. The “western” portions of the Group 1 29 variants were observed to extend into the hydrophobic pocket of the protein that was observed to reach beyond the cyclopropyl group of the original compound 29 (FIG. 11 and FIGS. 6C-D). It was this pocket in P-gp that it was undertaken to better fill using the ChemGen produced variant compounds.

Five of the 29 variants from Table 1 (216, 227, 231, 541 and 551) were chosen for actual chemical synthesis mostly based on the perceived ease of synthesis and expense of precursor fragments. All three variants added more volume to the “western” half of the molecules and all but 541 had lower TPSA and higher log P values than the original compound 29. Closer inspection of the docking poses of the three variants (FIG. 11) revealed that 29 derivatives 216 and 231 both reach significantly farther into the hydrophobic pocket than do either the parental compound 29, while variant 227 seems to make significant protein interactions at the “mouth” of the hydrophobic pocket. Both 541 and 551 reached deeply into the previously observed pocket within P-gp. Using the novel method of the present invention, the inventors found that these five variants should show improvements in reversing the MDR phenotype of cancer cells that overexpress P-gp as demonstrated for 29 in^{27, 28} and would therefore be reasonable initial choices.

Cell viability assays using a P-gp overexpressing prostate cancer cell line indicated that all five of the 29 variants had improved characteristics over the parental 29 for causing cell mortality (summarized in Table 2). This remarkable result, that five out of five Group 1—ChemGen produced variants showed between 2.4-fold and 11-fold improvement over the performance of 29 in reversing P-glycoprotein-conveyed multidrug resistance phenotype (summarized in Table 2), underscores the utility of the ChemGen virtual synthesis approach and the employed docking selections for increasing affinity to P-gp. It is a reasonable conclusion that the larger Group 1 variants were able to interact more strongly with the protein as depicted in FIGS. 1 and 3 than did the parental compound 29 (Table 1, FIGS. 9A and B, FIG. 11).

In assays designed to allow quantification of the accumulation of the P-gp transport substrate, calcein AM, in cells that over-express P-gp, however, the larger, more hydrophobic Group 1 variants did not perform better than 29. Comparison of the calculated consensus log P values for variants 216, 227, 231 and 551 (6.6, 6.4, 5.7, and 5.9 respectively, Table 1) with the log P of the parental compound 29 (4.0, Table 1) led us to ask whether the lack of efficacy in inhibiting P-gp-catalyzed export of calcein AM may have been due to 29 variants being too hydrophobic to efficiently transfer across the cellular membrane to the cytosol-located nucleotide binding domains of P-gp for efficacious inhibition in the nucleotide binding domain of the protein to occur. The relatively short incubation times used in the calcein AM assays as shown in FIG. 5A could exacerbate this problem when compared to much longer exposure times in the cellular toxicity assays (FIG. 4). Supporting this view were the observations of slight improvements in efficacy in the calcein AM assays when a longer pre-incubation with the variants was performed, but none of the variants performed better than 29 in this assay. These latter results supported the inference that increased hydrophobicity of the variants decreased their efficacy in these P-gp transport substrate accumulation assays. Interestingly, the 29-variant 541 which has a consensus log P similar to 29 performed similar to the parental compound even without preincubation but then exceeded 29 upon preincubation. This may indicate that while polarity of the compound is important for cell entry the “fit” of a variant into a protein pocket enhances efficacy.

Since the ChemGen/docking routine computational approach for selecting variants at the putative allosteric site resulted in larger but mostly also more hydrophobic “western” fragments in variants 216, 227, 231, 541 and 551 and since increased hydrophobicity may have limited utility of the compounds, the goal was to rationally design other variants of the “western” portion of compound 29 that might result in improved interactions with P-gp as well as more favorable physicochemical characteristics. In this light, five additional “western” derivatives of 29 were synthesized with varying shape, size and physicochemical properties, compounds 238, 255, 278, 280 and 286 (Group 2 variants). Again, the compounds were chosen for relative ease of synthesis as well as availability and expense of precursors. Unlike the previous set of ChemGen generated 29 variants (Group 1 variants), this latter set did not initially undergo the docking routines that selected for high affinity binding to the nucleotide binding domains and low affinity to the drug binding domains that were used to identify the parental compound 29 and the Group 1 variants. Instead these variants were chosen by visual inspection of the putative binding site, as well as by the calculated physicochemical properties of the resulting variants.

Efficacy of rationally designed Group 2 variants in reversing MDR caused by P-gp over-expression. If compounds 216, 227, 231 and 551 were too hydrophobic for favorable passage through the cellular membrane, thereby causing lowered efficacy of P-gp inhibition and decreased calcein AM accumulation in the P-gp overexpressing cells, then the more hydrophilic Group 2 derivatives, 238 and 255, as judged by their TPSA values and possibly 286 as judged by its log P value, might show increased re-sensitization of MDR cells to paclitaxel as well as improved calcein AM retention as compared to the other derivatives. These predictions were also based on the observed docking poses shown in FIG. 14A where one can see that 238, 255 and 286 fill the targeted void in P-gp much better than do parental compound 29 or Group 2 variants 278 and 280. Interestingly, compounds 238 and 255 vastly outperformed compounds 278, 280 and 286 in reversing MDR caused by P-gp over-expression (summarized in Table 4), which once again underlines the complexity of these interactions. In these cases TPSA values greater than ˜110 Å³ and consensus log P values <˜6, correlated best with MDR reversal efficacy in both the Group1 and Group 2 variants.

Effectiveness of the selective docking routines used on Group 1 variants to ensure targeting to the nucleotide binding domains and avoid the drug transport domains of P-gp. It was also of interest to investigate whether the subtractive docking routine performed on Group 1 variants added value to the overall process in selecting variants for synthesis and subsequent testing. Specifically, the docking selections employed were aimed at identifying compound 29 variants that preferentially bound to the nucleotide binding domains and were therefore not good transport substrates of P-gp. Comparison of the results from Group 1 compounds to the more traditional rationally designed Group 2 variants that were chosen without additional input on docking derived binding affinities to different substructures of the protein was therefore made. As can be seen in Table 5A, only compound 551 of the Group 1 variants selected through the ChemGen/docking routines stimulated “basal” ATP hydrolysis rates, and compound 231 inhibited basal ATPase activities. Intracellular accumulation assays which more directly measure whether a compound is a transport substrate of P-gp (see reference²⁸) confirmed the predictions (Table 5, FIG. 17). The lack of statistical difference in the results of the intracellular accumulation assays in the presence and absence of the strong P-gp inhibitor tariquidar (Table 5, FIG. 17) for all five Group 1 compounds (compounds 216, 227, 231, 541 and 551) strongly suggests that none of the Group 1 compounds functions as a transport substrate of P-gp. To evaluate whether the stimulatory effect on basal ATPase activity of 551 may have been due to the fact that these biochemical assays were performed using a cysteine-less variant of the mouse MDR3 P-glycoprotein and not the human isoform, the experiments were repeated using normal (not cysteine-less) human P-gp that had been reconstituted into nanodiscs for enhanced stability. The results presented in Table 6 indicate that none of the 29 variants, nor 29 itself, stimulated basal ATP hydrolysis but that, in contrast, several of them inhibited basal ATPase activity.

On the other hand, intracellular accumulation data (summarized in Table 5) and stimulation of “basal” ATP hydrolysis activity of the mouse cysteine-less P-gp that was induced by Group 2 compounds 238, 255 and 286 (also summarized in Table 5) correlated quite well. Although the intracellular accumulation for compound 280 with or without addition of tariquidar were not observed to be significantly different, compound 280 did stimulate “basal” ATP hydrolysis in the absence of any other added transport substrate, which may indicate that this compound may also be a transport substrate of P-gp. However, the lack of stimulation of the human P-gp lets this conclusion be more doubtful. Compound 278 of Group 2 did not show differences in intracellular accumulation with or without tariquidar, but showed strong inhibition of both “basal” and verapamil-stimulated ATP hydrolysis of both orthologous enzymes. Compound 278 is therefore the only member of Group 2 that one can conclude is definitely not a transport substrate of P-gp, while 4 of the 5 Group 2 compounds either were transport substrates of P-gp or were potentially transport substrates of P-gp. In contrast to these conclusions, none of the five Group 1 molecules were transported by P-gp. Although the general case cannot be statistically proven since the number of synthesized and tested variants was too small, these studies show that the ChemGen produced variants that were selected against interactions at drug transporting structures on P-gp were much more likely not to be transport substrates of P-gp (five of five Group 1 variants were not observed to be transport substrates) than the Group 2 molecules that did not undergo the docking counter-selection procedures (where 4 out of 5 molecules were likely or potentially transport substrates for P-gp). It is very clear therefore that the docking and counter-selections were very effective in identifying 29 variants that were not transported by P-gp and that these procedures out-performed the rationally designed molecules that were not subjected to these selections.

Mechanism of action of the 29 variants. The effects of the 29 variants on verapamil-stimulated ATP hydrolysis rates catalyzed by P-gp (Table 5A and B) are consistent with the effects observed for the compounds in reversing MDR caused by P-gp (Tables 2 and 4). Group 1 compound 216 and Group 2 compound 286 had little effect in either the stimulated ATPase or the MDR reversal assays, likely indicating that these compounds do not strongly interact with P-gp under the conditions tested in these assays.

To assess whether the 29 variants also interacted with the nucleotide binding sites of P-glycoprotein, titration experiments using an electron spin resonance (ESR) active ATP analog, SL-ATP were performed, where the amount of P-gp bound SL-ANP was determined in the presence of the 29 variants (Table 5A). The results showed that none of the Group 1 variants significantly affected ATP binding while two of the Group 2 variants reduced ATP binding to about 1 mol per mol of protein. This again indicates that the selection process through ChemGen/selective docking was much more predictive of the effects the potential inhibitors have on the enzyme.

Among all compounds tested, the strongest reversal of MDR and the strongest stimulator of both “basal” and “verapamil-stimulated” ATPase activities was variant 238 of the Group 2 molecules. Compound 238, while strongly reversing MDR, was also the best transport substrate of all the variants tested, a characteristic that is deemed undesirable for any clinically relevant P-gp modulator lead as discussed above.

Group 2 compound 255 reversed MDR and moderately accelerated verapamil-stimulated ATPase rates, an effect that may be related to its role as a transport substrate of P-gp. Compound 280 affected MDR, ATP hydrolysis, as well as SL-ANP binding, but was not strong enough in any of these assays to warrant further study. Finally, Group 2 compound 278 affected MDR relatively weakly, but did inhibit both ATPase hydrolysis and SL-ANP binding to P-gp and therefore may warrant further study. Of the 10 new variants of the P-glycoprotein inhibitor compound 29 that were experimentally tested, compounds 227, 278, 541 and 551 inhibited verapamil-stimulated ATPase activity of the mouse cysteine-less P-gp similarly to that observed with the parental compound 29. However, the results using normal human P-gp reconstituted in a more native-like nanodisc environment indicate that relying on ATP hydrolysis assays, especially when not the same isoform of the enzyme is used and when the enzymes are in different environments (mixed micelles vs. nanodiscs), interpretations of results may not be as clear cut as often assumed.

It was found that the virtual synthesis of hit variants with a program suite like ChemGen combined with a novel selection of characteristics predicted from docking experiments, i.e., increased affinity to targeted structures and decreased affinity against sub-structures that should be avoided as employed here, resulted in efficient and cost-effective identification of five out of five variants assessed that met these goals, was demonstrated in every example tested so date. The comparison of physicochemical characteristics of the resulting new variants and the rational alteration of structure to investigate changed solubility properties (TPSA and log P) without the aid of computational prediction of desired properties, i.e. avoidance of transport structures on P-gp, led to the synthesis of molecules of which a majority was not targeted as desired, while every molecule that underwent the previous selection process inhibited as predicted. This is therefore a good example of how computational evaluation and selection of potential inhibitors before their actual synthesis adds to the speed and overall success rates in identifying hit-to-lead variants that possess desired characteristics.

Materials and methods.

Virtual synthesis of compound 29 derivatives using ChemGen. Because of the nature of the putative allosteric site shown for compound 29 and in light of rational design considerations, only a moderate set of variants were virtually synthesized using the ChemGen program. The ChemGen program and its use are described in detail herein below. In the virtual syntheses performed here, a scaffold molecule, 2-chloro-N-[1-phenyl-3-(2,4,5-trimethylphenyl)-1H-pyrazol-5-yl]acetamide, which is equivalent to the chloroacetamide that retains the “Eastern” substituent group from compound 29 was reacted with approximately 650 thiol compounds obtained by simple structural searches for thiols from the “clean drug-like” commercially available molecule set at the ZINC database³⁴. The 2-chloro-N-[1-phenyl-3-(2,4,5-trimethylphenyl)-1H-pyrazol-5-yl]acetamide scaffold and the thiol precursor molecules were “marked” for reaction in ChemGen as described for the second reaction shown in FIG. 2B. The build process successfully created 647 derivatives of the “Eastern” substituents of 29 which were then geometrically optimized with the electronic Ligand Builder and Optimisation Workbench (eLBOW)—Phenix³⁵ program.

In silico docking of compound 29 variants to a model of human P-gp. AutoDock Vina⁶⁰ and AutoDock 4.2⁶¹⁻⁶³ were used with a model of P-glycoprotein with drug binding domains open to the outside and nucleotide binding sites fully formed that was extracted from targeted molecular dynamics trajectories as described in^{21, 22}. This conformation of P-gp is one that is very similar to the homologous Sav1866 crystal structure reported by Dawson and Locher⁶⁴ and was the conformation with which compound 29 was originally identified²⁶. Ligand docking was limited to a volume equivalent to 20×24×26 Å³ centered on the putative allosteric site of P-gp (see FIG. 1B). 128 replicates for each ligand were calculated and then ranked by the lowest estimated binding energies to the protein target calculated by the docking programs.

Calculation of physicochemical properties. The SWISS-ADME server at http://www.swissadme.ch/ was used for the calculation of the physicochemical properties of the compounds as discussed in⁴⁴.

Imaging of P-glycoprotein and ligands. The VMD (Visual Molecular Dynamics) program suite was used extensively in this work for the analysis of structural data and for the presentation of visual images⁶⁵ and included the SURF surface representation program⁶⁶ as well as the pdb2pgr^{67, 68} and APBS^{69, 70} programs for electrostatic/solvation calculations.

Synthetic procedures: All synthetic procedures and analyses of products are provided herein below.

Cell lines and cell culture. The chemotherapeutic sensitive DU145 human prostate cancer cells⁵² as well as the multidrug resistant sub-line, DU145TXR³³ were generous gifts from Dr. Evan Keller (University of Michigan, Ann Arbor, Mich.). The multidrug resistant DU145TXR was maintained under positive selection pressure by supplementing complete medium with 10 nM paclitaxel (Acros Organics, NJ). Both cell lines were maintained in complete media consisting of RPMI-1640 with L-glutamine, 10% fetal bovine serum (FBS; BioWest, Logan, Utah), 100 U/mL penicillin and 100 μg/mL streptomycin in a humidified incubator at 37° C. and 5% CO₂. The noncancerous human fetal lung cell line, HFL1⁵¹, was kindly provided by Dr. Robert Harrod (Southern Methodist University, Dallas, Tex.) and maintained in complete media consisting of F-12K with L-glutamine, 10% FBS (BioWest, Logan, Utah), 100 U/mL penicillin, and 100 μg/mL streptomycin in a humidified incubator at 37° C. and 5% CO₂. To promote attachment of HFL1 cells, growth surfaces were treated with 0.1 mg/mL rat tail collagen (BD Biosciences, Palo Alto, Calif.) in 0.02 N acetic acid for 10 min and rinsed with PBS prior to use. Cell culture materials were purchased from Corning Inc. (Corning, N.Y.) unless otherwise stated.

MTT cell viability assay. Cells were trypsinized from monolayers and seeded with 3000 cells in 150 μL of complete medium in a 96 well plate. After 24 hours, cells were treated for 48 hours with paclitaxel (Acros Organics, NJ) and/or P-gp inhibitory compounds dissolved in DMSO, or DMSO controls. All additions were diluted into complete medium. After 48 hours of treatment, MTT assays were performed as described⁴¹ using 5 mg/mL of MTT (Acros Organics, NJ) solution prepared in PBS (137 mM NaCl, 2.7 mM KCl, 10 mM Na₂HPO₄, 1.8 mM KH₂PO₄, pH 7.4). After 4 hours of incubation with MTT, the media was removed and the formazan crystals were dissolved in 100 μL of DMSO. The absorbance at 570 nm was then measured using a BioTek Cytation 5 imaging multi-mode reader (Bio-Tek, Winooski, Vt.). Percent viability was calculated using DMSO treated cells as representative for 100% viability, according to Equation 1. Background absorbance was determined using MTT and complete medium without cells and subtracted from all the test values. Equation 1:

$$\% Viability = \frac{Absorbance at 570 nm of test well \times 100}{Absorbance at 570 nm of DMSO treated cells} \qquad ( 1 )$$

The results were plotted as the mean with standard deviation (SD) of eight replicates per concentration from at least two independent experiments with n=8. The graphical representations and IC₅₀ values were determined using four parameter variable slope non-linear regression, using the following equation: Y=bottom+(top-bottom)/(1+10\hat{ }((log IC50−X)*Hill Slope) (GraphPad Prism™, La Jolla Calif., USA, Version 6.05). The reported “fold sensitization” was calculated as follows, per Equation 2:

$$Fold sensitization = \frac{\begin{matrix}{IC}_{50} value of A 2780 ADR cells treated \\ with chemotherapeutic only\end{matrix}}{\begin{matrix}\begin{matrix}{IC}_{50} value of A 2780 ADR or A 2780 \\ cells treated with chemotherapeutic\end{matrix} \\ and P \text{-} gp inhibitory compound\end{matrix}} \qquad ( 2 )$$

Calcein AM assay. To assess inhibition of P-gp-catalyzed transport of the P-gp pump substrate, calcein AM, DU145TXR cells were seeded in 96 wells plates and allowed to grow in complete medium until confluency was reached. Medium was removed, and cells were treated without 2 sM P-gp inhibitory compounds and 1 μg/mL calcein AM (Life Technologies, OR) and diluted into phenol red free RPMI 1640 media. To study the effect of pre-incubation of compounds, cells were treated with just P-gp inhibitory compounds and incubated at 37° C. for six hours before adding calcein AM. Fluorescence excitation at 485 nm with a 20 nm gate and at emission at 535 nm with a 20 nm gate was measured using a BioTek Cytation 5 imaging multi-mode reader (Bio-Tek, Winooski, Vt.) over 60 minutes in 20 minute intervals. DMSO was used as vehicle. Results were plotted as the mean with standard deviation (SD) of six replicates per concentration and are representative of at least two independent experiments.

Cellular Accumulation Assays for Experimental P-gp Inhibitors. Cells used (DU145TXR), cell culturing, cell exposure to compounds, cellular handling and extractions were performed as described in²⁸. LC-MS/MS methods were performed as described in⁷¹ and as modified in²⁸.

P-glycoprotein Purification. Cysteine-less MDR3 and the human normal MDR1 P-glycoprotein was recombinantly expressed in the yeast Pichia pastoris essentially as in^{45, 46} and used for assaying ATP hydrolysis and ATP binding to the protein in the presence of the 29-variants. Purification of the protein was performed as described⁴⁹ with small modifications resulting in highly enriched P-gp in mixed micelles containing dodecyl maltoside (DDM) and lysophosphatidyl choline²⁶.

Nanodisc assembly. Human P-gp in mixed detergent micelles obtained during protein purification, was reconstituted into nanodiscsas described in references^{73, 74} with small modifications. P-gp was assembled with membrane scaffold protein, MSP1E3D1 (Sigma-Aldrich) expressed in BL21 (DE3) and L-alpha-phosphatidylcholine (Sigma-Aldrich), at a ratio of 1:10:500 (P-gp:MSP:PC) in 50 mM Tris-CL (pH 8). The detergent was removed with Bio-Beads™ SM-2 Adsorbent Media (BioRad). Ni-NTA Agarose (Qiagen) chromatography was used to purify P-gp reconstituted nanodiscs using 6 bed volumes of start buffer (20% (v/v) glycerol, 50 mM Tris-CL pH 7.5 at 4° C., 50 mM NaCl), and 5 bed volumes of elution buffer (20% (v/v) glycerol, 50 mM Tris-Cl pH 7.5 at 4° C., 50 mM NaCl, 300 mM imidazole).

ATPase Activity Assays. ATP hydrolysis activity was measured using a coupled enzyme assay⁷² as modified in reference²⁶. The specific basal activity of the mouse MDR3 cysteineless P-gp was between 20 and 30 nmol min⁻¹mg⁻¹, and transport substrate (verapamil) stimulated activity was 200-400 nmol min⁻¹mg⁻¹. The specific basal activity of normal human MDR1 P-gp was between 123 and 193 nmol min⁻¹ mg⁻¹, and transport substrate (verapamil) stimulated activity was between 193-263 nmol min⁻¹mg⁻¹.

ESR Measurements. ESR measurements were as described in²⁶. The amount of protein-bound spin-labeled (SL) adenine nucleotide was determined as the difference between the known total concentration of SL-ATP (2′,3′-(2,2,5,5,-tetramethyl-3-pyrroline-1-oxyl-3-carboxylic acid ester) —ATP⁴⁸) added and the free spin-labeled nucleotide (SL-ANP) observed in the experiment. Hyperbolic curve fitting of the results was performed using GraphPad Prism 7 to determine maximum binding and apparent affinity for the spin-labeled nucleotide. The equation used for the fitting the curves was y=P*x/(P2+x), where P1 corresponds to the maximum binding of SL-ANP (moles of SL-ANP bound per mole P-gp), and P2 equals the apparent dissociation constant for SL-ANP. To quantify the amount of free SL-ANP, standard curves were established where the signal amplitude of the high field signal of the ESR spectra of free SL-ANP in the absence of protein was correlated to the concentration of SL-ANP added. All ESR measurements were performed using a Bruker EMX 6/1 ESR spectrophotometer operating in X-band mode and equipped with a high sensitivity cavity. Spectra were acquired at a microwave frequency of 9.33 GHz, microwave power of 12.63 mW, 100 kHz modulation frequency and a resolution of 1024 points. The centerfield of the scan was at 3325 G and an area of 100 G was scanned. The peak to peak modulation amplitude was 1G and the time constant was set to 10.240 ms. The conversion time was 163.84 ms, resulting in a total time sweep of 167.772 s. The signal gain was adjusted for the SL-ATP concentrations in the different experiments.

Synthetic Procedures. General Materials and Methods.

The reactions were performed under nitrogen and dried glassware. Reagents were purchased from Sigma-Aldrich (St. Louis, Mo.), Alfa Aesar (Ward Hill, Mass.), EMD Millipore (Billerica, Mass.), Oakwood Chemical (West Columbia, S.C.), and Cayman Chemical (Ann Arbor, Mich.). Silica gel P60 (SiliCycle) was used for column chromatography and Analytical Chromatography TLC Silica gel 60 F₂₅₄ (Merck Millipore, Darmstadt, Germany) was used for analytical thin layer chromatography. ¹H NMR and ¹³C NMR spectra were used for analyzed the compounds by using CDCl₃ (Cambridge Isotope Laboratories, Cambridge, Mass.) on a JEOL 500 MHz and BRUKER 400 MHz spectrometer in the Department of Chemistry at Southern Methodist University. Chemical abbreviations are used as follows: CH₂Cl₂, dichloromethane; EtOAc, ethyl acetate; THF, tetrahydrofuran; DMF, dimethylformamide; H₂O, water; HBTU, O-benzotriazole-N,N,N′,N′-tetramethyl-uronium-hexafluoro-phosphate; DIPEA, N,N-diisopropylethylamine; KOH potassium hydroxide; DMSO, dimethylsulfoxide N₂, nitrogen. High resolution mass spectroscopy was performed on a Shimadzu IT-TOF (ESI source) and low resolution mass spectroscopy was performed on a Shimadzu LCMS-8050 Triple Quadrupole LCMS (ESI source) or a Shimadzu Matrix Assisted Laser Desorption/Ionization MS (MALDI) at the Shimadzu Center for Advanced Analytical Chemistry at the University of Texas, Arlington.

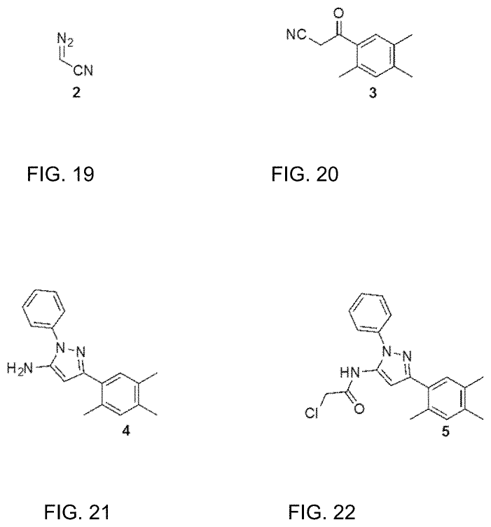

FIG. 19 shows Diazoacetonitrile (compound 2)¹¹ Aminoacetonitrile hydrochloride (0.575 g, 6.21 mmol, 1.0 equiv) was dissolved in water (10 mL) and CH₂Cl₂, and then placed in an ice bath. The mixture was stirred and then sodium nitrite (0.43 g, 6.21 mmol, 1.0 equiv) was added over 5 min. After 15 min, the reaction mixture was extracted with CH₂Cl₂, washed with brine, and dried with Na₂SO₄. The solution was filtered and used directly in the next step using an estimated yield of 30%.

FIG. 20 shows 3-oxo-3-(2,4,5-trimethylphenyl)propanenitrile (compound 3)¹¹ 2,4,5-trimethylbenzaldehyde 1 (183 mg, 1.24 mmol, 1.50 equiv) was dissolved in a minimum amount of CH₂Cl₂ and added to a solution of diazoacetonitrile 2 (1.9 mmol, 1 equiv) in dichloromethane. BF₃.OEt₂ (0.4 mmol, 0.004 mL, 0.2 equiv) was added dropwise to the reaction mixture until gas stopped evolving and the color was changed from light green to dark red. After 20 min, the reaction mixture was concentrated and purification done by silica column chromatography using (1:5 Ethyl Acetate: Hexane) to give a tan solid (162 mg, 0.86 mmol, 70% yield). ¹H NMR (500 MHz, CDCl₃) δ 7.36 (s, 1H), 7.06 (s, 1H), 4.02 (s, 2H), 2.50 (s, 3H), 2.28 (s, 6H). ¹³C NMR (125 MHz, CDCl₃) δ 19.4, 20.0, 21.6, 31.3, 114.4, 130.9, 131.3, 134.3, 134.5, 138.4, 143.2, 188.7.

FIG. 21 shows 1-phenyl-3-(2,4,5-trimethylphenyl)-1H-pyrazol-5-amine (compound 4) Compound 4 was prepared by adaptation of a literature procedure¹². Benzoylacetonitrile 3 (250 mg, 1.33 mmol, 1.00 equiv) and phenylhydrazine (0.31 mL, 1.3 mmol, 1.0 equiv) were added to pressure tube and heated to 165° C. for six hours. The mixture was purified by silica column chromatography using CH₂Cl₂ to give a yellow solid (258 mg, 0.931 mmol, 70% yield). ¹H NMR (500 MHz, CDCl₃) δ 7.66 (m, 2H), 7.48 (m, 2H), 7.42 (m, 1H), 7.32 (m, 1H), 7.00 (s, 1H), 5.81 (s, 1H), 3.82 (s, 2H), 2.46 (s, 3H), 2.24 (s, 6H); ¹³C NMR (125 MHz, CDCl₃) δ 18.7, 19.5, 20.4, 91.4, 123.6, 126.9, 129.5, 130.2, 130.5, 132.2, 133.2, 133.5, 135.9, 139.1, 144.6, 152.4; HRMS calcd for C₁₈H₁₉N₃ (M+H)⁺ 278.1652, found 278.1650.

FIG. 22 shows 2-chloro-N-[1-phenyl-3-(2,4,5-trimethylphenyl)-1H-pyrazol-5-yl]acetamide (compound 5) Pyrazole 4 (530 mg, 1.93 mmol, 1.00 equiv) was dissolved in CH₂Cl₂ and placed in an ice bath. Chloroacetyl chloride (260 mg, 2.30 mmol, and 0.22 mL) was added dropwise. The reaction mixture was stirred overnight at room temperature. The reaction washed with H₂O and brine, dried over Na₂SO₄, filtered and concentrated. The product was obtained as a brown crystal (660 mg, 1.87 mmol, 97% yield) and used without any further purification. ¹H NMR (500 MHz, CDCl₃) δ 8.77 (s, 1H), 7.53 (m, 4H), 7.43 (s, 2H), 7.03 (s, 1H) 6.90 (s, 1H), 4.15 (s, 2H), 2.48 (s, 3H), 2.25 (s, 6H); ¹³C NMR (125 MHz, CDCl₃) δ 19.3, 19.5, 20.7, 42.7, 98.7, 124.6, 128.7, 129.6, 129.8, 130.2, 132.2, 133.4, 133.2, 134.0, 134.5, 136.6, 137.4, 152.8, 162.6, 169.2; HRMS calcd for C₂₀H₂₀N₃OCl (M+H)⁺ 354.1368, found 354.1371.

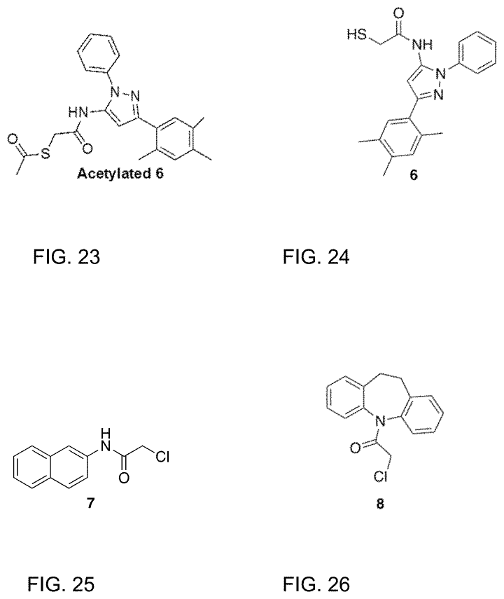

FIG. 23 shows 2-(acetylsulfanyl)-N-[1-phenyl-3-(2,4,5-trimethylphenyl)-1H-pyrazol-5-yl]acetamide (compound acetylated 6) Chloride 5 (224 mg, 0.640 mmol, 1.00 equiv) was dissolved in 3 mL of anhydrous THF and placed in an ice bath. KSAc (143 mg, 1.28 mmol) was then added. The reaction was stirred overnight at room temperature. An orange solid was obtained (243 mg, 0.62 mmol, 97% yield). ¹H NMR (500 MHz, CDCl₃) δ 8.38 (s, 1H), 7.51 (m, 4H), 7.42 (m, 2H), 7.08 (s, 1H), 6.85 (s, 1H), 3.59 (s, 2H), 2.48 (s, 3H), 2.32 (s, 6H), 2.25 (s, 3H); ¹³C NMR (125 MHz, CDCl₃) δ 19.3, 19.4, 20.6, 30.2, 33.2, 98.9, 129.8, 133.4, 133.9, 135.4, 136.4, 137.9, 152.4, 165.2, 196.3; HRMS calcd for C₂₂H₂₃N₃O₂S (M+H)⁺ 394.1584, found 394.1576.

FIG. 24 shows 2-mercapto-N-(1-phenyl-3-(2,4,5-trimethylphenyl)-1H-pyrazol-5-yl)acetamide (compound 6) In a dry Schlenk flask, the thioester Acetylated 6 (80 mg, 0.2 mmol, 1 equiv) was dissolved in 3 mL anhydrous CH₃OH and then 0.8 mL of 2 M NaOH (16 mg, 1.6 mmol, 8.0 equiv) was added. The reaction was degassed using a freeze-pump-thaw procedure. The solvent and reactants in the Schlenk flask were frozen by submerging in liquid nitrogen. Then, the flask was opened to the vacuum for one minute. After that, the flask was sealed and allowed to warm up until the solvent has completely become liquid again. This procedure has repeated two times. After the last cycle was complete, the flask was brought to room temperature and filled with N₂. After stirring one hour, the reaction mixture was concentrated and diluted with ethyl acetate, and then acidified to pH=1 by using HCl. The extraction was done by using ethyl acetate/water and dried over Na₂SO₄. The purification was done by silica column chromatography using 1:2 (Ethyl Acetate: Hexane) to obtain white crystals (49 mg, 70% yield). ¹H NMR (500 MHz, CDCl₃) δ 8.94 (s, 1H), 7.52 (m, 4H), 7.40 (m, 2H), 7.00 (s, 1H), 6.90 (s, 1H), 3.36 (s, 2H), 2.47 (s, 6H), 2.24 (s, 3H), 1.8 (s, 1H); ¹³C NMR (125 MHz, CDCl₃) δ 19.2, 19.7, 21.1, 28.6, 98.1, 124.7, 128.6, 130.0, 130.3, 132.3, 133.3, 133.9, 135.2, 136.4, 138.0, 148.5, 152.7, 165.5; HRMS calcd for C₂₀H₂₁N₃OS (M+H)⁺ 352.1478, found 352.1481.

### General Procedure for the Synthesis of 2-Chloro-Acetamide Derivatives

Each substituted amine (1.0 equiv) was dissolved in 3 mL of anhydrous THF, followed by addition of one equivalent of Et₃N. After placing the reaction mixture in an ice bath, 2-chloroacetyl chloride (1.2 equiv) was added dropwise for one hour and the reaction was stirred overnight at room temperature. After being concentrated, CH₂Cl₂ and water were added and the organic compounds were extracted three times with CH₂Cl₂. The organic layers were washed with brine, dried over Na₂SO₄, filtered, and concentrated.

FIG. 25 shows 2-chloro-N-(naphthalen-2-yl) acetamide (compound 7)¹³ Light orange solid (112 mg, 72% yield). ¹H NMR (500 MHz, CDCl₃) δ 8.44 (s, 1H), 8.25 (s, 1H), 7.80 (m, 3H), 7.50 (m, 3H), 4.24 (s, 2H).

FIG. 26 shows 2-chloro-1-(10,11-dihydro-5H-dibenzo[b]azepin-5-yl)ethan-1-one (compound 8)¹⁴. White solid (199 mg, 72% yield). ¹H NMR (500 MHz, CDCl₃) δ 7.45-7.25 (m, 4H), 7.08 (m, 2H), 6.79 (m, 2H), 4.13 (t, 2H, J=12.6 Hz), 4.02 (t, 2H, J=12.6 Hz), 3.50 (m, 2H), 3.35 (m, 2H), 3.10 (s, 2H).

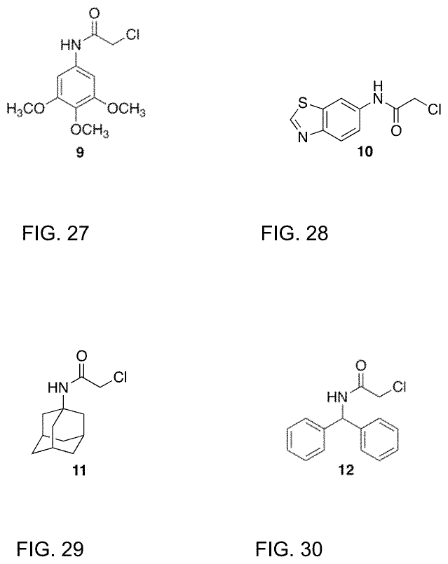

FIG. 27 shows 2-chloro-N-(3,4,5-trimethoxyphenyl)acetamide (compound 9)¹⁵. White solid (212 mg, 75% yield). ¹H NMR (500 MHz, CDCl₃) δ 8.24 (s, 1H), 6.81 (s, 2H), 4.14 (s, 2H), 3.79 (s, 9H).

FIG. 28 shows N-(benzo[d]thiazol-6-yl)-2-chloroacetamide (compound 10)¹⁶. White solid (56 mg, 75% yield). ¹H NMR (500 MHz, CDCl₃) δ 8.94 (s, 1H), 8.54 (s, 1H), 8.45 (br s, 1H), 8.11 (d, 1H, J=9.1 Hz), 7.42 (d, 1H, J=9.1 Hz), 4.23 (s, 1H).

FIG. 29 shows N-((3s,5s,7s)-adamantan-1-yl)-2-chloroacetamide (compound 11)¹⁷. White solid (0.97 g, 75% yield). ¹H NMR (500 MHz, CDCl₃) δ 6.22 (s, 1H), 3.90 (s, 2H), 1.99 (m, 9H), 1.66 (m, 6H).

FIG. 30 shows N-benzhydryl-2-chloroacetamide (compound 12)¹⁸. The mixture was purified by silica column chromatography using 1:4 (Ethyl Acetate: Hexane) to give a white solid (0.81 g, 56% yield). ¹H NMR (500 MHz, CDCl₃) δ 7.34 (m, 6H), 7.30 (d, 1H, J=12.6 Hz), 7.24 (m, 4H), 6.25 (d, 1H, J=9.2 Hz), 4.12 (s, 2H).

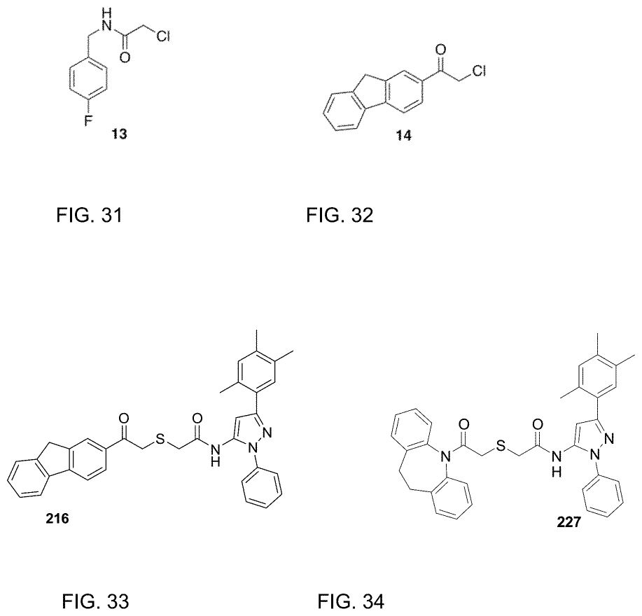

FIG. 31 shows 2-chloro-N-(4-fluorobenzyl)acetamide (compound 13)¹⁹. White solid (241 mg, 75% yield). ¹H NMR (500 MHz, CDCl₃) δ 7.24 (m, 2H), 6.98 (m, 2H), 6.88 (s, 1H), 4.40 (s, 2H), 4.06 (s, 2H).

FIG. 32 shows 2-chloro-1-(9H-fluoren-2-yl)ethan-1-one (compound 14)²⁰. 9-fluorene (100 mg, 0.6 mmol, 1 equiv) was dissolved in 100 mL of methylene chloride. After the reaction mixture was cooled to 0° C., anhydrous aluminum chloride (120 mg, 0.9 mmol, 1.5 equiv) was added. The reaction was stirred for 15 min. Chloroacetyl chloride (102 mg, 0.9 mmol, 1.5 equiv) was added in dropwise. After 15 min at 0° C. and 45 min of stirring at room temperature, the reaction mixture was poured into a mixture of 500 mL of ice and 100 mL of hydrochloric acid. The organic phase was extracted and washed with brine. The product was dried over sodium sulfate collected after concentrated. The product was obtained as white solid (140 mg, 96% yield). ¹H NMR (500 MHz, CDCl₃) δ 8.13 (m, 1H), 7.96 (m, 1H), 7.82 (m, 1H), 7.56 (m, 1H), 7.40 (m, 3H), 4.73 (s, 2H), 3.93 (s, 2H).

General Synthesis for S_{N}2 Coupling of Alkyl Thiols.

The reaction was performed by dissolving the thiol (1 equiv) in 3 mL of DMF (deoxygenated by bubbling N₂) and adding K₂CO₃ (2 equiv). The chloroacetamide (1.2 equiv) was then added to the reaction mixture and stirred overnight at room temperature. The reaction was diluted in EtOAc and washed with water. The water layer was extracted three times with EtOAc, washed with brine, dried over Na₂SO₄, filtered and concentrated to give the crude product, which was purified as indicated.

FIG. 33 shows 2-{[2-(9H-flouoren-2-yl)-2-oxoethyl]}-N-[1-phenyl-3-(2,4,5-trimethylphenyl0-1H-pyrazol-5-yl]acetamide (compound 216) The mixture was purified by silica column chromatography using (1:3 Ethyl Acetate: Hexane) to give a white solid (0.017 g, 50% yield). ¹H NMR (500 MHz, CDCl₃) δ 8.94 (s, 1H), 8.55 (s, 1H), 8.09 (s, 1H), 7.73 (m, 3H), 7.52-7.44 (m, 8H), 7.00 (s, 1H), 6.82 (s, 1H), 3.4 (s, 2H), 3.30 (s, 2H), 2.42 (s, 3H), 2.24 (s, 6H); ¹³C NMR (125 MHz, CDCl₃) δ 193.4, 165.2, 152.3, 147.4, 144.5, 143.4, 140.0, 137.8, 136.1, 135.3, 133.6, 133.1, 132.7, 132.1, 130.17, 129.9, 128.3, 128.0, 127.9, 127.1, 125.2, 125.1, 121.0, 119.8, 98.7, 60.3, 53.3, 37.9, 36.7, 36.2, 20.6, 19.3; HRMS calculated for C₃₅H₃₁N₃O₂S (M+H)⁺ 558.2210, found 558.2201.

FIG. 34 shows 2-[(2-{2-azatricyclo[9.4.0.0]pentadeca-1(11),3(8},4,6,12,14-hexaen-2-yl)-2-oxoethyl)sulfanyl]-N-[1-phenyl-3-(2,4,5-trimethylphenyl)-1H-pyrazol-5-yl]acetamide (compound 227) The mixture was purified by silica column chromatography using (1:2 Ethyl Acetate: Hexane) to give a white solid (0.013 g, 60% yield). ¹H NMR (500 MHz, CDCl₃) δ 9.57 (s, 1H), 7.52 (m, 2H), 7.42 (m, 5H), 7.23-7.25 (m, 5H), 7.01 (s, 2H), 6.85 (s, 2H), 3.40 (s, 2H), 3.30 (s, 2H), 2.88 (m, 4H), 2.45 (s, 3H), 2.22 (s, 6H); ¹³C NMR (125 MHz, CDCl₃) δ 168.8, 166.5, 152.3, 141.0, 139.2, 138.2, 137.7, 136.2, 135.9, 134.6, 133.3, 132.2, 130.9, 129.4, 128.1, 127.7, 127.2, 126.6, 125.5, 124.6, 99.7, 35.62, 32.1, 29.7, 28.4, 20.7, 20.5, 18.9; HRMS calculated for C₃₆H₃₄N₄O₂S (M+H)⁺ 587.2475, found 587.2477.

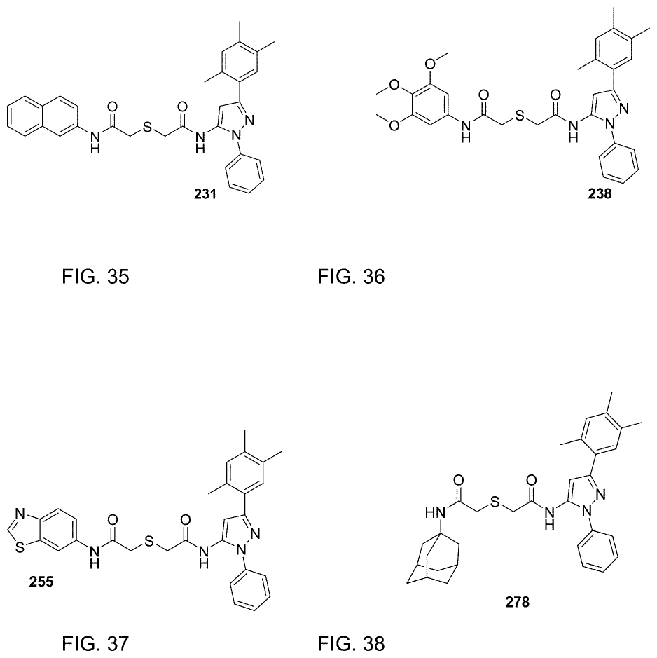

FIG. 35 shows 2-({[(naphthalen-2-yl)carbamoyl]methyl}sulfanyl)-N-[1-phenyl-3-(2,4,5-trimethylphenyl)-1H-pyrazol-5-yl]acetamide (compound 231) The mixture was purified by silica column chromatography using (1:3 Ethyl Acetate: Hexane) to give a white solid (0.019 g, 50% yield). ¹H NMR (500 MHz, CDCl₃) δ 9.12 (s, 1H), 8.03 (s, 1H), 7.88 (m, 1H), 7.84 (m, 1H), 7.81 (m, 1H), 7.59 (m, 1H), 7.50 (m, 2H), 7.42 (m, 6H), 7.35 (m, 1H), 7.03 (s, 1H), 6.91 (s, 1H), 3.93 (s, 2H), 3.41 (s, 2H), 2.49 (s, 3H), 2.25 (s, 6H); ¹³C NMR (125 MHz, CDCl₃) δ 166.7, 166.2, 152.4, 137.9, 136.3, 135.1, 134.6, 133.6, 133.1, 132.2, 130.8, 130.2, 129.8, 129.7, 128.9, 128.7, 128.3, 127.7, 127.6, 127.1, 126.6, 125.2, 125.1, 121.0, 119.8, 117.0, 99.6, 36.6, 36.0, 20.6, 19.3, 19.0; HRMS calculated for C₃₂H₃₀N₄O₂S (M+H)⁺ 535.2162, found 535.2157.

FIG. 36 shows N-[1-phenyl-3-(2,4,5-trimethylphenyl)-1H-pyrazol-5-yl]-2-({[(3,4,5-trimethoxyphenyl)carbamoyl]methyl}sulfanyl)acetamide (compound 238) The mixture was purified by silica column chromatography using (2:1 Ethyl Acetate: Hexane) to give a white solid (0.016 g, 62% yield). ¹H NMR (500 MHz, CDCl₃) δ 8.67 (s, 1H), 8.35 (s, 1H), 7.53 (m, 2H), 7.50 (m, 2H), 7.40 (m, 2H), 7.04 (s, 1H), 6.85 (s, 1H), 6.80 (s, 2H), 3.78 (s, 9H), 3.41 (s, 2H), 3.27 (s, 2H), 2.46 (s, 3H), 2.25 (s, 6H); ¹³C NMR (125 MHz, CDCl₃) 166.6, 153.2, 152.6, 138.0, 134.9, 133.7, 133.3, 128.3, 124.9, 100.0, 97.6, 61.0, 56.1, 36.6, 36.0, 20.6, 19.2; HRMS calculated for C₃₁H₃₄N₄O₅S (M+H)⁺ 575.2323, found 575.2329.

FIG. 37 shows 2-({[(1,3-benzothiazol-6-yl)carbamoyl]methyl}sulfanyl)-N-[1-phenyl-3-(2,4,5 trimethylphenyl)-1H-pyrazol-5-yl]acetamide (compound 255) The mixture was purified by silica column chromatography using (2:1 Ethyl Acetate: Hexane) to give a white solid (0.005 g, 49% yield). ¹H NMR (500 MHz, CDCl₃) δ 8.91 (s, 1H), 8.57 (m, 2H), 8.45 (s, 1H), 8.04 (s, 1H), 7.55 (m, 5H), 7.39 (m, 2H), 7.01 (s, 1H), 6.88 (s, 1H), 3.47 (s, 2H), 3.36 (s, 2H), 2.47 (s, 3H), 2.25 (s, 6H); ¹³C NMR (125 MHz, CDCl₃) δ 167.2, 166.1, 153.9. 152.5, 150.26, 138.0, 136.4, 135.2, 133.9, 133.2, 132.5, 130.5, 129.5, 128.3, 125.2, 123.7, 119.2, 112.8, 99.6, 36.7, 36.1, 20.7, 19.2; HRMS calculated for C₂₉H₂₇N₅O₂S₂ (M+H)⁺ 542.1679, found 542.1661.

FIG. 38 shows 2-({[(adamantan-1-yl)carbamoyl]methyl}sulfanyl)-N-[1-phenyl-3-(2,4,5-trimethylphenyl)-1H-pyrazol-5-yl]acetamide (compound 278) The mixture was purified by silica column chromatography using (1:2 Ethyl Acetate: Hexane) to give a white solid (0.017 g, 57% yield). ¹H NMR (500 MHz, CDCl₃) δ 9.44 (s, 1H), 7.53 (m, 2H), 7.42 (m, 2H), 7.35 (m, 2H), 7.00 (s, 1H), 6.79 (s, 1H), 3.29 (s, 2H), 2.93 (s, 2H), 2.40 (s, 3H), 2.20 (s, 6H), 1.98 (s, 3H), 1.83 (m, 6H), 1.57 (m, 6H); ¹³C NMR (125 MHz, CDCl₃) δ 167.6, 166.2, 152.4, 138.3, 136.3, 135.7, 133.9, 133.3, 132.2, 130.2, 128.0, 99.2, 41.4, 36.4, 29.5, 20.5, 19.2; HRMS calculated for C₃₂H₃₈N₄O₂S (M+H)⁺ 543.2788, found 543.2789.

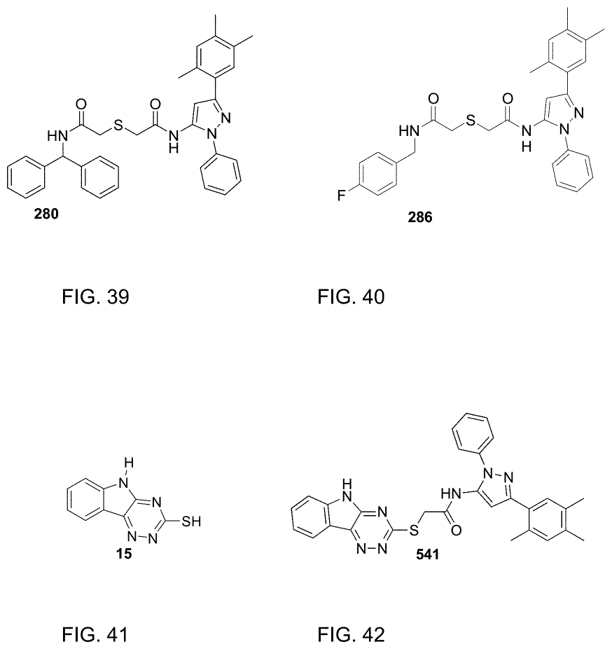

FIG. 39 shows N-(diphenylmethyl)-2-[({[1-phenyl-3-(2,4,5-trimethylphenyl)-1H-pyrazol-5-yl]carbamoyl)methyl}sulfanyl]acetamide (compound 280) The mixture was purified by silica column chromatography using (1:4 Ethyl Acetate: Hexane) to give a white solid (0.017 g, 52% yield). ¹H NMR (500 MHz, CDCl₃) δ 9.11 (s, 1H), 7.48 (m, 2H), 7.40 (m, 3H), 7.29 (m, 8H), 7.18 (m, 3H), 7.02 (m, 1H), 6.80 (s, 1H), 6.08 (d, 1H, J=8.5 Hz), 3.26 (s, 2H), 3.13 (s, 2H), 2.48 (s, 3H), 2.25 (s, 6H); ¹³C NMR (125 MHz, CDCl₃) δ 168.0, 166.2, 152.3, 140.7, 138.0, 136.4, 135.6, 129.5, 128.7, 127.2, 125.3, 124.8, 99.6, 57.5, 35.7, 35.1, 20.7, 19.1; HRMS calculated for C₃H₃₄N₄O₂S (M+H)⁺ 575.2475, found 575.2472.

FIG. 40 shows N-[(4-fluorophenyl)methyl]-2-[({[1-phenyl-3-(2,4,5-trimethylphenyl)-1H-pyrazol-5-yl]carbamoylmethyl}sulfanyl]acetamide (compound 286) The mixture was purified by silica column chromatography using (1:3 Ethyl Acetate: Hexane) to give a white solid (0.014 g, 54% yield). ¹H NMR (500 MHz, CDCl₃) δ 9.05 (s, 1H), 7.55 (m, 2H), 7.47 (m, 2H), 7.38 (m, 2H), 7.19 (m, 2H), 6.99 (m, 3H), 6.83 (m, 1H), 6.47 (s, 1H), 4.30 (m, 1H), 3.38 (s, 2H), 3.13 (s, 2H), 2.49 (s, 3H), 2.23 (s, 6H); ¹³C NMR (125 MHz, CDCl₃) δ 168.4, 166.1, 152.5, 138.0, 136.5, 135.5, 133.4, 132.4, 130.4, 129.7, 128.4, 125.1, 115.8, 99.5, 43.2, 36.2, 35.6, 20.7, 19.5, 19.2; HRMS calculated for C₂₉H₂₉N₄O₂FS (M+H)⁺ 517.2068, found 517.2068.

Synthetic Procedures for Aromatic Sulfide Derivatives.

FIG. 41 shows 5H-[1,2,4]triazino[5,6-b]indole-3-thiol (compound 15)²¹ In a round-bottom flask, isatin (200 mg, 1.36 mmol, 1 equiv) and potassium carbonate were dissolved 5 mL of water. (124 mg, 1.36 mmol, 1 equiv) of thiosemicarbazide was added to a solution. The reaction was reflux for 16 h. The reaction mixture was acidified by using 0.5 mL acetic acid. The yellow precipitate was afforded and washed with water and acetic acid (12:1). The yellow solid was triturated with hot DMF. The product was filtered and dried under high vacuum to give a yellow solid (119 mg, 0.59 mmol, 43% yield). ¹H NMR (500 MHz, DMSO-D6) δ 7.99 (d, 1H, J=8.0 Hz), 7.94 (s, 1H), 7.61 (t, 1H, J=8.0 Hz), 7.42 (d, 1H, J=8.9 Hz), 7.33 (t, 1H, J=8.0 Hz); ¹³C NMR (125 MHz, CDCl₃) δ 113.4, 118.4, 122.3, 123.5, 132.3, 135.9, 143.6, 149.6, 179.6; HRMS calculated for C₉ H₆ N₄S (M+H)+203.0389, found 203.0386

FIG. 42 shows 2-((5H-[1,2,4]triazino[5,6-b]indol-3-yl)thio)-N-(1-phenyl-3-(2,4,5-trimethylphenyl)-1H-pyrazol-5-yl)acetamide (compound 541) The reaction was performed by dissolving the thiol 15 (10 mg, 0.049 mmol, 1.0 equiv) in 3 mL of methanol and adding triethyl amine (1.5 equiv). The chloroacetamide (1.0 equiv) was added to the reaction mixture and stirred overnight at room temperature. The reaction mixture was filtered and washed with methanol. The product was collected after high vac as a yellow powder (15 mg, 0.028 mmol, 58% yield). ¹H NMR (500 MHz, CDCl₃) δ 10.37 (s, 1H), 8.29 (d, 1H, J=10.8), 7.66 (m, 1H), 7.56 (m, 3H), 7.41 (m, 1H), 7.32 (m, 4H), 7.19 (m, 1H), 6.96 (s, 1H), 6.64 (s, 1H), 4.19 (s, 2H), 2.39 (s, 3H), 2.15 (s, 6H). ¹³C NMR (500 MHz, DMSO-D6) δ 18.6, 20.3, 39.5, 102.0, 112.5, 116.7, 121.1, 122.7, 123.9, 126.8, 128.7, 129.5, 130.8, 140.3, 146.4, 150.9, 166.0, 166.9; HRMS calculated for C₂₉H₂₅N₇OS (M+H)+520.1919, found 520.1914.

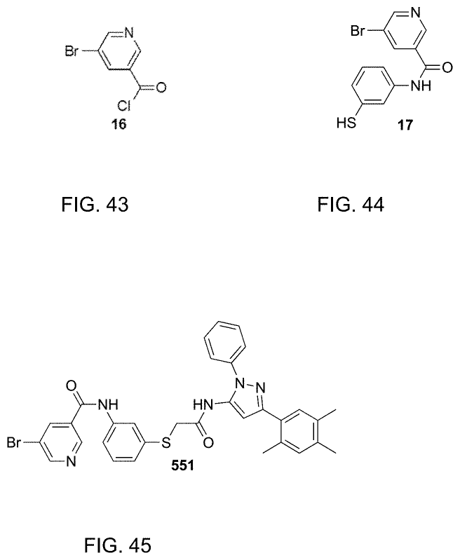

FIG. 43 shows 5-bromonicotinoyl chloride (compound 16) 5-Bromonicotinic acid (100 mg, 0.5 mmol, 1.00 equiv) was dissolved in 3 mL of 1,2-dichloroethane. Thionyl chloride (0.11 mL, 1.5 mmol, 3 equiv) was added to the reaction mixture followed by one drop of DMF. The reaction was heated and reflux for overnight. The reaction was stopped and cooled to room temperature. The excess thionyl chloride was removed under reduced pressure to give a carbonyl chloride that was used for the next step without further purification.

FIG. 44 shows 5-bromo-N-(3-mercaptophenyl)nicotinamide (compound 17)²² 3-Aminothiophenol (46 mg, 0.37 mmol, 1.0 equiv) was dissolved in 5 mL of dichloromethane. The carbonyl chloride 16 (82 mg, 0.37 mmol, 1.0 equiv) and pyridine (0.044 mL, 0.55 mmol, 1.5 equiv) were added to the solution at −10° C. and the reaction was stirred overnight at room temperature. The reaction mixture was washed with 10 mL of 1M HCl and the solvent was removed under reduced pressure. The solid was dissolved in 2:1 methanol and water. Potassium carbonate (51 mg, 0.37 mmol, 1.0 equiv) was added and the reaction mixture was stirred for one hour at room temperature. The crude mixture was acidified to pH=1 by using 1M HCl. The methanol was removed under reduced pressure and the aqueous layer was extracted by dichloromethane. The organic layer was washed with brine, dried over Na₂SO₄, filtered, and concentrated. The mixture was purified by silica column chromatography using (2:1 Ethyl Acetate: Hexane) to give a white solid (40 mg, 0.129 mmol, 35% yield). ¹H NMR (500 MHz, CDCl₃) δ 8.96 (s, 1H), 8.83 (s, 1H), 8.31 (s, 1H), 7.99 (s, 1H), 7.65 (s, 1H), 7.31 (d, 1H, J=8.6), 7.26 (m, 1H), 7.07 (d, 1H, J=8.6), 3.53 (s, 1H); ¹³C NMR (125 MHz, CDCl₃) δ 117.2, 120.3, 124.8, 129.6, 132.4, 137.8. 139.3, 147.5, 153.2, 162.5.

FIG. 45 shows 5-bromo-N-(3-((2-oxo-2-((1-phenyl-3-(2,4,5-trimethylphenyl)-1H-pyrazol-5-yl)amino)ethyl)thio)phenyl)nicotinamide (compound 551). The thiol 17 (22 mg, 0.071 mmol, 1 equiv) was dissolved in 3 mL of DMF (deoxygenated by bubbling N₂) and K₂CO₃ (22 mg, 0.163 mmol, 2.3 equiv) was added. The chloroacetamide (25 mg, 0.071 mmol, 1.0 equiv) was added to the reaction mixture and stirred overnight at 110° C. The reaction was diluted in EtOAc and washed with water. The organic layer was washed with brine, dried over Na₂SO₄, filtered and concentrated. The purification was done by using (2:1 Ethyl acetate: Hexane) to give (23 mg, 0.037 mmol, 51% yield). ¹H NMR (500 MHz, CDCl₃) δ 8.98 (s, 1H), 8.90 (s, 1H), 8.74 (s, 1H), 8.26 (s, 2H), 7.46 (m, 2H), 7.32-7.23 (m, 5H), 6.93 (m, 2H), 6.88 (s, 1H), 6.80 (s, 1H), 3.73 (s, 2H), 2.37 (s, 3H), 2.13 (s, 6H); ¹³C NMR (125 MHz, CDCl₃) δ 19.2, 19.5, 20.7, 37.2, 98.4, 118.7, 118.9, 124.6, 128.4, 130.0, 130.3, 133.2, 137.8, 138.1, 146.0, 152.6, 153.7, 162.6, 164.7; HRMS calculated for C₃₂H₂₈NSO₂SBr (M+H)+ 626.1218, found 626.1220.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

### REFERENCES

Brewer, F. K., Follit, C. A., Vogel, P. D., & Wise, J. G. (2014). In silico Screening for Inhibitors of P-Glycoprotein that Target the Nucleotide Binding Domains. Molecular pharmacology, 86(6), 716-726.
Cramer, R. D., Patterson, D. E., Clark, R. D., Soltanshahi, F. & Lawless, M. S. Virtual compound libraries: a new approach to decision making in molecular discovery research. Journal of chemical information and computer sciences 38, 1010-1023 (1998).
Durrant, J. D. & McCammon, J. A. AutoClickChem: click chemistry in silico. PLoS Comput Biol 8, e1002397 (2012).
Follit, C. A., Brewer, F. K., Wise, J. G., & Vogel, P. D. (2015). In silico identified targeted inhibitors of P-glycoprotein overcome multidrug resistance in human cancer cells in culture. Pharmacol Res Perspect, 3(5), e00170. doi:10.1002/prp2.170
Humphrey, W., Dalke, A. & Schulten, K. VMD: visual molecular dynamics. J Mol Graph 14, 33-38 (1996).
Ihlenfeldt, W. D., Voigt, J. H., Bienfait, B., Oellien, F. & Nicklaus, M. C. Enhanced CACTVS browser of the Open NCI Database. J Chem Inf Comput Sci 42, 46-57 (2002).
Ihlenfeldt, W. D., Takahashi, Y., Abe, H. & Sasaki, S.-i. Computation and management of chemical properties in CACTVS: An extensible networked approach toward modularity and compatibility. Journal of chemical information and computer sciences 34, 109-116 (1994).
Krier, M. et al. Design of small-sized libraries by combinatorial assembly of linkers and functional groups to a given scaffold: application to the structure-based optimization of a phosphodiesterase 4 inhibitor. Journal of medicinal chemistry 48, 3816-3822 (2005).
Melnikov, A. A., Palyulin, V. A. & Zefirov, N. S. Generation of molecular graphs for QSAR studies: an approach based on supergraphs. Journal of chemical information and modeling 47, 2077-2088 (2007).
Morris, G. M., R. Huey, W. Lindstrom, M. F. Sanner, R. K. Belew, D. S. Goodsell and A. J. Olson (2009). “AutoDock4 and AutoDockTools4: Automated docking with selective receptor flexibility.” J Comput Chem 30(16): 2785-2791.
Nanayakkara, A. K., Follit, C. A., Chen, G., Williams, N. S., Vogel, P. D., & Wise, J. G. (2018). Targeted inhibitors of P-glycoprotein increase chemotherapeutic-induced mortality of multidrug resistant tumor cells. Sci Rep, 8(1), 967.
O'Boyle, N. M. et al. Open Babel: An open chemical toolbox. Journal of cheminformatics 3, 33 (2011).
Ousterhout, J. K., and Jones, K. (2010) Tcl and the Tk toolkit, 2nd ed., Addison Wesley, Upper Saddle River, N.J.
Pearlman, R. S. & Smith, K. Novel software tools for chemical diversity. Perspectives in Drug Discovery and Design 9, 339-353 (1998).
Srinivasan, R., Uttamchandani, M. & Yao, S. Q. Rapid assembly and in situ screening of bidentate inhibitors of protein tyrosine phosphatases. Organic letters 8, 713-716 (2006).
Trott, O. and A. J. Olson (2010). “AutoDock Vina: improving the speed and accuracy of docking with a new scoring function, efficient optimization, and multithreading.” J Comput Chem 31(2): 455-461.
Wang, Z., H. Sun, X. Yao, D. Li, L. Xu, Y. Li, S. Tian and T. Hou (2016). “Comprehensive evaluation of ten docking programs on a diverse set of protein-ligand complexes: the prediction accuracy of sampling power and scoring power.” Physical Chemistry Chemical Physics 18(18):12964-12975.

### EXAMPLE REFERENCES

[1] Ferreira, R. J., dos Santos, D. J., and Ferreira, M. J. (2015) P-glycoprotein and membrane roles in multidrug resistance, Future Med Chem 7, 929-946.
[2] Sharom, F. J. (2008) ABC multidrug transporters: structure, function and role in chemoresistance, Pharmacogenomics 9, 105-127.
[3] Jaramillo, A. C., Al Saig, F., Cloos, J., Jansen, G., and Peters, G. J. (2018) How to overcome ATP-binding cassette drug efflux transporter-mediated drug resistance?, Cancer Drug Resistance 1, 6-29.
[4] Mao, Q., and Unadkat, J. D. (2015) Role of the breast cancer resistance protein (BCRP/ABCG2) in drug transport—an update, The AAPS journal 17, 65-82.
[5] Zhao, J. (2016) Cancer stem cells and chemoresistance: The smartest survives the raid, Pharmacol Ther 160, 145-158.
[6] Wijdeven, R. H., Pang, B., Assaraf, Y. G., and Neefjes, J. (2016) Old drugs, novel ways out: Drug resistance toward cytotoxic chemotherapeutics, Drug Resist Updat 28, 65-81.
[7] Fruci, D., Cho, W. C., Nobili, V., Locatelli, F., and Alisi, A. (2016) Drug Transporters and Multiple Drug Resistance in Pediatric Solid Tumors, Current drug metabolism 17, 308-316.
[8] Tamaki, A., Ierano, C., Szakacs, G., Robey, R. W., and Bates, S. E. (2011) The controversial role of ABC transporters in clinical oncology, Essays in biochemistry 50, 209-232.
[9] Palmeira, A., Sousa, E., Vasconcelos, M. H., and Pinto, M. M. (2012) Three decades of P-gp inhibitors: skimming through several generations and scaffolds, Current medicinal chemistry 19, 1946-2025.
[10] Binkhathlan, Z., and Lavasanifar, A. (2013) P-glycoprotein inhibition as a therapeutic approach for overcoming multidrug resistance in cancer: current status and future perspectives, Current cancer drug targets 13, 326-346.
[11] Kathawala, R. J., Gupta, P., Ashby, C. R., and Chen, Z.-S. (2015) The modulation of ABC transporter-mediated multidrug resistance in cancer: A review of the past decade, Drug Resistance Updates 18, 1-17.
[12] Gottesman, M. M., and Pastan, I. H. (2015) The Role of Multidrug Resistance Efflux Pumps in Cancer: Revisiting a JNCI Publication Exploring Expression of the MDR1 (P-glycoprotein) Gene, J Natl Cancer Inst 107.
[13] Marin, J. J., Briz, O., Rodriguez-Macias, G., Diez-Martin, J. L., and Macias, R. I. (2016) Role of drug transport and metabolism in the chemoresistance of acute myeloid leukemia, Blood Rev 30, 55-64.
[14] List, A. F., Kopecky, K. J., Willman, C. L., Head, D. R., Persons, D. L., Slovak, M. L., Dorr, R., Karanes, C., Hynes, H. E., Doroshow, J. H., Shurafa, M., and Appelbaum, F. R. (2001) Benefit of cyclosporine modulation of drug resistance in patients with poor-risk acute myeloid leukemia: a Southwest Oncology Group study, Blood 98, 3212-3220.
[15] Li, J., Jaimes, K. F., and Aller, S. G. (2014) Refined structures of mouse P-glycoprotein, Protein Sci 23, 34-46.
[16] Kim, Y., and Chen, J. (2018) Molecular structure of human P-glycoprotein in the ATP-bound, outward-facing conformation, Science, eaar7389.
[17] Pan, L., and Aller, S. G. (2015) Equilibrated atomic models of outward-facing P-glycoprotein and effect of ATP binding on structural dynamics, Scientific reports 5, 7880.
[18] Jin, M. S., Oldham, M. L., Zhang, Q., and Chen, J. (2012) Crystal structure of the multidrug transporter P-glycoprotein from Caenorhabditis elegans, Nature 490, 566-569.
[19] Taylor, N. M. I., Manolaridis, I., Jackson, S. M., Kowal, J., Stahlberg, H., and Locher, K. P. (2017) Structure of the human multidrug transporter ABCG2, Nature 546, 504-509.
[20] Johnson, Z. L., and Chen, J. (2017) Structural Basis of Substrate Recognition by the Multidrug Resistance Protein MRP1, Cell 168, 1075-1085 e1079.
[21] Wise, J. G. (2012) Catalytic transitions in the human MDR1 P-glycoprotein drug binding sites, Biochemistry 51, 5125-5141.
[22] McCormick, J. W., Vogel, P. D., and Wise, J. G. (2015) Multiple Drug Transport Pathways through Human P-Glycoprotein, Biochemistry 54, 4374-4390.
[23] Frank, G. A., Shukla, S., Rao, P., Borgnia, M. J., Bartesaghi, A., Merk, A., Mobin, A., Esser, L., Earl, L. A., and Gottesman, M. M. (2016) Cryo-EM analysis of the conformational landscape of human P-glycoprotein (ABCB1) during its catalytic cycle, Molecular pharmacology 90, 35-41.
[24] Khunweeraphong, N., Stockner, T., and Kuchler, K. (2017) The structure of the human ABC transporter ABCG2 reveals a novel mechanism for drug extrusion, Scientfic reports 7, 13767.
[25] Zoghbi, M. E., Mok, L., Swartz, D. J., Singh, A., Fendley, G. A., Urbatsch, I. L., and Altenberg, G. A. (2017) Substrate-induced conformational changes in the nucleotide-binding domains of lipid bilayer-associated P-glycoprotein during ATP hydrolysis, J Biol Chem 292, 20412-20424.
[26] Brewer, F. K., Follit, C. A., Vogel, P. D., and Wise, J. G. (2014) In silico Screening for Inhibitors of P-Glycoprotein that Target the Nucleotide Binding Domains, Molecular pharmacology 86, 716-726.
[27] Follit, C. A., Brewer, F. K., Wise, J. G., and Vogel, P. D. (2015) In silico identified targeted inhibitors of P-glycoprotein overcome multidrug resistance in human cancer cells in culture, Pharmacol Res Perspect 3, e00170.
[28] Nanayakkara, A. K., Follit, C. A., Chen, G., Williams, N. S., Vogel, P. D., and Wise, J. G. (2018) Targeted inhibitors of P-glycoprotein increase chemotherapeutic-induced mortality of multidrug resistant tumor cells, Scientific Reports 8, 967.
[29] Cramer, R. D., Patterson, D. E., Clark, R. D., Soltanshahi, F., and Lawless, M. S. (1998) Virtual compound libraries: a new approach to decision making in molecular discovery research, Journal of chemical information and computer sciences 38, 1010-1023.
[30] Krier, M., de Araújo-Júnior, J. X., Schmitt, M., Duranton, J., Justiano-Basaran, H., Lugnier, C., Bourguignon, J.-J., and Rognan, D. (2005) Design of small-sized libraries by combinatorial assembly of linkers and functional groups to a given scaffold: application to the structure-based optimization of a phosphodiesterase 4 inhibitor, Journal of medicinal chemistry 48, 3816-3822.
[31] Srinivasan, R., Uttamchandani, M., and Yao, S. Q. (2006) Rapid assembly and in situ screening of bidentate inhibitors of protein tyrosine phosphatases, Organic letters 8, 713-716.
[32] Durrant, J. D., and McCammon, J. A. (2012) AutoClickChem: click chemistry in silico, PLoS computational biology 8, e1002397.
[33] Takeda, M., Mizokami, A., Mamiya, K., Li, Y. Q., Zhang, J., Keller, E. T., and Namiki, M. (2007) The establishment of two paclitaxel-resistant prostate cancer cell lines and the mechanisms of paclitaxel resistance with two cell lines, Prostate 67, 955-967.
[34] Irwin, J. J., Sterling, T., Mysinger, M. M., Bolstad, E. S., and Coleman, R. G. (2012) ZINC: A Free Tool to Discover Chemistry for Biology, Journal of chemical information and modeling 52, 1757-1768.
[35] Moriarty, N. W., Grosse-Kunstleve, R. W., and Adams, P. D. (2009) electronic Ligand Builder and Optimization Workbench (eLBOW): a tool for ligand coordinate and restraint generation, Acta crystallographica. Section D, Biological crystallography 65, 1074-1080.
[36] Yang, Z. H., Son, K. I., Li, S. Q., Zhou, B. N., and Xu, J. X. (2014) Specific 1,2-Hydride Shift in the Boron Trifluoride Catalyzed Reactions of Aromatic Aldehydes with Diazoacetonitrile: Simple Synthesis of beta-Ketonitriles, Eur J Org Chem, 6380-6384.
[37] Su, W. N., Lin, T. P., Cheng, K. M., Sung, K. C., Lin, S. K., and Wong, F. F. (2010) An Efficient One-Pot Synthesis of N-(1,3-Diphenyl-H-Pyrazol-5-yl)amides, J Heterocyclic Chem 47, 831-837.
[38] Hilton, S. T., Motherwell, W. B., Potier, P., Pradet, C., and Selwood, D. L. (2005) Observations on the reactivity of thiyl radicals derived from 3,6-epidithiodiketopiperazine-2,5-diones and related congeners, Bioorganic & Medicinal Chemistry Letters 15, 2239-2242.
[39] Robertson, D. W., Krushinski, J. H., Beedle, E. E., Leander, J. D., Wong, D. T., and Rathbun, R. C. (1986) Structure-Activity-Relationships of (Arylalkyl)Imidazole Anticonvulsants—Comparison of the (Fluorenylalkyl)Imidazoles with Nafimidone and Denzimol, Journal of Medicinal Chemistry 29, 1577-1586.
[40] van Meerloo, J., Kaspers, G. J., and Cloos, J. (2011) Cell sensitivity assays: the MTT assay, Methods Mol Biol 731, 237-245.
[41] Berridge, M. V., and Tan, A. S. (1993) Characterization of the cellular reduction of 3-(4,5-dimethylthiazol-2-yl)-2,5-diphenyltetrazolium bromide (MTT): subcellular localization, substrate dependence, and involvement of mitochondrial electron transport in MTT reduction, Arch Biochem Biophys 303, 474-482.
[42] Kupcsik, L. (2011) Estimation of cell number based on metabolic activity: the MTT reduction assay, Methods Mol Biol 740, 13-19.
[43] Mannhold, R., Poda, G. I., Ostermann, C., and Tetko, I. V. (2009) Calculation of Molecular Lipophilicity: State-of-the-Art and Comparison of Log P Methods on More Than 96,000 Compounds, Journal of pharmaceutical sciences 98, 861-893.
[44] Daina, A., Michielin, O., and Zoete, V. (2017) SwissADME: a free web tool to evaluate pharmacokinetics, drug-likeness and medicinal chemistry friendliness of small molecules, Scientfic reports 7, 42717.
[45] Lerner-Marmarosh, N., Gimi, K., Urbatsch, I. L., Gros, P., and Senior, A. E. (1999) Large scale purification of detergent-soluble P-glycoprotein from Pichia pastoris cells and characterization of nucleotide binding properties of wild-type, Walker A, and Walker B mutant proteins, J Biol Chem 274, 34711-34718.
[46] Tombline, G., Urbatsch, I. L., Virk, N., Muharemagic, A., White, L. B., and Senior, A. E. (2006) Expression, purification, and characterization of cysteine-free mouse P-glycoprotein, Arch Biochem Biophys 445, 124-128.
[47] Martin, C., Berridge, G., Mistry, P., Higgins, C., Charlton, P., and Callaghan, R. (1999) The molecular interaction of the high affinity reversal agent XR9576 with P-glycoprotein, Br J Pharmacol 128, 403-411.
[48] Streckenbach, B., Schwarz, D., and Repke, K. R. (1980) Analysis of phosphoryl transfer mechanism and catalytic centre geometries of transport ATPase by means of spin-labelled ATP, Biochim Biophys Acta 601, 34-46.
[49] Delannoy, S., Urbatsch, I. L., Tombline, G., Senior, A. E., and Vogel, P. D. (2005) Nucleotide binding to the multidrug resistance P-glycoprotein as studied by ESR spectroscopy, Biochemistry 44, 14010-14019.
[50] Vogel, P. D., Nett, J. H., Sauer, H. E., Schmadel, K., Cross, R. L., and Trommer, W. E. (1992) Nucleotide binding sites on mitochondrial F1-ATPase. Electron spin resonance spectroscopy and photolabeling by azido-spin-labeled adenine nucleotides support an adenylate kinase-like orientation, J Biol Chem 267, 11982-11986.
[51] Breul, S. D., Bradley, K. H., Hance, A. J., Schafer, M. P., Berg, R. A., and Crystal, R. G. (1980) Control of collagen production by human diploid lung fibroblasts, J Biol Chem 255, 5250-5260.
[52] Stone, K. R., Mickey, D. D., Wunderli, H., Mickey, G. H., and Paulson, D. F. (1978) Isolation of a human prostate carcinoma cell line (DU 145), Int J Cancer 21, 274-281.
[53] Morgan, S., Grootendorst, P., Lexchin, J., Cunningham, C., and Greyson, D. (2011) The cost of drug development: A systematic review, Health Policy 100, 4-17.
[54] Merk, D., Friedrich, L., Grisoni, F., and Schneider, G. (2018) De novo design of bioactive small molecules by artificial intelligence, Molecular informatics 37, 1700153.
[55] Chen, H., Engkvist, O., Wang, Y., Olivecrona, M., and Blaschke, T. (2018) The rise of deep learning in drug discovery, Drug discovery today.
[56] Gomes, J., Ramsundar, B., Feinberg, E. N., and Pande, V. S. (2017) Atomic convolutional networks for predicting protein-ligand binding affinity, arXiv preprint arXiv:1703.10603.
[57] Goh, G. B., Hodas, N. O., and Vishnu, A. (2017) Deep learning for computational chemistry, Journal of computational chemistry 38, 1291-1307.
[58] Altae-Tran, H., Ramsundar, B., Pappu, A. S., and Pande, V. (2017) Low data drug discovery with one-shot learning, ACS central science 3, 283-293.
[59] Subramanian, G., Ramsundar, B., Pande, V., and Denny, R. A. (2016) Computational modeling of p-secretase 1 (BACE-1) inhibitors using ligand based approaches, Journal of chemical information and modeling 56, 1936-1949.
[60] Trott, O., and Olson, A. J. (2010) AutoDock Vina: improving the speed and accuracy of docking with a new scoring function, efficient optimization, and multithreading, J Comput Chem 31, 455-461.
[61] Goodsell, D. S., Morris, G. M., and Olson, A. J. (1996) Automated docking of flexible ligands: applications of AutoDock, J Mol Recognit 9, 1-5.
[62] Morris, G. M., Goodsell, D. S., Huey, R., and Olson, A. J. (1996) Distributed automated docking of flexible ligands to proteins: parallel applications of AutoDock 2.4, J Comput Aided Mol Des 10, 293-304.
[63] Osterberg, F., Morris, G. M., Sanner, M. F., Olson, A. J., and Goodsell, D. S. (2002) Automated docking to multiple target structures: incorporation of protein mobility and structural water heterogeneity in AutoDock, Proteins 46, 34-40.
[64] Dawson, R. J. P., and Locher, K. P. (2006) Structure of a bacterial multidrug ABC transporter, Nature 443, 180-185.
[65] Humphrey, W., Dalke, A., and Schulten, K. (1996) VMD: visual molecular dynamics, J Mol Graph 14, 33-38.
[66] Varshney, A., Brooks, F. P., and Wright, W. V. (1994) Computing smooth molecular surfaces, IEEE Computer Graphics and Applications 14, 19-25.
[67] Dolinsky, T. J., Nielsen, J. E., McCammon, J. A., and Baker, N. A. (2004) PDB2PQR: an automated pipeline for the setup of Poisson-Boltzmann electrostatics calculations, Nucleic acids research 32, W665-W667.
[68] Dolinsky, T. J., Czodrowski, P., Li, H., Nielsen, J. E., Jensen, J. H., Klebe, G., and Baker, N. A. (2007) PDB2PQR: expanding and upgrading automated preparation of biomolecular structures for molecular simulations, Nucleic acids research 35, W522-W525.
[69] Baker, N. A., Sept, D., Joseph, S., Holst, M. J., and McCammon, J. A. (2001) Electrostatics of nanosystems: application to microtubules and the ribosome, Proceedings of the National Academy of Sciences 98, 10037-10041.
[70] Jurrus, E., Engel, D., Star, K., Monson, K., Brandi, J., Felberg, L. E., Brookes, D. H., Wilson, L., Chen, J., and Liles, K. (2018) Improvements to the APBS biomolecular solvation software suite, Protein Science 27, 112-128.
[71] Wu, C.-Y., Feng, Y., Cardenas, E. R., Williams, N., Floreancig, P. E., De Brabander, J. K., and Roth, M. G. (2012) Studies toward the unique pederin family member psymberin: structure-activity relationships, biochemical studies, and genetics identify the mode-of-action of psymberin, Journal of the American Chemical Society 134, 18998-19003.
[72] Vogel, G., and Steinhart, R. (1976) ATPase of Escherichia coli: purification, dissociation, and reconstitution of the active complex from the isolated subunits, Biochemistry 15, 208-216.
[73] Denisov, I. G., Grinkova, Y. V., Lazarides, A. A., and Sligar, S. G. (2004) Directed self-assembly of monodisperse phospholipid bilayer Nanodiscs with controlled size, J Am Chem Soc 126, 3477-3487.
[74] Ritchie, T. K., Grinkova, Y. V., Bayburt, T. H., Denisov, I. G., Zolnerciks, J. K., Atkins, W. M., and Sligar, S. G. (2009) Chapter 11—Reconstitution of membrane proteins in phospholipid bilayer nanodiscs, Methods Enzymol 464, 211-231.

### SUPPORTING INFORMATION REFERENCES

[1] Humphrey, W., Dalke, A., and Schulten, K. (1996) VMD: visual molecular dynamics, J Mol Graph 14, 33-38.
[2] O'Boyle, N. M., Banck, M., James, C. A., Morley, C., Vandermeersch, T., and Hutchison, G. R. (2011) Open Babel: An open chemical toolbox, Journal of cheminformatics 3, 33.
[3] Ihlenfeldt, W. D., Takahashi, Y., Abe, H., and Sasaki, S.-i. (1994) Computation and management of chemical properties in CACTVS: An extensible networked approach toward modularity and compatibility, Journal of chemical information and computer sciences 34, 109-116.
[4] Ihlenfeldt, W.-D., Voigt, J. H., Bienfait, B., Oellien, F., and Nicklaus, M. C. (2002) Enhanced CACTVS browser of the Open NCI Database, J Chem Inf Comput Sci 42, 46-57.
[5] Ousterhout, J. K., and Jones, K. (2010) Tcl and the Tk toolkit, 2nd ed., Addison Wesley, Upper Saddle River, N.J.
[6] Moriarty, N. W., Grosse-Kunstleve, R. W., and Adams, P. D. (2009) electronic Ligand Builder and Optimization Workbench (eLBOW): a tool for ligand coordinate and restraint generation, Acta crystallographica. Section D, Biological crystallography 65, 1074-1080.
[7] Wang, J., Wang, W., Kollman, P. A., and Case, D. A. (2001) Antechamber: an accessory software package for molecular mechanical calculations, J. Am. Chem. Soc 222, U403.
[8] Schmidt, M. W., Baldridge, K. K., Boatz, J. A., Elbert, S. T., Gordon, M. S., Jensen, J. H., Koseki, S., Matsunaga, N., Nguyen, K. A., Su, S. J., Windus, T. L., Dupuis, M., and Montgomery, J. A. (1993) General Atomic and Molecular Electronic-Structure System, Journal of Computational Chemistry 14, 1347-1363.
[9] Nanayakkara, A. K., Follit, C. A., Chen, G., Williams, N. S., Vogel, P. D., and Wise, J. G. (2018) Targeted inhibitors of P-glycoprotein increase chemotherapeutic-induced mortality of multidrug resistant tumor cells, Scientific reports 8, 967.
[10] Takeda, M., Mizokami, A., Mamiya, K., Li, Y. Q., Zhang, J., Keller, E. T., and Namiki, M. (2007) The establishment of two paclitaxel-resistant prostate cancer cell lines and the mechanisms of paclitaxel resistance with two cell lines, Prostate 67, 955-967.
[11] Yang, Z. H., Son, K. I., Li, S. Q., Zhou, B. N., and Xu, J. X. (2014) Specific 1,2-Hydride Shift in the Boron Trifluoride Catalyzed Reactions of Aromatic Aldehydes with Diazoacetonitrile: Simple Synthesis of beta-Ketonitriles, Eur J Org Chem, 6380-6384.
[12] Su, W. N., Lin, T. P., Cheng, K. M., Sung, K. C., Lin, S. K., and Wong, F. F. (2010) An Efficient One-Pot Synthesis of N-(1,3-Diphenyl-H-Pyrazol-5-yl)amides, J Heterocyclic Chem 47, 831-837.
[13] Saravanan, G., Selvaraju, R., and Nagarajan, S. (2012) Synthesis of Novel 2-Iminothiazolidin-4-Ones, Synthetic Commun 42, 3361-3367.
[14] Marcu, A., Schurigt, U., Muller, K., Moll, H., Krauth-Siegel, R. L., and Prinz, H. (2016) Inhibitory effect of phenothiazine- and phenoxazine-derived chloroacetamides on Leishmania major growth and Trypanosoma brucei trypanothione reductase, European Journal of Medicinal Chemistry 108, 436-443.
[15] Zhao, P.-L., Chen, P., Li, Q., Hu, M.-J., Diao, P.-C., Pan, E.-S., and You, W.-W. (2016) Design, synthesis and biological evaluation of novel 3-alkylsulfanyl-4-amino-1,2,4-triazole derivatives, Bioorganic & Medicinal Chemistry Letters 26, 3679-3683.
[16] Heil, M. L., Cosford, N. D., Ardecky, R., and Zou, J. (2017) Quinazolinone analogs and use of quinazolinone analogs for treating or preventing certain viral infections, (Office, U. S. P. a. T., Ed.), Sanford-Burnham Prebys Medical Discovery Institute, Southern Research Institute, US.
[17] Toscan, C. E., Rahimi, M., Bhadbhade, M., Pickford, R., McAlpine, S. R., and Lock, R. B. (2015) Thioimidazoline based compounds reverse glucocorticoid resistance in human acute lymphoblastic leukemia xenografts, Organic & biomolecular chemistry 13, 6299-6312.
[18] Beisel, T., and Manolikakes, G. (2013) A Lewis Acid Palladium(II)-Catalyzed Three-Component Synthesis of alpha-Substituted Amides, Organic Letters 15, 6046-6049.
[19] Li, Y.-l., Qi, X.-y., Jiang, H., Deng, X.-d., Dong, Y.-p., Ding, T.-b., Zhou, L., Men, P., Chu, Y., Wang, R.-x., Jiang, X.-c., and Ye, D.-y. (2015) Discovery, synthesis and biological evaluation of 2-(4-(N-phenethylsulfamoyl)phenoxy)acetamides (SAPAs) as novel sphingomyelin synthase 1 inhibitors, Bioorganic & Medicinal Chemistry 23, 6173-6184.
[20] Robertson, D. W., Krushinski, J. H., Beedle, E. E., Leander, J. D., Wong, D. T., and Rathbun, R. C. (1986) Structure-Activity-Relationships of (Arylalkyl)Imidazole Anticonvulsants—Comparison of the (Fluorenylalkyl)Imidazoles with Nafimidone and Denzimol, Journal of Medicinal Chemistry 29, 1577-1586.
[21] Gianella-Borradori, M., Christou, I., Bataille, C. J., Cross, R. L., Wynne, G. M., Greaves, D. R., Russell, A. J. (2016) Ligand-based virtual screening identifies a family of selective cannabinoid receptor 2 agonists. Biorganic & Medicinal Chemistry 23, 241-263.
[22] Tao, C., Wang, Q., Nallan, L., Polat, T., Koroniak, L. and Desai, N., California Capital Equity LLC, 2012. Triazine Derivatives and their Therapeutical Applications. U.S. patent application Ser. No. 13/376,964.

What is claimed is:

1. A method for identifying one or more potentially useful molecular combinations comprising:
applying a selection procedure to a compound of interest to identify a first set of one or more candidate molecules, the selection procedure comprising:
providing a chemical synthesis scheme for a compound of interest, a virtual scaffold molecule of the compound of interest, and a virtual reactant fragment to react with the virtual scaffold molecule according to the chemical synthesis scheme;
preparing the virtual reactant fragment and the virtual scaffold molecule for analyzing combinations of the virtual reactant fragment and the virtual scaffold molecule;
designating a remaining scaffold subset and a remaining fragment subset if a product molecule can be formed from the virtual scaffold molecule and the virtual reactant fragment;
rotating the remaining fragment subset about an axis connecting the remaining scaffold subset and the remaining fragment subset through 360 degrees in increments of less than or equal to 5 degrees; and
identifying potentially useful combinations of the virtual reactant fragment and the virtual scaffold molecule, by:
recording as a potential product increment each increment at which a steric collision is not detected; and
recording a separation distance between the remaining fragment subset and the remaining scaffold subset at each increment and identifying a set of product increments for which the separation distances are less than or equal to a predetermined criterion distance to identify the one or more potentially useful molecular combinations;
identifying a set of combinatorial fragments from the first set of one or more candidates; and
applying the selection procedure to the set of combinatorial fragments to identify a second set of one or more candidate molecules that are the one or more potentially useful molecular combinations.

2. The method of claim 1, wherein:
the preparing the virtual reactant fragment and the virtual scaffold molecule comprises providing a three-dimensional coordinate system for the virtual reactive fragment.

3. The method of claim 1, wherein:
the preparing the virtual reactant fragment and the virtual scaffold molecule comprises identifying a fragment alignment atom and a fragment root atom in the virtual reactant fragment; and
the preparing the virtual reactant fragment and the virtual scaffold molecule comprises:
identifying a scaffold alignment atom and a scaffold root atom in the virtual scaffold molecule; and
providing a three-dimensional coordinate system for the virtual scaffold molecule and aligning the scaffold root atom with an origin and the scaffold alignment atom with an x-axis.

4. The method of claim 3, wherein
the preparing the virtual reactant fragment and the virtual scaffold molecule comprises:
aligning the fragment alignment atom with the scaffold root atom; and
aligning the fragment root atom with the scaffold alignment atom.

5. The method of claim 4, wherein the axis connecting the remaining scaffold subset and the remaining fragment subset is defined by the scaffold root atom and the virtual root atom.

6. The method of claim 1, wherein:
the identifying potentially useful combinations further comprises creating a product file for a configuration of the remaining fragment subset and the remaining scaffold subset at each increment of the set of product increments.

7. A non-transitory computer-readable medium encoded with a computer program for execution by a processor for identifying one or more potentially useful molecular combinations, the computer program comprising instructions for:
applying a selection procedure to a compound of interest to identify a first set of one or more candidate molecules, the selection procedure comprising:
receiving a chemical synthesis scheme for a compound of interest, a virtual scaffold molecule of the compound of interest, and a virtual reactant fragment to react with the virtual scaffold molecule according to the chemical synthesis scheme;
receiving input to prepare the virtual reactant fragment and the virtual scaffold molecule for analyzing combinations of the virtual reactant fragment and the virtual scaffold molecule;
designating a remaining scaffold subset and a remaining fragment subset if a product molecule can be formed from the virtual scaffold molecule and the virtual reactant fragment;
rotating the remaining fragment subset about an axis connecting the remaining scaffold subset and the remaining fragment subset through 360 degrees in increments of less than or equal to 5 degrees;
identifying potentially useful combinations of the virtual reactant fragment and the virtual scaffold molecule by:
recording as a potential product increment each increment at which a steric collision is not detected; and
recording a separation distance between the remaining fragment subset and the remaining scaffold subset at each increment and identifying a set of product increments for which the separation distances are less than or equal to a predetermined criterion distance, to identify the first set of one or more candidate molecules; and
identifying a set of combinatorial fragments from the first set of one or more candidates; and
applying the selection procedure to the set of combinatorial fragments to identify a second set of one or more candidate molecules that are the one or more potentially useful molecular combinations.

8. The medium of claim 7, wherein:
the preparing the virtual reactant fragment and the virtual scaffold molecule comprises providing a three-dimensional coordinate system for the virtual reactive fragment.

9. The medium of claim 7, wherein:
the preparing the virtual reactant fragment and the virtual scaffold molecule comprises identifying a fragment alignment atom and a fragment root atom in the virtual reactant fragment; and
the preparing the virtual reactant fragment and the virtual scaffold molecule comprises:
identifying a scaffold alignment atom and a scaffold root atom in the virtual scaffold molecule; and
providing a three-dimensional coordinate system for the virtual scaffold molecule and aligning the scaffold root atom with an origin and the scaffold alignment atom with an x-axis.

10. The medium of claim 9, wherein
the preparing the virtual reactant fragment and the virtual scaffold molecule comprises:
aligning the fragment alignment atom with the scaffold root atom; and
aligning the fragment root atom with the scaffold alignment atom.

11. The medium of claim 10, wherein the axis connecting the remaining scaffold subset and the remaining fragment subset is defined by the scaffold root atom and the virtual root atom.

12. The medium of claim 7, wherein:
the identifying potentially useful combinations further comprises creating a product file for a configuration of the remaining fragment subset and the remaining scaffold subset at each increment of the set of product increments.

13. An apparatus for identifying one or more potentially useful molecular combinations comprising:
a processor;
a memory communicably coupled to the processor;
an output device communicably coupled to the processor; and
a non-transitory computer-readable medium encoded with a computer program for execution by the processor that causes the processor to:
apply a selection procedure to a compound of interest to identify a first set of one or more candidate molecules, the selection procedure comprising:
receiving a chemical synthesis scheme for a compound of interest, a virtual scaffold molecule of the compound of interest, and a virtual reactant fragment to react with the virtual scaffold molecule according to the chemical synthesis scheme;
receiving input to prepare the virtual reactant fragment and the virtual scaffold molecule for analyzing combinations of the virtual reactant fragment and the virtual scaffold molecule;
designating a remaining scaffold subset and a remaining fragment subset if a product molecule can be formed from the virtual scaffold molecule and the virtual reactant fragment;
rotating the remaining fragment subset about an axis connecting the remaining scaffold subset and the remaining fragment subset through 360 degrees in increments of less than or equal to 5 degrees; and
identifying potentially useful combinations of the virtual reactant fragment and the virtual scaffold molecule, by:
recording as a potential product increment each increment at which a steric collision is not detected; and
recording a separation distance between the remaining fragment subset and the remaining scaffold subset at each increment and identifying a set of product increments for which the separation distances are less than or equal to a predetermined criterion distance, to identify the first set of one or more candidate molecules; and
identify a set of combinatorial fragments from the first set of one or more candidates; and
apply the selection procedure to the set of combinatorial fragments to identify a second set of one or more candidate molecules that are the one or more potentially useful molecular combinations.

14. The apparatus of claim 13, wherein:
the preparing the virtual reactant fragment and the virtual scaffold molecule comprises providing a three-dimensional coordinate system for the virtual reactive fragment.

15. The apparatus of claim 13, wherein:
the preparing the virtual reactant fragment and the virtual scaffold molecule comprises identifying a fragment alignment atom and a fragment root atom in the virtual reactant fragment; and
the preparing the virtual reactant fragment and the virtual scaffold molecule comprises:
identifying a scaffold alignment atom and a scaffold root atom in the virtual scaffold molecule; and
providing a three-dimensional coordinate system for the virtual scaffold molecule and aligning the scaffold root atom with an origin and the scaffold alignment atom with an x-axis.

16. The apparatus of claim 15, wherein
the preparing the virtual reactant fragment and the virtual scaffold molecule comprises:
aligning the fragment alignment atom with the scaffold root atom; and
aligning the fragment root atom with the scaffold alignment atom.

17. The method of claim 16, wherein the axis connecting the remaining scaffold subset and the remaining fragment subset is defined by the scaffold root atom and the virtual root atom.

18. The apparatus of claim 13, wherein:
the identifying potentially useful combinations further comprises creating a product file for a configuration of the remaining fragment subset and the remaining scaffold subset at each increment of the set of product increments.
